SEQUENCE LISTING (15 sequences)

SEQ ID NO: 1 - DNA, 33 bp, Artificial Sequence
  Description of Artificial Sequence Primer
ggaaggaaaaaagcggccgcaacannnnnnnnn

SEQ ID NO: 2 - DNA, 23 bp, Artificial Sequence
  Description of Artificial Sequence Primer
aatccgatgcccacgttgcagta

SEQ ID NO: 3 - DNA, 26 bp, Artificial Sequence
  Description of Artificial Sequence Primer
aaaatcttagaccgacgactgtgttt

SEQ ID NO: 4 - DNA, 22 bp, Artificial Sequence
  Description of Artificial Sequence Primer
gagtctccgcagccttttgagg

SEQ ID NO: 5 - DNA, 357 bp, Mus musculus
  CDS (6)..(356)
cagccatggttaccttcagccacgtctccagtctgagtcactggttcctcttgctgctgctgctgaatctgttcttgccggtaatatttgctatgcctgaatcatactccttcaactgtcccgatggtgaataccagtctaatgatgtctgttgcaagacctgtccctcaggtacatttgtcaaggcgccctgcaaaatcccccatactcaaggacaatgtgagaagtgtcacccaggaacattcacagggaaagataatggcctgcatgattgtgaactttgctccacctgtgataaagaccagaatatggtggctgactgttctgccaccagtgaccggaaatgcgagtgccaaa

SEQ ID NO: 6 - protein, 117 aa, Mus musculus
MVTFSHVSSLSHWFLLLLLLNLFLPVIFAMPESYSFNCPDGEYQSNDVCCKTCPSGTFVKAPCKIPHTQGQCEKCHPGTFTGKDNGLHDCELCSTCDKDQNMVADCSATSDRKCECQ

SEQ ID NO: 7 - DNA, 967 bp, Mus musculus
  CDS (4)..(531): Mu-ymkz5
gccatggttaccttcagccacgtctccagtctgagtcactggttcctcttgctgctgctgctgaatctgttcttgccggtaatatttgctatgcctgaatcatactccttcaactgtcccgatggtgaataccagtctaatgatgtctgttgcaagacctgtccctcaggtacatttgtcaaggcgccctgcaaaatcccccatactcaaggacaatgtgagaagtgtcacccaggaacattcacagggaaagataatggcctgcatgattgtgaactttgctccacctgtgataaagaccagaatatggtggctgactgttctgccaccagtgaccggaaatgcgagtgccaaataggtctttactactatgacccaaaatttccggaatcatgccgcccatgtaccaagtgtccccaaggaatccctgtcctccaggaatgcaactccacagctaacactgtgtgcagttcatctgtttcaaatcccagaaactggctgttcctactgatgctaattgtcttctgtatctgaagaagataaaggttctacagatggtgtctgtagcttccttttattgctgtgaagagaaaccatggaggcaactctttcattttattttattttttaatgtcttgaacttgatttgaagaccaggctggactcaaactcacagagatccggactaggcacctctaatataggaaaacattgaattgggactggcttacagtttcagaagttctgtccatgattatcatagtgcgaagcatggaggcacggaggcacacatggtgctggagaagaagctgagagttctgcatcttgatctgcaagcaataaaaggagactgtgtgccacactacacatagcttgaacataggagacctcaaagcctgtccccacagtgacaaacttcctccaacaaggtcatacctcctaataataccatttcttatgaggccct

SEQ ID NO: 8 - protein, 176 aa, Mus musculus
MVTFSHVSSLSHWFLLLLLLNLFLPVIFAMPESYSFNCPDGEYQSNDVCCKTCPSGTFVKAPCKIPHTQGQCEKCHPGTFTGKDNGLHDCELCSTCDKDQNMVADCSATSDRKCECQIGLYYYDPKFPESCRPCTKCPQGIPVLQECNSTANTVCSSSVSNPRNWLFLLMLIVFCI

SEQ ID NO: 9 - protein, 242 aa, Mus musculus
  FASA
MLWIWAVLPLVLAGSQLRVHTQGTNSISESLKLRRRVHETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYRKYRKRKCWKRRQDDPESRTSSRETIPMNASNLSLSKYIPRIAEDMTIQEAKKFAR

SEQ ID NO: 10 - protein, 247 aa, Mus musculus
  TNFR1
MGLPTVPGLLLSLVLLALLMGIHPSGVTGLVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDSGTAVLLPLVILLGLCLLSFIFISLMCRYPRWRPEVYSI

SEQ ID NO: 11 - protein, 84 aa, Mus musculus
  CONS
MVLLLLLLIEDNCPGYQNCCCGTVDCKPGCCGTFTKNYLCCTCDKEVCATDTKCCKFYYDSECVCCGTLPCTNTVCRWLKSTLS

SEQ ID NO: 12 - DNA, 34 bp, Artificial Sequence
  Description of Artificial Sequence Primer
ggtaagcttcaccatggttaccttcagccacgtc

SEQ ID NO: 13 - DNA, 40 bp, Artificial Sequence
  Description of Artificial Sequence Primer
gaattagcggccgcatttgaaacagatgaactgcacacac

SEQ ID NO: 14 - protein, 396 aa, Mus musculus
  ymkz5-Fc fusion protein
MVTFSHVSSLSHWFLLLLLLNLFLPVIFAMPESYSFNCPDGEYQSNDVCCKTCPSGTFVKAPCKIPHTQGQCEKCHPGTFTGKDNGLHDCELCSTCDKDQNMVADCSATSDRKCECQIGLYYYDPKFPESCRPCTKCPQGIPVLQECNSTANTVCSSSVSNAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 11 aa, Artificial Sequence
  Description of Artificial Sequence peptide
YGRKKRRQRRR

(19) United States
(12) Patent Application Publication
(10) Pub. No.: US 2003/0096355 A1
(43) Pub. Date: May 22, 2003

(54) Isolation, identification and characterization of ymkz5, a novel member of the TNF-receptor supergene family

(76) Inventor: Ke Zhang, Thousand Oaks, CA (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/193,616

(22) Filed: Jul. 11, 2002

Related U.S. Application Data
(63) Continuation of application No. 09/611,989, filed on Jul. 7, 2000, now abandoned.
(60) Provisional application No. 60/143,137, filed on Jul. 9, 1999.

(51) Int. Cl. C07K 14/715; C12P 21/02; C12N 5/06; C07H 21/04

(52) U.S. Cl. 435/69.1; 435/320.1; 435/325; 530/350; 536/23.5

(57) ABSTRACT

Novel TNF receptor polypeptides are disclosed, along with polynucleotides encoding the polypeptides and uses thereof.

### RELATED APPLICATIONS

This application claims priority from U.S. provisional patent application Serial No. 60/143,137 filed Jul. 9, 1999.

### FIELD OF THE INVENTION

The invention is in the field of recombinant genetics. In particular, the present invention relates to a novel transmembrane decoy-receptor, ymkz5, belonging to the TNF-receptor supergene family and nucleic acid molecules encoding same. The invention also relates to vectors, host cells, antibodies and recombinant methods for producing both the membrane associated and the soluble forms of the receptor polypeptides. The invention also relates to the use of the recombinant ymkz5 receptor polypeptide to identify putative binding proteins. In addition, methods and reagents are provided for the diagnosis of diseases associated with abnormal expression of ymkz5 or abnormal expression of its putative ligand, and methods and pharmaceutical composition(s) for the treatment, amelioration and/or prevention of diseases associated with expression of abnormal ymkz5 protein or abnormal expression of ymkz5 and/or its ligand. The invention also discloses pharmaceutical compositions for use in the treatment of these diseases.

### BACKGROUND OF THE INVENTION

Technical advances in the identification, cloning, expression and manipulation of nucleic acid molecules have greatly accelerated the discovery of novel therapeutics based upon deciphering the human genome. Rapid nucleic acid sequencing techniques can now generate sequence information at unprecedented rates, and coupled with computational analyses, allow the assembly of overlapping sequences into the entire genome and the identification of polypeptide-encoding, regions. Comparison of a predicted amino acid sequence against a database compilation of known amino acid sequences can allow one to determine the extent of homolology to previously identified sequences and/or structure landmarks. Cloning and expression of a polypeptide-encoding region of a nucleic acid molecule provides a polypeptide product for structural and functional analysis. Manipulation of a nucleic acid molecule(s) and encoded polypeptide(s) to give variants and derivatives thereof may confer advantageous properties on a product for use as a therapeutic.

However, in spite of the significant technical advances in genome research over the past decade the potential for development of novel therapeutics based on the human genome is still largely unrealized. While a number of genes encoding potentially beneficial protein therapeutics or those encoding, polypeptides which may act as “targets” for therapeutic molecules, have been identified using recombinant DNA technology, the structure and function of a vast number of genes in the genome of mammals are yet unknown.

Using the above mentioned recombinant DNA technology, a new member of the tumor necrosis factor (TNF)-receptor supergene family, hereinafter referred to as “ymkz5”, has been identified which may elicit its effects by binding and neutralizing a member of the TNF-family of ligands,

Identification and Characterization of TNF-Family of Ligands and Receptors

Tumor necrosis factor (TNF) was first identified in the serum of mice and rabbits which had been infected with bacillus of Calmette and Guerini(BCG) and which had been injected with endotoxin. TNF activity in the serum of these animals was recognized on the basis of its cytotoxic and anti-tumor activities. This TNF activity. referred to as TNF-α, is produced particularly by activated monocytes and macrophages, and has been implicated in normal growth processes as well as in a variety of diseases.

Following the discovery of TNF-α, independent research led to the identification of another cytokine associated with inflammatory responses lymphotoxin-α (LT-α) which was shown to be produced exclusively by lymphocytes. LT-α was subsequently shown to be 30% homologous with TNF-α, and was renamed TNF-β. It is now clear that TNF-α and TNF-β are members of a (gene family that includes yet another member termed LT-β (Browning et al., Cell 72:847-856 (1993)). The three genes are tightly linked within the MHC complex and show similar organization. Moreover, the biologically active forms of TNF-α and TNF-β are homotrimers and share many of the same biological activities, including competing for the same cell-surface receptors (Agarwal et al , Nature 665-667 (1985)). Two distinct but structurally homologous receptors have been identified, and each has been shown to bind both the ligands and mediate their effects.

However, it has been recognized that TNFs are only representative members of the rapidly expanding supergene family that includes TNF-α, TNF-β/lymphotoxin-α (LT-α), lymphotoxin -β (LT-β), FasL, CD40L, CD30L, CD27L, 4-1BBL, and TNF-related apoptosis-inducing ligand (TRAIL), RANKL, GITRL and TNF-2. The distinctive but overlapping cellular responses induced by members of the TNF family of ligands following their interaction(s) with their cognate cell-surface receptors result in clearly defined developmental and regulatory changes in cells of the lymphoid, hematopoietic, and other lineages. For example, TNF family of ligands are involved in growth regulation and differentiation of cells which are involved in inflamation, immune processes and hematopoiesis (Bayert and Fiers, Tumor Factor and Lymphokines in: Cytokines eds. Anthony Mire-Sluis and Robin Thorpe, Academic Press San Diego Calif. (1998)). The TNF family of ligands activate the immune defenses against parasites, and acts directly and/or indirectly as a mediators in immune reactions, and inflammatory processes. However, administration of TNF and/or other members of the TNF family can also be accompanied by harmful phenomena such as shock and tissue damage (Bayert, and Fiers, supra). The main physiological role of the TNF family of ligands is likely the activation of first-line reaction of an organism to microbial, parasitic, viral, or mechanical stress and cancer. For example, TNF-related apoptosis-inducing ligand (TRAIL) has been demonstrated to induce apoptosis of a number of different types of cancer cells as well as virally infected cells.

Furthermore, a number of observations have also led to the conclusion that TNF family of ligands are also involved in a variety of pathological conditions including cachexia, toxic shock syndrome, inflammatory diseases such as rheumatoid and osteoarthritis, in the lethality resulting from graft-versus-host reaction (GVHR)(Bayert and Fiers, supra), rapid necrosis of tumors, apoptosis, immunostimulation and resistance to parasites and viruses.

Like other cytokines, the TNF family of ligands act via specific cell-surface receptors. The receptors with two exceptions are type 1 member proteins with sequence homology among them almost entirely confined to the extracellular domain. For example, two TNF receptors have been cloned which differ in size and in binding affinity (Bayert and Fiers, supra). Both receptors bind TNF-α and TNF-β. The two receptors consist of extracellular domains which bind TNF and are 28% homologous, transmembrane domains, and intracellular regions an which are totally distinct and do not contain any recognizable structure associated with any particular function. Based on similarities in the extracellular domains, these receptors belong to a receptor gene superfamily that include the low-affinity nerve growth factor (NGF) receptor, the Fas antigen, the human B-lymphocyte activation molecule CD40. CD27, 4-1BB, PV-T2, CD30), TNFR-RP, TRAIL-R, PV-A53R, RANK, GITR, and the OX40 antigen found on activated T-cells (Smith et al., Cell, 76: 959-62 (1994); Baker and Reddy, Oncogene 12: 1-9 (1996)).

In addition to the membrane-associated receptor molecules described above, a number the receptors belonging to the TNF-receptor supergene family exist as soluble binding proteins. Many of the soluble forms of the transmembrane receptors were subsequently identified as containing only the extracellular ligand-binding domain(s) of the receptors. For example, a soluble form of a TNF receptor has been found in urine and serum (See U.S. Pat. No. 5,843,789 and Nophar et al., EMBO J., 9: 3269-78 (1990)), and has been shown to arise from proteolytic cleavage of cell surface TNF-receptors (Porteu et al., J. Biol Chem., 266. 18846-53 (1991)). These soluble forms of receptor molecules have been implicated in the modulation of TNF activity by not only interfering with TNF binding to its receptor, but also by stabilizing the structure and preserving its activity, thus prolonging some of its effects (Aderka et al, Cytokine & Growth Factor Reviews, 7(3):231-240 (1996)).

The activity of TNF family of ligands are tightly regulated at the levels of secretion and receptor expression. Additional regulatory mechanisms are provided by action of specific inhibitory proteins present on cell surface and in biological fluids. While some of these inhibitory proteins have been identified as soluble forms of receptor molecules, the identity of many of these cytokine regulatory proteins are as yet unknown. However, abnormalities in the production of these substances might contribute to the pathophysiology of a variety of diseases including immune and neoplastic diseases. Besides their role in regulating cytokine activity in vivo, these regulatory molecules hold significant potential for therapeutic use as very specific inhibitors anti-cytokine agents, and as indicators in diagnosis and assessment of immune function and growth parameters in a variety of autoimmune and malignant diseases (Fernandez-Botran, F ASEB J., 5:2567-74 (1991)).

Accordingly, it is an object of the invention to identify novel nucleic acid molecules encoding TNF-receptor(s) related molecule(s) that regulate the activity of TNF family of ligands, and to determine their role in a variety of disease processes, and their use as diagnostic and/or therapeutic molecules of diseases.

### SUMMARY OF THE INVENTION

The present invention relates to novel ymkz5-receptor nucleic acid molecules and encoded polypeptides.

The invention provides for an isolated nucleic acid molecule comprising a nucleotide sequence selected from the group consisting of

(a) the nucleotide sequence as set forth in SEQ ID NO: 7;

(b) a nucleotide sequence encoding the polypeptide as set forth in SEQ ID NO: 8;

(c) a nucleotide sequence which hybridizes under moderately or highly stringent conditions to the complement of (a) or (b), wherein the encoded polypeptide has an activity of the polypeptide as set forth in SEQ ID NO. 8, and

(d) a nucleotide sequence complementary to any of (a)-(c).

The invention also provides for an isolated nucleic acid molecule comprising a nucleotide sequence selected from the group consisting of.

(a) a nucleotide sequence encoding a polypeptide that is at least about 70, 75, 80, 85, 90, 95, 96, 97, 98, or 99 percent identical to the polypeptide as set forth in SEQ ID NO: 8 as determined using a computer program selected from the group consisting of GAP, BLASTP, BLASTN, FASTA, BLASTA, BLASTX, BestFit, and the Smith-Waterman algorithm, wherein the polypeptide has an activity of the polypeptide as set forth in SEQ ID NO: 8:

(b) a nucleotide sequence encoding an allelic variant or splice variant of the nucleotide sequence as set forth in SEQ ID NO: 7, wherein the encoded polypeptide has an activity of the polypeptide as set forth in SEQ ID NO: 8;

(c) a nucleotide sequence of SEQ ID NO: 7, (a), or (b) encoding, a polypeptide fragment of at least about 25 amino acid residues, wherein the polypeptide has an activity of the polypeptide as set forth in SEQ ID NO: 8;

(d) a nucleotide sequence encoding a polypeptide that has a substitution and/or deletion of 1 to 176 amino acid residues as set forth in any of SEQ ID NOS: 7-8 wherein the encoded polypeptide has an activity of the polypeptide as set forth in SEQ ID NO: 8;

(e) a nucleotide sequence of SEQ ID NO: 7, or (a)-(d) comprising a fragment of at least about 16 nucleotides;

(f) a nucleotide sequence which hybridizes under moderately or highly stringent conditions to the complement of any of (a)-(e), wherein the polypeptide has an activity of the polypeptide as set forth in SEQ ID NO: 8; and

(g) a nucleotide sequence complementary to any of (a)-(e).

The invention further provides for an isolated nucleic acid molecule comprising a nucleotide sequence selected from the group consisting of:

(a) a nucleotide sequence encoding a polypeptide as set forth in SEQ ID NO: 8 with at least one conservative amino acid substitution, wherein the polypeptide has an activity of the polypeptide as set forth in SEQ ID NO: 8;

(b) a nucleotide sequence encoding a polypeptide as set forth in SEQ ID NO: 8 with at least one amino acid insertion, wherein the polypeptide has an activity of the polypeptide as set forth in SEQ ID NO: 8;

(c) a nucleotide sequence encoding a polypeptide as set forth in SEQ ID NO: 8 with at least one amino acid deletion, wherein the polypeptide has an activity of the polypeptide as set forth in SEQ ID NO: 8;

(d) a nucleotide sequence encoding a polypeptide as set forth in SEQ ID NO: 8 which has a C- and/or N-terminal truncation, wherein the polypeptide has an activity of the polypeptide as set forth in SEQ ID NO. 8;

(e) a nucleotide sequence encoding a polypeptide as set forth in SEQ ID NO. 8 with at least one modification selected from the group consisting of amino acid substitutions, amino acid insertions, amino acid deletions, C-terminal truncation, and N-terminal truncation, wherein the polypeptide has an activity of the polypeptide as set forth in SEQ ID NO: 8;

(f) a nucleotide sequence of (a)-(e) comprising a fragment of at least about 16 nucleotides:

(g) a nucleotide sequence which hybridizes under moderately or highly stringent conditions to the complement of any of (a)-(f), wherein the polypeptide has an activity of the polypeptide as set forth in SEQ ID NO: 8; and

(h) a nucleotide sequence complementary to any of (a)-(e).

The invention also provides for an isolated polypeptide comprising, the amino acid sequence selected from the group consisting of:

(a) the mature amino acid sequence as set forth in SEQ ID NO: 8 comprising a mature amino terminus at residue(s) 1, and optionally further comprising an amino-terminal methionine;

(b) an amino acid sequence for an ortholog of SEQ ID NO: 8, wherein the encoded polypeptide has an activity of the polypeptide as set forth in SEQ ID NO: 8;

(c) an amino acid sequence that is at least about 70, 80, 85, 90, 95, 96, 97, 98, or 99 percent identical to the amino acid sequence of SEQ ID NO: 8 as determined using a computer program selected from the group consisting of GAP, BLASTP, BLASTN, FASTA, BLASTA, BLASTX, BestFit, and the Smith-Waterman algorithm, wherein the polypeptide has an activity of the polypeptide as set forth in SEQ ID NO: 8;

(d) a fragment of the amino acid sequence set forth in SEQ ID NO: 8 comprising at least about 25 amino acid residues, wherein the polypeptide has an activity of the polypeptide as set forth in SEQ ID NO: 8;

(e) an amino acid sequence for an allelic variant or splice variant of either the amino acid sequence as set forth in SEQ ID NO: 8 or at least one of (a)-(c) wherein the polypeptide has an activity of the polypeptide as set forth in SEQ ID NO: 8.

The invention further provides for an isolated polypeptide comprising the amino acid sequence selected from the group consisting of:

(a) the amino acid sequence as set forth in SEQ ID NO: 8 with at least one conservative amino acid substitution, wherein the polypeptide has an activity of the polypeptide as set forth in SEQ ID NO: 8;

(b) the amino acid seqeuence as set forth in SEQ ID NO: 8 with at least one amino acid insertion, wherein the polypeptide has an activity of the polypeptide as set forth in SEQ ID NO: 8;

(c) the amino acid sequence as set forth in SEQ ID NO: 8 with at least one amino acid deletion, wherein the polypeptide has an activity of the polypeptide as set forth in SEQ ID NO: 8;

(d) the amino acid sequence as set forth in SEQ ID NO: 8 which has a C-and/or N-terminal truncation, wherein the polypeptide has an activity of the polypeptide as set forth in SEQ ID NO: 8; and

(e) the amino acid sequence as set forth in SEQ ID NO: 8, with at least one modification selected from the group consisting of amino acid substitutions, amino acid insertions, amino acid deletions, C-terminal truncation, and N-terminal truncation, wherein the polypeptide has an activity of the polypeptide as set forth in SEQ ID NO: 8.

Also provided are fusion polypeptides comprising the amino acid sequences of (a)-(e) above.

The present invention also provides for all expression vector comprising the isolated nucleic acid molecules as set forth herein, recombinant host cells comprising recombinant nucleic acid molecules as set forth herein, and a method of producing a ymkz5-receptor polypeptide comprising culturing the host cells and optionally isolating the polypeptide so produced.

A transgenic non-human animal comprising a nucleic acid molecule encoding a ymkz5-receptor polypeptide is also encompassed by the invention. The ymkz5-receptor nucleic acid molecules are introduced into the animal in a manner that allows expression and increased levels of the ymkz5-receptor polypeptide, which may include increased circulating levels. The transgenic non-human animal is preferably a mammal.

Also provided are derivatives of the ymkz5-receptor polypeptides of the present invention.

Additionally provided are selective binding agents such as antibodies and peptides capable of specifically binding the ymkz5-receptor polypeptides of the invention. Such antibodies and peptides may be agnostic or antagonistic.

Pharmaceutical compositions comprising the nucleotides, polypeptides, or selective binding agents of the present invention and one or more pharmaceutically acceptable formulation agents are also encompassed by the invention. The pharmaceutical compositions are used to provide therapeutically effective amounts of the nucleotides or polypeptides of the present invention. The invention is also directed to methods of using the polypeptides, nucleic acid molecules, and selective binding agents. The invention also provides for devices to administer a ymkz75-receptor polypeptide encapsulated in a membrane.

The ymkz5-receptor polypeptide(s) of the invention and its biologically active variant(s), analog(s) and fragment(s) may be used for therapeutic and/or diagnostic purposes to treat, prevent and/or detect conditions resulting from the abnormal expression of ymkz5-receptor polypeptide or from the abnormal expression of a putative ymkz5-ligand or a member of the TNF family of ligands that binds to ymkz5-receptor polypeptide caused by overreaction of the host or deficiency of a natural autoregulatory network such as frequently observed in sepsis, cachexia, auto-immune responses, inflammatory diseases, viral, bacterial and parasitic diseases, and cancer.

The invention encompasses diagnosing a pathological condition or a susceptibility to a pathological condition in a subject caused by or resulting from abnormal levels of ymkz5-receptor polypeptide comprising determining the presence or amount of expression of the ymkz5-receptor polypeptide in a sample; and comparing the level of said polypeptide in a biological tissue or cellular sample from either normal subjects or the subject at an earlier time, wherein susceptibility to a pathological condition is based on the presence or amount of expression of the polypeptide.

The present invention also provides a method of assaying test molecules to identify a test molecule which binds to a ymkz5-receptor polypeptide. The method comprises contacting a ymkz5-receptor polypeptide with a test molecule and determining the extent of binding of the test molecule to the polypeptide. The method further comprises determining whether such test molecules are agonists or antagonists of a ymkz5-receptor polypeptide. The present invention further provides a method of testing the impact of molecules on the expression of ymkz5-receptor polypeptide or on the activity of ymkz5-receptor polypeptide.

Methods of regulating expression and modulating (i.e., increasing or decreasing) levels of a ymkz5-receptor polypeptide are also encompassed by the invention. One method comprises administering to an animal a nucleic acid molecule encoding a ymkz5-receptor polypeptide. In another method, a nucleic acid molecule comprising elements that regulate or modulate the expression of a ymkz5-receptor polypeptide may be administered. Examples of these methods include gene therapy, cell therapy, and anti-sense therapy as further described herein.

The ymkz5-receptor polypeptide can be used for identifying ligands thereof. Various forms of “expression cloning” have been used for cloning ligands for receptors. See e.g., Davis et al., Cell. 87:1161-1169 (1996). These and other ymkz5-receptor ligand cloning experiments are described in greater detail herein Isolation of the ymkz5-receptor ligand(s) allows for the identification or development of novel agonists and/or antagonists of the ymkz5-receptor signaling pathway. Such agonists and antagonists include ymkz5-receptor ligand(s), anti-ymkz5-receptor ligand antibodies and derivatives thereof, small molecules, or antisense oligonucleotides, any of which can be used for potentially treating one or more diseases or disorders, including those recited herein.

### DESCRIPTION OF THE FIGURES

FIGS. 1A and 1B presents an alignment of the predicted amino acid sequence of the full length ymkz5-receptor gene with the corresponding regions of the members of the TNF receptor super gene family, Fas a, and TNFR1. The alignment was performed using, GCG-Pileup program.

### DETAILED DESCRIPTION OF THE INVENTION

The section headings herein are for organizational purposes only and are not to be construed as limiting the subject matter described therein.

Definitions:

The term “ymkz5-receptor nucleic acid molecule” refers to a nucleic acid molecule comprising or consisting essentially of or comprising a nucleotide sequence as set forth in SEQ ID NO: 7, comprising or consisting essentially of a nucleotide sequence encoding the polypeptide as set forth in SEQ ID NO: 8, or nucleic acid molecules related thereto. Related nucleic acid molecules comprise or consist essentially of a nucleotide sequence that is about 70 percent identical to the nucleotide sequence as shown in SEQ ID NO: 7. or comprise or consist essentially of a nucleotide sequence encoding a polypeptide that is about 70 percent identical to the polypeptide as set forth in SEQ ID NO: 8. In preferred embodiments, the nucleotide sequences are about 75 percent, or about 80 percent, or about 85 percent, or about 90 percent, or about 95, 96, 97, 98, or 99 percent identical to the nucleotide sequence as shown in SEQ ID NO: 7, or the nucleotide sequences encode a polypeptide that is about 75 percent, or about 80 percent, or about 85 percent, or about 90 percent, or about 95, 96, 97, 98, or 99 percent identical to the polypeptide sequence as set forth in SEQ ID NO: 8. Related nucleic acid molecules also include fragments of the above ymkz5-receptor nucleic acid molecules which are at least about 10 contiguous nucleotides, or about 15, or about 20, or about 25, or about 50, or about 75, or about 100, or greater than about 100 contiguous nucleotides. Related nucleic acid molecules also include fragments of the above ymkz5-receptor nucleic acid molecules which encode a polypeptide of at least about 25 amino acid residues, or about 50, or about 75, or about 100, or greater than about 100 amino acid residues. Related nucleic acid molecules also include a nucleotide sequence encoding a polypeptide comprising or consisting essentially of a substitution and/or a deletion of one to 176 amino acid residues compared to the polypeptide in SEQ ID NO:8 Related ymkz5-receptor nucleic acid molecules include those molecules which comprise nucleotide sequences which hybridize under moderately or highly stringent conditions as defined herein with any of the above nucleic acid molecules. In preferred embodiments, the related nucleic acid molecules comprise sequences which hybridize under moderately or highly stringent conditions with the sequence as shown in SEQ ID NO.7, or with a molecule encoding a polypeptide comprising the sequence as shown in SEQ ID NO:8, or with a nucleic acid fragment as defined above, or with a nucleic acid fragment encoding a polypeptide as defined above. It is also understood that related nucleic acid molecules include allelic or splice variants of any of the above nucleic acids, and include sequences which are complementary to any of the above nucleotide sequences.

The term “nucleic acid sequence” or “nucleic acid molecule” refers to a DNA or RNA sequence. The term encompasses molecules formed from any of the known base analogs of DNA and RNA such as, but not limited to 4-acetylcytosine, 8-hydroxy-N6-methlyladenosine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term “naturally occurring” or “native” when used in connection with biological materials such as nucleic acid molecules, polypeptides, host cells, and the like, refers to materials which are found in nature and are not manipulated by man. Similarly, “non-naturally occurring” or “non-native” as used herein refers to a material that is not found in nature or that has been structurally modified or synthesized by man.

The term “isolated nucleic acid molecule” refers to a nucleic acid molecule of the invention that (1) has been separated from at least about 50 percent of proteins, lipids, carbohydrates or other materials with which it is naturally found when total DNA is isolated from the source cells, (2) is not linked to all or a portion of a polynucleotide to which the “isolated nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use.

The term “allelic variant” refers to one of several possible naturally occurring alternate forms of a gene occupying a given locus on a chromosome of an organism.

The term “splice variant” refers to nucleic acid molecule, usually RNA, which is generated by alternative processing of intron sequences in an RNA transcript.

The term “expression vector” refers to a vector which is suitable for propagation in a host cell and contains nucleic acid sequences which direct and/or control the expression of inserted heterologous nucleic acid sequences. Expression includes, but is not limited to, processes such as transcription, translation, and RNA splicing, if introns are present.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “pharmaceutically acceptable carrier” or “physiologically acceptable carrier” as used herein refers to one or more formulation materials suitable for accomplishing or enhancing the delivery of the ymkz5-receptor polypeptide, ymkz5-receptor nucleic acid molecule or ymk-receptor selective binding agent as a pharmaceutical composition.

The term “selective binding agent” refers to a molecule or molecules having specificity for an ymkz5-receptor polypeptide. As used herein, the terms, “specific” and “specificity” refer to the ability of the selective binding agents to bind to human ymkz5 like polypeptides and not to bind to human non-ymkz5 like polypeptides. It will be appreciated, however, that the selective binding agents may also bind orthologs of the polypeptide as set forth in SEQ ID NO: 8, that is, interspecies versions thereof, such as mouse and rat polypeptides.

The term “transduction” is used to refer to the transfer of genes from one bacterium to another, usually by a phage. “Transduction” also refers to the acquisition and transfer of eukaryotic cellular sequences by retroviruses.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, for example, Graham et al., Virology, 52:456 (1973); Sambrook et al., Molecular Cloning, a Laboratory Manual, Cold Spring Harbor Laboratories (New York, 1989); Davis et al., Basic Methods in Molecular Biology, Elsevier, (1986); and Chu et al., Gene, 13:197 (1981). Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, may be maintained transiently as an episomal element without being replicated, or may replicate independently as a plasmid. A cell is considered to have been stably transformed when the DNA is replicated with the division of the cell.

The term “host cell” is used to refer to a cell as which has been transformed, or is capable of being transformed with a nucleic acid sequence and then of expressing a selected gene of interest. The term includes the progeny of the parent cell, as whether or not the progeny is identical in morphology or in genetic make-up to the original parent, so long as the selected gene is present.

The term “ymkz5-receptor polypeptide” refers to a polypeptide comprising the amino acid sequence of SEQ ID NO: 8, and related polypeptides described herein. Related polypeptides includes allelic variants, splice variants, fragments, derivatives, substitution, deletion, and/or insertion variants, fusion polypeptides, and orthologs. ymkz5-receptor polypeptide(s) may be mature polypeptide(s), as defined herein, and may or may not have an amino terminal methionine residue, depending on the method by which they are prepared.

The term “ymkz5-receptor polypeptide fragment” refers to a peptide or polypeptide that comprises less than the full length amino acid sequence of a ymkz5-receptor polypeptide as set forth in SEQ ID NO: 8. Such a fragment may arise, for example, from a truncation at the amino terminus, a truncation at the carboxy terminus, and/or an internal deletion of the amino acid sequence. Ymkz5-receptor fragments may result from alternative RNA splicing or from in vivo protease activity. For transmembrane or membrane-bound forms of a ymkz5-receptor polypeptide, preferred fragments include soluble forms such as those lacking a transmembrane or membrane-binding domain.

The term “ymkz5-receptor polypeptide variants” refers to ymkz5-receptor polypeptides comprising amino acid sequences which contain one or more amino acid sequence substitutions, deletions (such as internal deletions and/or ymkz5-receptor fragmants), and/or additions (such as internal additions and/or ymkz5-receptor like fusion polypeptides) as compared to the ymkz5-receptor polypeptide amino acid sequence set forth in SEQ ID NO: 8. Variants may be naturally occurring (e.g., ymkz5-receptor allelic variants, ymkz-receptor orthologs and ymkz-receptor variants) or artificially constructed using recombinant DNA technology. Such ymkz5-receptor polypeptide variants may be prepared from the corresponding nucleic acid molecules encoding said variants, which have a DNA sequence that varies accordingly from the DNA sequences for wild type ymkz5-receptor polypeptide as set forth in SEQ ID NO: 7. In preferred embodiments, the variants have from 1 to 3, or from 1 to 5, or from 1 to 10, or from 1 to 15, or from 1 to 20, or from 1 to 25, or from 1 to 50, or from 1 to 75, or from 1 to 100, or more than 100 amino acid substitutions, insertions, additions and/or deletions, wherein the substitutions may be conservative, or non-conservative, or any combination thereof.

The term “ymkz5-receptor polypeptide fragment” refers to a polypeptide that comprises a truncation at the amino terminus (with or without a leader sequence) and/or a truncation at the carboxy terminus of the polypeptide as set forth in SEQ ID NO: 8, ymkz-receptor polypeptide allelic variants, ymkz5-receptor polypeptide orthologs, ymkz5-receptor polypeptide splice variants and/or an ymkz5-receptor polypeptide variant having one or more amino acid additions or substitutions or internal deletions (wherein the resulting polypeptide is at least 6 amino acids or more in length) as compared to the ymkz5-receptor polypeptide amino acid sequence set forth in SEQ ID NO: 8. ymkz5-receptor polypeptide fragments may result from alternative RNA splicing or from in vivo protease activity. For transmembrane or membrane-bound forms of an ymkz5-receptor polypeptide, preferred fragments include soluble forms such as those lacking a transmembrane or membrane-binding domain. In preferred embodiments, truncations comprise about 10 amino acids, or about 20 amino acids, or about 50 amino acids, or about 75 amino acids, or about 100 amino acids, or more than about 100 amino acids. The polypeptide fragments so produced will comprise about 25 contiguous amino acids, or about 50 amino acids, or about 75 amino acids, or about 100 amino acids, or about 150 amino acids, or about 200 amino acids. Such ymkz5-receptor polypeptide fragments may optionally comprise an amino terminal methionine residue. It will be appreciated that such fragments can be used, for example, to generate antibodies to ymkz5-receptor like polypeptides

The term “ymkz5-receptor fusion polypeptide” refers to a fusion of ymkz5-receptor polypeptide, fragment, variant, ortholag and/or derivative thereof, with one or more amino acids (such as heterologous peptide or polypeptide, preferably at the amino- or carboxy-terminus of the ymkz5-receptor polypeptide as set forth in SEQ ID NO: 8. A non-limiting example of such a fusion is a fusion between ymkz5-receptor polypeptide and the Fc fragment of an immunoglobulin molecule. Such fusion may serve to enhance stability either in vivo or in vivo (or both) of ymkz5-receptor. The heterologous polypeptide may also function to improve solubility, or improve circulatory half-life of ymkz5-receptor polypeptide or, for example, improve purification of the polypeptide.

The term “ymkz5-receptor polypeptide derivatives” refers to ymkz5-receptor polypeptides, variants, or fragments thereof, that have been chemically modified, for example, by covalent attachment of one or more water soluble polymers, N-linked or O-linked carbohydrates, sugars, phosphates, and/or other such molecules. Such modifications may be introduced into the molecule by reacting targeted amino acid residues of the purified or crude protein with an organic derivatizing agent that is capable of reacting with selected side chains or terminal residues. The resulting covalent derivatives are also useful in programs directed at identifying residues important for biological activity. The derivatives are modified in a manner that is different from naturally occurring ymkz5-receptor polypeptides, either in the type or location of the molecules attached to the polypeptide. Derivatives further include deletion of one or more chemical groups naturally attached to the ymkz5-receptor polypeptide.

The terms “biologically active ymkz5-receptor polypeptides,” “biologically active ymkz5-receptor polypeptide fragments,” “biologically active ymkz5-receptor polypeptide variants,” and “biologically active ymkz5-receptor polypeptide derivatives” refer to ymkz5-receptor polypeptides having at least one activity characteristic of a ymkz5-receptor polypeptide, such as the ability to bind and neutralize TNF-like ligand activity in biological assays. Immunogenic fragments of ymkz5-receptor polypeptide(s) are those capable of inducing an immune response in a host animal antibodies directed to the ymkz5-receptor fragment.

The term “isolated polypeptide” refers to a polypeptide of the present invention that (1) has been separated from at least about 50 percent of polynucleotides, lipids, carbohydrates or other materials with which it is naturally found when isolated from the source cell, (2) is not linked (by covalent or noncovalent interaction) to all or a portion of a polypeptide to which the “isolated polypeptide” is linked in nature, (3) is operably linked (by covalent or noncovalent interaction) to a polypeptide with which it is not linked in nature, or (4) does not occur in nature. Preferably, the isolated polypeptide is substantially free from any other contaminating polypeptides or other contaminants that are found in its natural environment that would interfere with its therapeutic, diagnostic, prophylactic or research use.

The term “ymkz5-receptor polypeptide ortholog” refers to a polypeptide from another species that corresponds to ymkz5-receptor polypeptide amino acid sequence as set forth in SEQ ID NO: 8. For example, mouse and human ymkz5-receptor polypeptides are considered orthologs of each other.

The term “mature ymkz5-receptor polypeptide” refers to a ymkz5-receptor polypeptide lacking a leader sequence, and may also include other modifications of a polypeptide such as proteolytic processing of the amino terminus (with or without a leader sequence) and/or the carboxy terminus, cleavage of a smaller polypeptide from a larger precursor, N-linked and/or O-linked glycosylation, and the like.

The term “mutein” refers to a mutant protein, polypeptide, variants, analogs or fragments of a ymkz5-receptor polypeptide. Muteins of ymkz5-receptor may be prepared by deletion, insertion, substitution, point mutation, truncation, addition, transposition, PCR amplification, site-directed mutagenesis or other methods known in the art.

The term “antitgen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The terms “effective amount” and “therapeutically effective amount” refer to the amount of a ymkz5-receptor polypeptide necessary to support an observable level of of one or more biological activities of the TNF-receptor polypeptides as set forth above, to bring about a meaningful patient benefit, i.e. treatment, healing, prevention, or amelioration of a condition. When applied to an individual active ingredient, administered alone, the term refers to that ingredient alone. When applied to combinations, the terms refers to combined amounts of active ingredients that result in therapeutic effect, when administered in combination, serially or simultaneously. The ymkz5-receptor polypeptides that have use in practicing the present invention may be naturally occuring full length polypeptides, truncated polypeptides, variant homologs, analogs, derivatives, or peptide fragments. Illustrative analogs include those in which one or more divergent amino acids between two species are substituted with the divergent amino acid from another species. Divergent amino acids may also be substituted with any other amino acid, whether it be a conservative or a non-conservative amino acid.

Relatedness of Nucleic Acid Molecules and/or Polypeptides

The term “identity”, as known in the art, refers to a relationship between the sequences of two or more polypeptide molecules or two or more nucleic acid molecules, as determined by comparing the sequences. In the art, “identity” also means the degree of sequence relatedness between polypeptide or nucleic acid molecules or polypeptides, as the case may be, as determined by the match between two or more strings of nucleotide or amino acid sequences. “identity” measures the percent of identical matches between two or more sequences with gap alignments, if any, addressed by a particular mathematical model or computer program (i.e., “algorithms”).

The term “similarity” is a related concept, but in contrast to “identity”, refers to a measure of similarity which includes both identical matches and conservative substitution matches. If two polypeptide sequences have, for example, 10/20 identical amino acids, and the remainder are all non-conservative substitutions, then the percent identity and similarity would both be 50%. If in the same example, there are 5 more positions where there are conservative substitutions, then the percent identity remains 50%, but the percent similarity would be 75% ( 15/20). Therefore, in cases where there are conservative substitutions, the degree of similarity between two polypeptide sequences will be higher than the percent identity between those two sequences.

It is understood that related nucleic acid molecules include allelic or splice variants of the nucleic acid molecule of SEQ ID NO: 8, and include sequences which are complementary to any of the above nucleotide sequences. Related nucleic acid molecules also include a nucleotide sequence encoding a polypeptide comprising or consisting essentially of a substitution, modification, addition and/or a deletion of one or more amino acid residues compared to the polypeptide in SEQ ID NO: 8.

Fragments include molecules which encode a polypeptide of at least about 25 amino acid residues, or about 50, or about 75, or about 100, or greater than about 100 amino acid residues of the polypeptide of SEQ ID NO: 8.

The term “highly stringent conditions” refers to those conditions that are designed to permit hybridization of DNA strands as whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015M sodium chloride, 0.0015M sodium citrate at 65-68° C. or 0.015M sodium chloride, 0.0015M sodium citrate, and 50% formamide at 42° C. See Sambrook, Fritsch & Maniatis, Molecular Cloning: A Laboratory Manual, 2nd Ed., Cold Spring Harbor Laboratory, (Cold Spring Harbor, N.Y. 1989); Anderson et al., Nucleic Acid Hybridisation: a practical approach, Ch. 4, IRL Press Limited (Oxford, England).

More stringent conditions (such as higher temperature, lower ionic strength, higher formamide, or other denaturing agent) may also be used, however, the rate of hybridization will be affected. Other agents may be included in the hybridization and washing, buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfalte (NaDodSO4 or SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: a Practical Approach, Ch. 4. IRL Press Limited (Oxford, England).

Factors affecting the stability of a DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex call be estimated by the following equation:

Tm(°C.)=81.5+16.6(log[Na+])+0.41(%G+C)−600/N−0.72(%formamide)

where N is the length of the duplex formed, [Na+] is the molar concentration of the sodium ion in the hybridization or washing solution, %G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015M sodium chloride, 0.0015M sodium citrate at 50-65° C. or 0.015M sodium chloride, 0.0015M sodium citrate, and 20% formamide at 37-50° C. By way of example, a “moderately stringent” condition of 50° C. in 0.015 M sodium ion will allow about a 21% mismatch.

It will be appreciated by those skilled in the art that there is no absolute distinction between “highly” and “moderately” stringent conditions. For example, at 0.015M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting temperature in 1M NaCl* for oligonucleotide probes up to about 20 nt is given by:

Tm=2° C. per A-┐ base pair+4° C. per G-C base pair

*The sodium ion concentration in 6×salt sodium citrate (SSC) is 1M. See Suggs et al.,

Developmental Biology Using Purified Genes, p. 683, Brown and Fox (eds.) (1981). High stringency washing conditions for oligonucleotide are usually at a temperature of 0-5° C. below the Tm of the oligonucleotide in 6×SSC, 0.1% SDS.

Differences in the nucleic acid sequence may result in conservative and/or non-conservative modifications of the amino acid sequence relative to the amino acid sequence of SEQ ID NO: 8.

The term “conservative amino acid substitution” refers to a substitution of a native amino acid residue with a nonnative residue such that there is little or no effect on the polarity or charge of the amino acid residue at that position. For example, a conservative substitution results from the replacement of a non-polar residue in a polypeptide with any other non-polar residue. Further, any native residue in the polypeptide may also be substituted with alanine, as has been previously described for “alanine scanning mutagenesis”. General rules for conservative amino acid substitutions are set forth in Table I.

TABLE I
Conservative Amino Acid Substitutions

| Basic: | Acidic: | Uncharged Polar: | Non-Polar: | |
|---|---|---|---|---|
| arginine | glutamic | glutamime | phenylalanine | valine |
| lysine | acid | asparagine | tryptophan | proline |
| histidine | aspartic | serine | cysteine | methionine |
| | acid | threonine | glycine | leucine |
| | | tyrosine | alanine | norleucine |
| | | | | isoleucine |

Conservative modifications to the amino acid sequence (and the corresponding modifications to the encoding nucleotides) are expected to produce ymkz5-receptors having functional and chemical characteristics similar to those of naturally occurring ymkz5-receptor. In contrast, substantial modifications in the functional and/or chemical characteristics of ymkz5-receptor may be accomplished by selecting substitutions that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

Naturally occurring residues may be divided into groups based on common side chain properties:

1) hydrophobic: norleucine, Met, Ala, Val, Leu, Ile;

2) neutral hydrophilic: Cys, Ser, Thr;

3) acidic: Asp, Glu;

4) basic: Asn, Gln, His, Lys, Arg;

5) residues that influence chain orientation: Gly, Pro, and

6) aromatic: Trp, Tyr, Phe.

Non-conservative substitutions may involve the exchange of a member of one of these classes for a member from another class. Such substituted residues may be introduced into regions of the human ymkz5-receptor molecule that are homologous with non-human ymkz5-receptor or into the non-homologous regions of the molecule.

Conservative amino acid substitutions also encompass non-naturally occurring amino acid residues which are typically incorporated by chemical peptide synthesis rather than by synthesis in biological systems. These include peptidomimetics and other reversed or inverted forms of amino acid moieties.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenic and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0 4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred. One may also identify epitopes from primary amino acid sequences on the basis of hydrophilicity. These regions are also referred to as “epitopic core regions.”

Desired amino acid substitutions (whether conservative or non-conservative) can be determined by those skilled in the art at the time such substitutions are desired. For example, amino acid substitutions can be used to identify important residues of the ymkz5-receptor polypeptide, or to increase or decrease the affinity of the ymkz5-receptor polypeptides described herein.

Exemplary amino acid substitutions are set forth in Table II.

TABLE II
Conservative Amino Acid Substitutions

| | | |
|---|---|---|
| Original | | Preferred |
| Residues | Exemplary Substitutions | Substitutions |

| | | |
|---|---|---|
| Ala | Val, Leu, Ile | Val |
| Aru | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of the polypeptide as set forth in SEQ ID NO: 8 using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of an ymkz5-receptor polypeptide to such similar polypeptides. With such a comparison, one can identify residues and portions of the molecules that are conserved among similar polypeptides. It will be appreciated that changes in areas of an ymkz5-receptor like polypeptide that are not conserved relative to such similar polypeptides would be less likely to adversely affect the biological activity and/or structure of the ymkz5-receptor polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in an ymkz5-receptor polypeptide that correspond to amino acid residues that are important for activity or structure in similar polypeptides. One skilled in the art may opt for chemically similar amino acid substitutions for such predicted important amino acid residues of ymkz5-receptor polypeptides.

One skilled in the art can also analyze the three-dimensional structure and amino acid sequence in relation to that structure in similar polypeptides. In view of that information, one skilled in the art may predict the alignment of amino acid residues of an ymkz5-receptor polypeptide with respect to its three dimensional structure. One skilled in the art may choose not to make radical chances to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays know to those skilled in the art. Such variants could be used to gather information about suitable variants. For example, if one discovered that a chance to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult. Curr Opin in Biotech., 7(4):422-427 (1996). Chou et al , Biochemistry, 13(2):222-245 (1974); Chou et al., Biochemistry, 113 (2):211-222 (1974); Chou et al.,Adv. Enzymol. Relat. Areas Mol Biol., 47:45-148 (1978); Chou et al., Ann. Rev. Biochem., 47:251-276 (1978); and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Opin. Struct. Biol., 7(3):369-376 (1997)) that there are a limited number of folds in a given polypeptide or protein and that once a critical number of structures have been resolved, structural prediction will gain dramatically in accuracy.

Additional methods of predicting secondary structure include “threading” (Jones, D., Curr. Opin. Struct. Biol., 7(3):377-87 (1997); Sippl et al., Structure, 4(1):15-9 (1996)), “profile analysis” (Bowie et al , Science, 253:164-170 (1991); Gribskov et (al., Meth. Enzym., 183:146-159 (1990): Gribskov et al., Proc. Nat. Acad. Sci. U.S.A., 84(13):4355-4358 (1987)), and “evolutionary linkage” (See Home, supra, and Brenner, supra).

Preferred ymkz5-receptor polypeptide variants include glycosylation variants wherein the number and/or type of glycosylation sites has been altered compared to the amino acid sequence set forth in SEQ ID NO: 8. In one embodiment, ymkz5 like polypeptide variants comprise a greater or a lesser number of N-linked glycosylation sites than the amino acid sequence set forth in SEQ ID NO: 8. An N-linked glycosylation site is characterized by the sequence: Asn-X-Ser or Asn-X-Thr, wherein the amino acid residue designated as X may be any amino acid residue except proline. The substitution(s) of amino acid residues to create this sequence provides a potential new site for the addition of an N-linked carbohydrate chain.

Alteratively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred ymkz5-receptor variants include cysteine variants, wherein one or more cysteine residues are deleted from or substituted for another amino acid (e.g., serine) as compared to the amino acid sequence set forth in SEQ ID NO: 8. Cysteine variants are useful when ymkz5-receptor polypeptides must be refolded into a biologically active conformation such as after the isolation of insoluble inclusion bodies. Cysteine variants generally have fewer cysteine residues than the native protein, and typically have an even number to minimize interactions resulting from unpaired cysteines.

In addition, the polypeptide comprising the amino acid sequence of SEQ ID NO: 8 or an ymkz5-receptor polypeptide variant may be fused to a homologous polypeptide to form a homodimer or to a heterologous polypeptide to form a heterodimer. Heterologous peptides and polypeptides include, but are not limited to: an epitope to allow for the detection and/or isolation of an ymkz5-receptor fusion polypeptide; a transmembrane receptor protein or a portion thereof, such as an extracellular domain, or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region; and a polypeptide which has a therapeutic activity different from the polypeptide comprising the amino acid sequence as set forth in SEQ ID NO: 8 or an ymkz-receptor polypeptide variant.

Fusions can be made either at the amino terminus or at the carboxy terminus of the polypeptide comprising the amino acid sequence set forth in SEQ ID NO: 8 or an ymkz5-receptor polypeptide variant. Fusions may be direct with no linker or adapter molecule or indirect using a linker or adapter molecule. A linker or adapter molecule may be one or more amino acid residues, typically up to about 20 to about 50 amino acid residues. A linker or adapter molecule may also be designed with a cleavage site for a DNA restriction endonuclease or for a protease to allow for the separation of the fused moieties It will be appreciated that once constructed, the fusion polypeptides can be derivatized according to the methods described herein.

In a further embodiment of the invention, the polypeptide comprising the amino acid sequence of SEQ ID NO: 8 or an ymkz5-receptor polypeptide variant is fused to one or more domains of an Fc region of human IgG. Antibodies comprise two functionally independent parts, a variable domain known as “Fab”, which binds antigen, and a constant domain known as “Fc”, which is involved in effector functions such as complement activation and attack by phagocytic cells. An Fc has a long serum half-life, whereas an Fab is short-lived. Capon et al., Nature, 337:525-31 (1989). When constructed together with a therapeutic protein, an Fc domain can provide longer half-life or incorporate such functions as Fc receptor binding, protein A binding, complement fixation and perhaps even placental transfer. Id. Table II summarizes the use of certain Fc fusions known in the art.

TABLE II
Fc Fusion with Therapeutic Proteins

| Form of | Fusion | Therapeutic | |
|---|---|---|---|
| Fc | partner | implications | Reference |
| IgG1 | N-terminus of | Hodgkin's | U.S. Pat. No. |
| | CD30-L | disease; | 5,480,981 |
| | | anaplastic | |
| | | lymphoma; | |
| | | T-cell leukemia | |
| Murine | IL-10 | anti- | Zheng et al. (1995). |
| Fcg2a | | inflammatory; | J. Immunol., 154 |
| | | transplant | 5590-5600 |
| | | rejection | |
| IgG1 | TNF receptor | septic shock | Fisher et al. (1996). |
| | | | N. Engl J. Med. |
| | | | 334: 1697-1702; |
| | | | Van Zee et al., |
| | | | (1996), J. Immunol., |
| | | | 2221-2330 |
| IgG, IgA, | TNF receptor | inflammation, | U.S. Pat. No. |
| IgM, or IgE | | autoimmune | 5,808,029, issued |
| (excluding | | disorders | Sep. 15, 1998 |
| the first | | | |
| domain) | | | |
| IgG1 | CD4 receptor | AIDS | Capon et al. (1989), |
| | | | Nature 337: 525-531 |
| IgG1, | N-terminus | anti-cancer, | Harvill el al. |
| IgG3 | of IL-2 | antiviral | (1995). |
| | | | Immunotech., 1: 95-105 |
| IgG1 | C-terminus of | osteoarthritis; | WO 97/23614, |
| | OPG | bone density | published Jul. 3, |
| | | | 1997 |
| IgG1 | N-terminus of | ann-obesity | PCT/US 97/23183, |
| | leptin | | filed Dec. 11, 1997 |
| Human Ig | CTLA-4 | autoimmune | Linsley (1991), |
| Cg1 | | disorders | J. Exp. Med., |
| | | | 174:561-569 |

In one example, all or a portion of the human IgG hinge, CH2 and CH3 regions may be fused at either the N-terminus or C-terminus of the ymkz5-receptor polypeptides using methods known to the skilled artisan. The resulting ymkz5-receptor fusion polypeptide may be purified by use of a Protein A affinity column. Peptides and proteins fused to an Fc region have been found to exhibit a substantially greater half-life in vivo than the unfused counterpart. Also, a fusion to an Fc region allows for dimerization/multimerization of the fusion polypeptide. The Fc region may be a naturally occurring Fc region, or may be altered to improve certain qualities, such as therapeutic qualities, circulation time, reduce aggregation. etc.

Identity and similarity of related nucleic acid molecules and polypeptides can be readily calculated by known methods. Such methods include, but are not limited to, those described in Computational Molecular Biology, Lesk, A. M., ed., Oxford University Press, (New York, 1988); Biocomputing: Informatics and Genome Projects, Smith, D. W., ed., Academic Press. (New York, 1993); Computer Analysis of Sequence Data, Part 1, Griffin, A. M., and Griffin., H. G., eds., Humana Press, (New Jersey, 1994); Sequence Analysis in Molecular Biology, von Heinje, G., Academic Press. (1987); Sequence Analysis Primer, Gribskov M. and Devereux, J., eds., M. Stockton Press, New York, 1991; and Carillo et al., SIAM J. Applied Math., 48:1073 (1988).

Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., Nucleic Acids Research 12(1):387 (1984); Genetics Computer Group, University of Wisconsin, Madison, Wis.), BLASTP, BLASTN, and FASTA (Atschul et al., J. Molec. Biol. 215:403-410 (1990). The BLAST X program is publicly available from the National Center for Biotechnology Information (NCBI) and other sources (BLAST Manual, Altschul et al. NCB NLM NIH Bethesda, Md. 20894; Altschul et al , J. Mol. Biol. 215:403-410 (1990). The well known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full length sequences. Accordingly, in preferred embodiment, the selected alignment method (GAP program) will result in an alignment that spans at least 50 contiguous amino acids of the target polypeptide.

For example, using the computer algorithm GAP (Genetics Computer Group. University of Wisconsin, Madison, Wis.). two polypeptides for which the percent sequence identity is to be determined are aligned for optimal matching of their respective amino acids (the “matched span”, as determined by the algorithm). A gap opening penalty (which is calculated as 3×the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al , in: Atlas of Protein Sequence and Structure, vol. 5, supp.3 ( 1978) for the PAM 250 comparison matrix; see Henikoff et al., Proc. Natl. Acad. Sci USA, 89:10915-10919 (1992)] for the BLOSUM 62 comparison matrix) is also used by the algorithm.

Preferred parameters for a polypeptide sequence comparisons include the following:

Algorithm: Needleman and Wunsch, J. Mol. Biol. 48:443-453 ( 1970),

Comparison matrix: BLOSUM 62 from Henikoff and Henikoff, Proc. Natl. Acad. Sci. USA 89:10915-10919 (1992).

Gap Penalty: 12

Gap Length Penalty: 4

Threshold of Similarity: 0

The GAP program Is useful with the above parameters. The aforementioned parameters are the default parameters for polypeptide comparisons (along with no penalty for end gaps) using the GAP algorithm

Preferred parameters for nucleic acid molecule sequence comparison include the following:

Algorithm: Needleman and Wunsch, J. Mol Biol. 48:443-453 (1970)

Comparison matrix: matches=+10, mismatch=0

Gap Penalty: 50

Gap Length Penalty: 3

The GAP program is also useful with the above parameters. The aforementioned parameters are the default parameters for nucleic acid molecule comparisons.

Other exemplary algorithms, gap opening penalties, gap extension penalties, comparison matrices, thresholds of similarity, etc. may be used by those of skill in the art, including those set forth in the Program Manual, Wisconsin Package, Version 9, September 1997. The particular choices to be made will depend on the specific comparison to be made, such as DNA-to DNA, protein-to-protein, protein-to-DNA; and additionally, whether the comparison is between given pairs of sequences (in which case GAP or BestFit are generally preferred) or between one sequence and a large database of sequences (in which case FASTA or BLASTA are preferred).

Certain alignment schemes for aligning two amino acid sequences may result in matching of only a short region of the two sequences, and this small aligned region may have very high sequence identity even though there is no significant relationship between the two full length sequences. Accordingly, in a preferred embodiment, the selected alignment method will result in an alignment that spans at least about 58 contiguous amino acids of the claimed full length polypeptide.

Synthesis

It will be appreciated by those skilled in the art the nucleic acid and polypeptide molecules described herein may be produced by recombinant and other means.

Nucleic Acid Molecules

The nucleic acid molecules encode a polypeptide comprising, the amino acid sequence of an ymkz5-receptor polypeptide can readily be obtained in a variety of ways including, without limitation, chemical synthesis, cDNA or genomic library screening, expression library screening and/or PCR amplification of cDNA.

Recombinant DNA methods used herein are generally, but not limited to, those set forth in Sambrook et al. (Molecular Cloning: A Laboratory Manual, Cold Spring Harbor Laboratory Press, Cold Spring Harbor, N.Y. (1989)) and/or Ausubel et al , eds., (Current Protocols in Molecular Biology, Green Publishers Inc. and Wiley and Sons, NY (1994)).

The present invention provides for nucleic acid molecules as described herein and methods for obtaining the molecules. A gene or cDNA encoding a “ymkz5-receptor polypeptide” or fragment thereof may be obtained by hybridization screening of a genomic or cDNA library, or by PCR amplification. Probes or primers useful for screening a library by hybridization can be generated based on sequence information for other known genes or gene fragments from the same or a related family of genes, such as, for example, conserved motifs. In addition, where a gene encodings ymkz5-receptor polypeptide has been identified from one species, all or a portion of that gene may be used as a probe to identify corresponding genes from other species (orthologs) or related genes from the same species (homologs). The probes or primers may be used to screen cDNA libraries from various tissue sources believed to express the “ymkz5-receptor gene.”

In addition, part or all of a nucleic acid molecule having the sequence as set forth in SEQ ID NO: 7 may be used to screen a genomic library to identify and isolate a gene encoding a “ymkz5-receptor.” Typically, conditions of moderate or high stringency will be employed for screening to minimize the number of false positives obtained from the screen.

The availability of the cDNA encoding the ymkz5-receptor, or fractions thereof, is the prerequisite for obtaining the genomic DNA. Under stringent conditions, a DNA library is screened and the clones obtained are investigated to see whether they contain the regulatory sequence elements needed for gene expression in addition to the coding, regions (e g checking for promoter function by fusion with coding regions of suitable reporter genes). Methods for screening DNA libraries under stringent conditions are taught, for example, in EPO 0 174 143, incorporated herein by reference. Obtaining the genomic DNA sequence makes it possible to investigate the regulatory sequences situated in the area which does not code for the “ymkz5-receptor”, particularly in the 5′-flanking region, for any possible interaction with known substances which modulate gene expression, e.g. transcription factors or steroids, or possibly discover new substances which might have a specific effect on the expression of this gene. The results of such investigations provide the basis for the targeted use of such substances for modulating ymkz5-receptor expression and, hence, for directly influencing the ability of the cells to interact with TNF family of ligands. As a result, the specific reaction with the ligands and the resulting effects can be suppressed.

The scope of the present invention also includes DNAs which code for subtypes of the ymkz5-receptor or its soluble forms, which may have properties different from those of the present ymkz5-receptor. These are expression products which are formed by alternative splicing and have modified structures in certain areas e.g. structures which can bring about a change in the affinity and specificity for the ligand or a change in terms of the nature and efficiency of signal transmission.

With the aid of the cDNA coding for the ymkz5-receptor it is possible to obtain nucleic acids which hybridize faith the cDNA or fragments thereof under conditions of low stringency and code for a polypeptide capable of binding TNF-related ligands or contain the sequence coding for such a polypeptide.

According to a further aspect, the invention relates to recombinant ymkz5-receptor polypeptide(s), preferably in a secretable form which constitutes the soluble part of the ymkz5-receptor. The invention also contemplates the production of a soluble form of the ymkz5-receptor, which is secreted into the cell supernatant, by recombinant DNA technology wherein the DNA coding for ymkz5-receptor, with a sequence coding for a signal peptide under the control of a suitable promoter, is introduced into suitable host organisms, especially eukaryotic and preferably higher eukaryotic cells.

Nucleic acid molecules encoding ymkz5-receptor polypeptides may also be identified by expression cloning which employs the detection of positive clones based upon a property of the expressed protein. Typically, nucleic acid libraries are screened by binding of an antibody or other binding partner (e.g., receptor or ligand) to cloned proteins which are expressed and displayed on a host cell surface. The antibody or the binding partner is modified with a detectable label to identify those cells expressing the desired clone.

Two murine TNF receptors, ymkz5-receptor described herein, and tmst2-receptor (cloned by Amgen), have been identified to be closely linked within the murine genome. Both of these novel receptors bind TRAIL in a species specific manner (See Example 8). Therefore, the characterization of the murine genes, ymkz5 and tmst2, may aid in the discovery of human TRAIL decoy receptors based on functionality and not solely based on primary sequence homology. Identification of human ymkz5/tmst2 orthologs of the invention will be facilitated by chromosomal and structural studies to reveal two highly related genes which are closely linked on the chromosome, one which is GPI-linked and the other a transmembrane receptor. Alternatively, the human genome may only harbor one ortholog which may be identified in the region syntenic with the mouse ymkz5/tmst2 locus.

Recombinant expression techniques conducted in accordance with the descriptions set forth below may be followed to produce these polynucleotides and to express the encoded polypeptides. For example, by inserting a nucleic acid sequence which encodes the amino acid sequence of an ymkz5-receptor polypeptide into an appropriate vector, one skilled in the art can readily produce large quantities of the desired nucleotide sequence. The sequences can then be used to generate detection probes or amplification primers. Alternatively, a polynucleotide encoding the amino acid sequence of an ymkz5-receptor polypeptide can be inserted into an expression vector. By introducing the expression vector into an appropriate host, the encoded ymkz5-receptor polypeptide may be produced in large amounts.

Another method for obtaining a suitable nucleic acid sequence is the polymerase chain reaction (PCR). In this method, cDNA is prepared from poly(A)+RNA or total RNA using the enzyme reverse transcriptase. Two primers, typically complementary to two separate regions of cDNA (oligonucleotides) encoding the amino acid sequence of an ymkz5-receptor polypeptide, are then added to the cDNA along with a polymerase such as Taq polymerase, and the polymerase amplifies the cDNA region between the two primers.

Another means of preparing a nucleic acid molecule encoding a variant ymkz5-receptor polypeptide, or a biologically active fragment thereof, is by chemical synthesis using methods well known to the skilled artisan such as those described by Engels et al (Angew Chem. Intl. Ed., 28:716-734 (1989)). These methods include, inter alia, the phosphotriester, phosphoramidite, and H-phosphonate methods for nucleic acid synthesis. A preferred method for such chemical synthesis is polymer-supported synthesis using standard phosphoramidite chemistry. Typically, the DNA encoding the ymkz5-receptor polypeptide will be several hundred nucleotides in length. Nucleic acids larger than about 100 nucleotides can be synthesized as several fragments using these methods. The fragments can then be ligated together to form the full length ymkz5-receptor polypeptide. Usually, the DNA fragment encoding the amino terminus of the polypeptide will have an ATG, which encodes a methionine residue. This methionine may or may not be present on the mature form of the ymkz5-receptor polypeptide, depending on whether the polypeptide produced in the host cells is designed to be secreted from that cell..

In some cases, it may be desirable to prepare nucleic acid molecules encoding ymkz5-receptor polypeptide variants or muteins. Nucleic acid molecules encoding variants may be produced using site directed mutagenesis, transposition. deletion, addition, truncation, PCR amplification, or other appropriate methods, where the primer(s) have the desired point mutations (see Sambrook et al , supra, and Ausubel et al., supra, for descriptions of mutagenesis techniques), provided that DNA's modified in this way code for polypeptides capable of binding one or more members of the TNF-family. Chemical synthesis using methods described by Engels et al., supra, may also be used to prepare such variants. Other methods known to the skilled artisan may be used as well.

In certain embodiments, nucleic acid variants contain codons which have been altered for the optimal expression of a ymkz5-receptor polypeptide in a given host cell. Particular codon alterations will depend upon the ymkz5-receptor polypeptide(s) and host cell(s) selected for expression. Such “codon optimization” can be carried out by a variety of methods, for example, by selecting codons which are preferred for use in highly expressed genes in a given host cell. Computer algorithms which incorporate codon frequency tables such as “Ecohigh. cod” for codon preference of highly expressed bacterial genes may be used and are provided by the University of Wisconsin Package Version 9.0, Genetics Computer Group, Madison, Wis. Other useful codon frequency tables include “Celegans_high.cod”, “Celegans_low.cod”, “Drosophila_high.cod”, “Human_high.cod”, “Maize_high.cod”, and “Yeast_high.cod”.

In other embodiments, nucleic acid molecules encode ymkz5-receptor variants with conservative amino acid substitutions as defined above, ymkz5-receptor variants comprising an addition and/or a deletion of one or more N-linked or O-linked glycosylation sites, or ymkz5-receptor polypeptide fragments as described above In addition, nucleic acid molecules may encode any combination of ymkz5-receptor variants, fragments, and fusion polypeptides described herein provided that DNA's modified in this way code for polypeptides capable of finding one or more members of TNF super gene family of ligands and receptors.

Expression of ymkz5 in Eukaryotic and Prokaryotic Cells

A nucleic acid molecule encoding a ymkz5-receptor polypeptide may be inserted into an appropriate expression vector using standard ligation techniques. The vector is typically selected to be functional in the particular host cell employed (i e., the vector is compatible with the host cell machinery such that amplification of the gene and/or expression of the gene can occur). A nucleic acid molecule encoding a ymkz5-receptor polypeptide may be amplified/expressed in prokaryotic, yeast, insect (baculovirus systems) and/or eukaryotic host cells. Selection of the host cell will depend in part on whether the ymkz5-receptor polypeptide is to be post-transitionally modified (e.g, glycosylated and/or phosphorylated). If so, yeast, insect, or mammalian host cells are preferable. For a review of expression vector, see Meth. Enz. vol.185, D. V. Goeddel ed. Academic Press Inc. San Diego, Calif. ( 1990).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotides: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker reunion for inserting the nucleic acid encoding the polypeptide to be expressed, and a selectable marker element. Each of these sequences is discussed below.

Optionally, the vector may contain a “tag” sequence, i.e., an oligonucleotide molecule located at the 5′ or 3′ end of the ymkz5-receptor polypeptide coding sequence, the oligonucleotide molecule encodes polyHis (such as hexaHis), or other “tag” such as FLAG, HA (hemaglutinin Influenza virus) or myc for which commercially available antibodies exist. Optionally, the ymkz5 gene can also be fused in frame at the N-terminal for example to an IgG Fc region. This tag is typically fused to the polypeptide upon expression of the polypeptide, and can serve as means for affinity purification of the ymkz5-receptor polypeptide from the host cell although it may also prolong the circulatory of a ymkz5-receptor polypeptide. Affinity purification can be accomplished, for example, by column chromatography using antibodies or protein-A column against the tag as an affinity matrix. Optionally, the tag can subsequently be removed from the purified ymkz5-receptor polypeptide by various means such as using certain peptidases for cleavage.

The 5′-flanking region of a gene contains a nucleic acid sequence to which RNA polymerase binds and initiates transcription. This nucleic sequences, known as the promoter region, determines both the nature of the enzyme that attaches to it and the rate of RNA synthesis. A number of eukaryotic and prokaryotic promoter elements are known in the art and are used to enhance gene transcription. Falnking sequences may be homologous (i.e., from the same species and/or strain as the host cell), heterologous (i.e, from a species other than the host cell species or strain), hybrid (i.e., a combination of flanking sequences from more than one source), or synthetic, or the flanking sequences may be native sequences which normally function to regulate ymkz5-receptor expression. As such, the source of a flanking sequence may be any prokaryotic or eukaryotic organism, any vertebrate or invertebrate organism, or any plant, provided that the flanking sequences is functional in, and can be activated by, the host cell machinery.

The flanking sequences useful in the vectors of this invention may be obtained by any of several methods well known in the art. Typically, flanking promoter sequences useful herein other than the endogenous ymkz5-receptor gene flanking sequences will have been previously identified by mapping and/or by restriction endonuclease digestion and can thus be isolated from the proper tissue source using the appropriate restriction endonucleases. In some cases, the full nucleotide sequence of one or more flanking sequence may be known Here, the flanking sequence may be synthesized using the methods described above for nucleic acid synthesis or cloning.

Where all or only a portion of the flanking sequence is known, it may be obtained using PCR and/or by screening a genomic library with suitable oligonucleotide and/or flanking sequence fragments from the same or another species.

Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origen aids in the amplification of the vector in a host cell. Amplification of the vector to a certain copy number can, in some cases, be important for the optimal expression of the ymkz5-receptor polypeptide. If the vector of choice does not contain an origin of replication site, one may be chemically synthesized based on a known sequence, and ligated into the vector. For example, the origin of replication from the plasmid pbr322 (Product No. 303-3s, New England Biolabs, Beverly, Mass.) is suitable for most Gram-negative bacteria and various origins (e.g., SV 40, polyoma, adenovirus, vesicular stomatitis virus (VSV) or papilloma viruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide coding region and serves to terminate transcription. Usually, a transcription termination sequence in prokaryotic cells is a G-C rich fragment followed by a poly T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene which will be expressed. Amplification is the process wherein genes which are in greater demand for the production of a protein critical for growth are reiterated in tandem within the chromosomes of successive generations of recombinant cells. Examples of suitable, amplifiable, selectable markers for mammalian cells include dihydrofolate reductase (DHFR) and thymidine kinase. The mammalian cell transformants are placed under selection pressure which only the transformants are uniquely adapted to survive by virtue of the marker present in the vector. Selection pressure is imposed by culturing the transformed cells under conditions in which the concentration of selection agent in the medium is successively changed, thereby leading to the amplification of both the selection gene and the DNA that encodes ymkz5-receptor. As a result, increased quantities of ymkz5-receptor polypeptide are synthesized from the amplified DNA.

A ribosome binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the ymkz5-receptor polypeptide to be expressed. The Shine-Dalgarno sequence is varied but is typically a polypurine (i e , having a high A-G content). Many Shine-Dalgarno sequences have been identified, each of which can be readily synthesized using methods set forth above and used in a prokaryotic vector.

A leader, or signal, sequence may be used to direct the secretion of ymkz5-receptor polypeptide out of the host cell where it is synthesized. Typically, the signal sequence is positioned in the coding region of the ymkz5-receptor nucleic acid molecule, or directly at the 5′ end of the ymkz5-receptor polypeptide coding region. Many signal sequences have been identified, and any of those that are functional in the selected host cell may be used in conjunction with the ymkz5-receptor gene or cDNA. Therefore, a signal sequence may be homologous (naturally occurring) or heterologous to the ymkz/5-receptor gene or cDNA, and may be homologous or heterologous to the ymkz5-receptor gene or cDNA. Additionally, a signal sequence may be chemically synthesized using methods described herein.. In most cases, the secretion of an ymkz5-receptor polypeptide from the host cell via the presence of a signal peptide will result in the removal of the signal peptide from the ymkz5-receptor polypeptide.

The signal sequence may be a component of the vector, or it may be a part of ymkz5-receptor DNA that is inserted into the vector. The native ymkz5-receptor DNA encodes a signal sequence at the amino terminus of the protein that is cleaved during post-translational processing of the molecule to form the mature ymkz5-receptor protein product. Included within the scope of this invention are ymkz5-receptor nucleotides with the native signal sequence as well as ymkz5-receptor nucleotides wherein the native signal sequence is deleted and replaced with a heterologous signal sequence. The heterologous signal sequence selected should be one that is recognized and processed, i.e., cleaved by a signal peptidase, by the host cell. For prokaryotic host cells that do not recognize and process the native ymkz5-receptor signal sequence, the signal sequence is substituted by a prokaryotic signal sequence selected, for example, from the group of the alkaline phosphatase, penicillinase, or heat-stable enterotoxin II leaders. For yeast secretion, the native ymkz5-receptor signal sequence may be substituted by the yeast iuvertase, alpha factor, or acid phosphates signal sequences. For mammalian cell expression the native signal sequence of the ymkz5-receptor polypeptide is satisfactory, although other mammalian signal sequences may be suitable.

In some cases, such as where glycosylation is desired in a eukaryotic host cell expression system, one may manipulate the various presequences to improve glycosylation or yield. For example, one may alter the peptidase cleavage site of a particular signal peptide, or add presequences, which also may affect glycosylation. The final protein product may have, in the -1 position (relative to the first amino acid of the mature protein) one or more additional amino acid residues incident to expression, which may not have been totally removed. For example, the final protein product may have one or two amino acids found in the peptidase cleavage site, attached to the N-terminus Alternatively, use of some enzyme cleavage sites may result in a slightly truncated from of the desired ymkz5-receptor polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns may be naturally occurring within the ymkz5-receptor gene, especially where the gene used is a full length genomic sequence or a fragment thereof. Where the intron is not naturally occurring within the gene (as for most cDNAs), the intron(s) may be obtained from another source. The position of the intron with respect to 5′-flanking sequences and the ymkz5-receptor gene is generally important, as the intron must be transcribed to be effective. Thus, when an ymkz5-receptor cDNA molecule is being expressed, the preferred position for the intron is 3′ to the transcription start site, and 5′ to the polyA transcription termination sequence. Preferably, the intron or introns will be located on one side or the other (i.e., 5′ or 3′) of the cDNA such that it does not interrupt the this coding sequence. Any intron from any source, including any viral, prokaryotic and eukaryotic (plant or animal) organisms, may be used to practice this invention, provided that it is compatible with the host cell(s) into which it is inserted. Also included herein are synthetic introns. Optionally, more than one intron may be used in the vector.

The expression and cloning vectors of the present invention will typically contain a promoter that is recognized by the host organism and operably linked to the molecule encoding the ymkz5-receptor protein.

Promoters are untranslated sequences located upstream (5′ to the start codon of a structural gene (generally within about 100 to 1000 bp) that control the transcription and translation of a molecule, such as that encoding ymkz-receptor. Promoters are conventionally grouped into one of two classes, inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product productions that is; there is little or no control over gene expression. A large number of promoters, recognized by a variety of potential host cells, are well known. A suitable promoter is operably linked to the DNA encoding ymkz5-receptor by removing the promoter from the source DNA by restriction enzyme digestion and inserting the desired promoter sequence into the vector. The native ymkz5-receptor promoter sequence may be used to direct amplification and/or expression of ymkz5-receptor encoding DNA. A heterologous promoter is preferred, however, if it permits greater transcription and higher yields of the expressed protein as compared to the native promoter, and if it is compatible with the host cell system that has been selected for use.

Promoters suitable for use with prokaryotic hosts include, but are not limited to the beta-lactamase and lactose promoter systems; alkaline phosphatase, a tryptophan (trp) promoter system; and hybrid promoters such as the tac promoter. Other known bacterial promoters are also suitable. Their sequences have been published, thereby enabling one skilled in the art to ligate them to the desired DNA sequence(s), using linkers or adapters as needed to supply any required restriction sites.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus, herpes virus and most preferably Simian Virus 40 (SV40). Other suitable mammalian promoters include heterologous mammalian promoters, e.g., heat-shock promoters and the actin promoter.

Additional promoters which may be of interest in controlling ymkz5 expression include, but are not limited to, the SV40 early promoter region (Bernoist and Chambon, Nature, 290:304-310, (1981)); the CMV promoter; the promoter contained in the 3′ long terminal repeat (LTR) of Rous sarcoma virus (RSV) (Yamamoto, et al., Cell, 22:787-797, (1980)); the herpes thymidine kinase promoter (Wagner et al., Proc. Natl Acad. Sci. U.S.A., 78:144-1445, (1981 )); the regulatory sequences of the metallothionine gene (Brinster et al., Nature, 296:39-42, (1982)); prokaryotic expression vectors such as the beta-lactamase promoter (Villa-Kamaroff, et al., Proc. Natl. Acad. Sci. U.S.A., 75:3727-3731, (1978)); or the tac promoter (DeBoer, et al., Proc. Natl. Acad. Sci. U.S.A., 80:21-25.(1983)). Also of interest are the following animal transcriptional control regions, which exhibit tissue specificity and have been utilized in transgenic animals: the elastase 1 gene control region which is active in pancreatic acmar cells (Swift et al., Cell, 38:639-646, (1984); Ornitz et al., Cold Spring Harbor Svmp Quant Biol. 50:399-409, (1986); MacDonald, Hepatology, 7:425-515, (1987)); the insulin gene control region which is active in pancreatic beta cells (Hanahan, Nature, 315:115-122, (1985)); the immunoglobulin gene control region which is active in lymphoid cells (Grosschedl et al., Cell, 38:647-658, (1984); Adames et al., Nature, 318:533-538, (1985); Alexander et al., Mol. Cell. Biol., 7:1436-1444, (1987)); the mouse mammary tumor virus control region which is active in testicular, breast, lymphoid and mast cells (Leder et al., Cell, 45:485-495, (1986)), the albumin gene control region which is active in liver (Pinkert et al , Genes and Devel., 1:268-276, (1987)); the alpha-feto-protein gene control region which is active in liver (Krumlauf et al., Mol. Cell. Biol., 5:1639-1648, 1985; Hammer et al., Science, 235:53-58, (1987)); the alpha 1-antitrypsin gene control region which is active in the liver (Kelsey et al., Genes and Devel., 1:161-171; (1987)); the beta-globin gene control region which is active in myeloid cells (Mogram et al., Nature, 315:338-340, (1985); Kollias et al., Cell, 46:89-94, (1986)); the myelin basic protein gene control region which is active in oligodendrocyte cells in the brain (Readhead et al , Cell, 48:703-712, (1987)); the myosin light chain-2 gene control region which is active in skeletal muscle (Sani, Nature, 314:283-286, (1985)); and the gonadotropic releasing hormone gene control region which is active in the hypothalamus (Mason et al., Science, 234:1372-1378, (1986)).

An enhancer sequence may be inserted into the vector to increase the transcription of a DNA encoding a ymkz5-receptor polypeptide of the present invention by higher eukaryotes. Enhancers are cis-acting elements of DNA, usually about 10-300 bp in length, that act on the promoter to increase its transcription. Enhancers are relatively orientation and position independent. They have been found 5′ and 3′ to the transcription unit. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation or upregulation of eukaryotic promoters. While an enhancer may be spliced into the vector at a position 5′ or 3′ to ymkz5-receptor DNA, it is typically located at a site 5′ from the promoter.

Expression vectors of the invention may be constructed from a starting vector such as a commercially available vector. Such vectors may or may not contain all of the desired flanking sequences. Where one or more of the desired flanking sequences set forth above are not already present in the vector, they may be individually obtained and ligated into the vector. Methods used for obtaining each of the flanking sequences are well known to one skilled in the art.

Preferred vectors for practicing this invention are those which are compatible with bacterial, insect, and mammalian host cells. Such vectors include, inter alia, pCRII, pCR3, and pcDNA3.1 (Invitrogen Company, Carlsbad. Calif.), pBSII (Stratagene Company, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII; Invitrogen), pDSR-alpha (PCT Publication No. WO 90/14364) and pFastBacDual (Gibco/BRL, Grand Island, N.Y.).

Additional suitable vectors include, but are not limited to, cosmids, plasmids, or modified viruses, but it will be appreciated that the vector system must be compatible with the selected host cell. Such vectors include, but are not limited to plasmids such as Bluescript® plasmid derivatives (a high copy number ColE1-based phagemid, Stratagene Cloning Systems Inc., La Jolla Calif.), PCR cloning plasmids designed for cloning Taq-amplified PCR products (e.g., TOPO™ TA Cloning® Kit, PCR2.® plasmid derivatives, Invitrogen, Carlsbad, Calif.), and mammalian , yeast or virus vectors such as a baculovirus expression system (pBacPAK plasmid derivatives, Clontech, Palo Alto, Calif.). The recombinant molecules can be introduced into host cells via transformation, transfection, infection, electroporation, or other known techniques.

After the vector has been constructed and a nucleic acid molecule encoding an ymkz5-receptor polypeptide has been inserted into the proper site of the vector, the completed vector may be inserted into a suitable host cell for amplification and/or polypeptide expression.

Host cells may be prokaryotic host cells (such as E coli) or eukaryotic host cells (such as a yeast cell, an insect cell, or a vertebrate cell). The host cell, when cultured under appropriate conditions, synthesizes an ymkz5-receptor polypeptide which can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity, such as glycosylation or phosphorylation, and ease of folding into a biologically active molecule

Yeast and mammalian cells are preferred hosts of the present invention. The use of such hosts provides substantial advantages in that they can also carry out post-translational peptide modifications including glycosylation. A number of recombinant DNA strategies exist which utilize strong promoter sequences and high copy number of plasmids which can be utilized for production of the desired proteins in these hosts.

Yeast recognize leader sequences on cloned mammalian gene products and secrete peptides bearing leader sequences (i.e., pre-peptides). Mammalian cells provide post-translational modifications to protein molecules including correct folding or glycosylation at correct sites.

Nammalian cells which may be useful as hosts include cells of fibroblast origin such as VERO or CHO-K1, and their derivatives. For a mammalian host, several possible vector systems are available for the expression of the desired ymkz5-receptor protein. A wide variety of transcriptional and translational regulatory sequences may be employed, depending upon the nature of the host. The transcriptional and translational regulatory signals may be derived from viral sources. Such as adenovirus, bovine papilloma virus, simian virus, or the like, where the regulatory signals are associated with a particular gene which has a high level of expression. Alternatively, promoters from mammalian expression products, such as actin, collagen, myosin, etc., may be employed. Transcriptional initiation regulatory signals may be selected which allow for repression or activation, so that expression of the genes can be modulated. Useful signals are regulatory signals which are temperature-sensitive so that by varying the temperature, expression can be repressed or initiated, or are subject to chemical regulation, e.g., metabolite.

As widely known, translation of eukaryotic mRNA is initiated at the codon which encodes the first methionine. For this reason, it is preferable to ensure that the linkage between a eukaryotic promoter and a DNA sequence which encodes the desired receptor molecule does not contain any intervening codons which are capable of encoding a methionine (i.e., AUG). The presence of such codons results either in the formation of a fusion protein (if the AUG codon is in the same reading frame as the desired receptor molecule encoding DNA sequence) or a frame-shift mutation (if the AUG codon is not in the same reading frame as the desired ymkz5-receptor protein encoding sequence).

The expression of the ymkz5-receptor proteins can also be accomplished in procaryotic cells. Preferred prokaryotic hosts include bacteria such as E coli, Bacillus. Streptomyces, Pseudomonas, Salmonella, Serratia, etc. The most preferred prokaryotic host is E. coli. Bacterial hosts of particular interest include E. coli K12 strain 294 (ATCC 31446), E. coli X1776 (ATCC 31537), E coli W3110 (F⁻, lambda⁻, prototrophic (ATCC 27325)), and other enterobacteria (such as Salmonella typhimurium or Serratia marcescens), and various Pseudomonas species. The prokaryotic host must be compatible with the replicon and control sequences in the expression plasmid.

To express the desired ymkz5-receptor protein in a prokaryotic cell (such as, for example, E. coli, B. subulis, Pseudomonas, Streptomyces, etc.), it is necessary to operably link the desired receptor molecule encoding sequence to a functional prokaryotic promoter. Such promoters may be either constitutive or, more preferably, regulatable (i.e., inducible or derepressible). Examples of constitutive promoters include the int promoter of bacteriophage λ, and the bla promoter of the β-lactamase gene of pBR322), etc. Examples of inducible prokaryotic promoters include the major right and left promoters , of bacteriophagye λ (P_{I} and P_{R}), the trp, recA, lacZ, lacI, gal, and tac promoters of E. coli, the α-amylase (Ulmanen et al., J. Bacteriol. 162:176-182 (1985)), the σ-28-specific promoters of B. subulis (Gilman et al , Gene 32:11-20 (1984)), the promoters of the bacteriophages of Bacillus (Gryczan, In: The Molecular Biology of the Bacilli, Academic Press, Inc., (New York 1982)), and Streptomyces promoters (Ward et al., Mol Gen. Genet. 203:468-478 (1986)). Prokaryotic promoters are reviewed by Glick, (J. Ind. Microbiol 1:277-282 (1987)); Cenatiempo, Biochimue 68:505-516(1986)); and Gottesman, Ann. Rev Genet. 18:415-442(1984))

Proper expression in a prokaryotic cell also requires the presence of a ribosome binding site upstream from the gene-encoding, sequence. Such ribosome binding sites are disclosed, for example, by Gold. L. et al (Ann Rev. Microbiol. 35:365-404 (1981))

The desired ymkz5-receptor polypeptide encoding sequence and all operably linked promoter may be introduced into a recipient prokaryotic or eukaryotic cell either as a non-replicating DNA (or RNA) molecule, which may either be linear or, more preferably, a closed covalent circular molecule. Since such molecules are incapable of autonomous replication, the expression of the desired receptor molecule may occur through the transient expression of the introduced sequence. Alternatively, permanent expression may occur through the integration of the introduced sequence into the host chromosome.

In one embodiment, a vector is employed which is capable of integrating the desired gene sequences into the host cell chromosome. Cells which have stably integrated the introduced DNA into their chromosomes can be selected by also introducing one or more markers which allow for selection of host cells which contain the expression vector. The marker may complement an auxotrophy in the host (such as leu21, or ura3, which are common yeast auxotrophic markers), biocide resistance, e.g., antibiotics, or heavy metals, such as copper, or the like. The selectable marker gene can either be directly linked to the DNA gene sequences to be expressed, or introduced into the same cell by co-transfection.

In a preferred embodiment, the introduced sequence will be incorporated into a plasmid or viral vector capable of autonomous replication in the recipient host. Any of a wide variety of vectors may be employed for this purpose. Factors of importance in selecting a particular plasmid or viral vector include, for e.g. the case with which recipient cells that contain the vector may be recognized and selected from those recipient cells which do not contain the vector; the number of copies of the vector which are desired in a particular host; and whether it is desirable to be able to “shuttle” the vector between host cells of different species.

Any of a series of yeast gene expression systems can also be utilized. Examples of such expression vectors include the yeast 2-micron circle, the expression plasmids YEP13, I YVP and YRP, etc., or their derivatives. Such plasmids are well known in the art (Botstein, et al., Miami Wntr. Symp. 19:265-274 (1982); Broach, In: The Molecular Biology of the Yeast Saccharomyces: Life Cycle and Inheritance, Cold Spring Harbor Laboratory, Cold Spring Harbor, N.Y., p. 445-470 (1981); Broach, Cell 28:203-204 (1982)).

For a mammalian host, several possible vector systems are available for expression. One class of vectors utilize DNA elements which provide autonomously replicating extra-chromosomal plasmids, derived from animal viruses such as bovine papilloma virus, polyoma virus, adenovirus, or SV40 virus. A second class of vectors relies upon the integration of the desired gene sequences into the host chromosome. Cells which have stably integrated the introduced DNA into their chromosomes may be selected by also introducing one or more markers which allow selection of host cells which contain the expression vector. The marker may provide for prototropy to an auxotrophic host, biocide resistance, e.g., antibiotics, or heavy metals, such as copper or the like. The selectable marker gene can either be directly linked to the DNA sequences to be expressed, or introduced into the same cell by co-transformation. Additional elements may also be needed for optimal synthesis of mRNA. These elements may include splice signals, as well as transcription promoters, enhancers, and termination signals. The cDNA expression vectors incorporating such elements include those described by Okayama, H., Mol. Cell. Biol 3:280 (1983), and others. Preferred eukaryotic vectors include PWLNEO, PSV2CAT, POG44, PXT1, pSG, pSVK3, pBPV, pMSG, pSVL (Pharmacia).

Preferred prokaryotic vectors include plasmids such as those capable of replication in E coli such as, for example, pBR322, ColE1, pSC101, pACYC 184, πVX, pQE70, pQE60, pQE9, pBG, pD10, Phage script, psix 174, pbmescript SK, pbsks, pNH8A, pNH1Ba, pNH18A, pNH46A (SL rare gone), ptrc99a, pKK223-3,pKK233-3, pDR540, pRIT5. Such plasmids are, for example, disclosed by Maniatis, T., et al. (In: Molecular Cloning, A Laboratory Manual, Cold Spring, Harbor Press, Cold Spring, Harbor, N.Y. (1982)). Bacillus plasmids include pC194, pC221, pT127, etc. Such plasmids are disclosed by Gryczan, T. (In: The Molecular Biology of the Bacilli, Academic Press, New York ( 1982), pp 307-329). Suitable Streptomyces plasmids include pISJ101 (Kendall et al., J Bacteriol. 169:4177-4183 (1987)), and Streptomyces bacteriophages such as φC31 (Chater et al., In: Sixth International Symposium on Actmomycetales Biology, Akademiai Kaido, Budapest, Hungary (1986), pp 45-541). Pseudomonas plasmids are reviewed by John et al. (Rev. Infect. Dis. 8:693-704 (1986)), and Izaki, (Jpn. J. Bacteriol,33:729-742 (1978)). However, any other plasmid or vector may be used as longs as they are replicable and viable in the host cell.

Once the vector or DNA sequence containing the constructs has been prepared for expression, the DNA constructs may be introduced into all appropriate host. Various techniques may be employed, such as a protoplast fusion, calcium phosphate precipitation, electroporation or other conventional techniques. After the fusion, the cells are grown in media and screened for appropriate activities. Expression of the sequence results in the production of the ymkz5-receptor protein.

Suitable host cells or cell lines may be mammalian cells, such as Chinese hamster ovary cells (CHO: ATCC no. CCL-61), human embryonic kidney (HEK; ATCC NO. CRL), 293 or 293T cells (ATCC No. CCRL-1573), 3T3 cells (ATCC No. CCL92), mouse neuroblastoma N2A cells (ATCC no. CCL-131), HeLa (ATCC No. CCL-2), mouse L-929 cells (ATCC NO. CCL-1), BHK (ATCC No. CCL-10) or HaK (ATCC No. CCL-15) hamster cell lines. The selection of suitable mammalian host cells and methods for transformation, culture, amplification, screening and product production and purification are known in the art. Other suitable mammalian cell lines, are the monkey COS-1 (ATCC No. CRL-1650) and COS-7 (ATCC No. CRL-1651)cell lines, and the CV-1 cell line (ATCC No. CCL-70()). Further exemplary mammalian host cells include primate cell lines and rodent cell lines, including, transformed cell lines. Normal diploid cells, cell strains derived from in vitro culture of primary tissue, as well as primary explants, are also suitable. Candidate cells may be genotypically deficient in the selection gene, or may contain a dominantly acting selection gene.

Similarly useful as host cells suitable for the present invention are bacterial cells. For example, the various strains of E. coli (e.g., HB101(ATCC No. 33694), DH50α DH10, and MC1061(ATCC No 53338)) are well-known as host cells in the field of biotechnology. Various strains of B. subulis, Pseudomonas spp., other Bacillus spp., Streptomyces spp., and the like may also be employed in this method.

Many strains of yeast cells known to those skilled in the art are also available as host cells for the expression of the polypeptides of the present invention (e g. Saccharomyces, Pichia, Candida, Hansenula, and Torulopsis). (Bitter, G. , Heterologous Gene Expression in Yeast in: Berger and Kimmel, 152:673-684, (1987)). Preferred yeast strains include, for example, Saccharomyces cerevisiae, which can be transformed readily with DNA either by preparation of spheroplasts or by treatment as with alkaline salts such as LiCl. (Itoh et al., J. Bacteriol 153:163 (1983)). Some proteins expressed in yeast cells are efficiently secreted into the culture medium while others accumulate intracellularly

Additionally, where desired, insect cell systems may be utilized in the methods of the present invention. Such systems are described for example in Kitts et al (Biotechniques,14:810-817 (1993)). Lucklow (Curr. Opin, Biotechnol., 4:564-572 (1993)) and Lucklow et al. (J. Virol., 67:4566-4579 (1993 )). Preferred insect cells are Sf-9 and H15 (Invitrogen, Carlsbad, Calif.). Baculovirus vectors based on the Autographs californianuclear polyhedrosis virus, which are useful for the introduction of genetic information into insect cells include, but are not limited to pVL1392 or 1393 (Invitrogen).

Transformation or transfection of an expression vector for a ymkz5-receptor polypeptide into a selected host cell may be accomplished by methods such as calcium chloride, electroporation, microinjection, lipofection or the DEAE-dextran method. The method selected will in part be a function of the type of host cell to be used. These methods and other suitable methods are well known to the skilled artisan, and are set forth, for example, in Sambrook et al , supra.

One may also use transgenic animals to express glycosylated ymkz5 like polypeptides. For example, one may use a transgenic milk-producing animal (a cow or goat, for example) and obtain the present glycosylated polypeptide in the animal milk. One may also use plants to produce ymkz5-receptor polypeptides, however, in general, the glycosylation occurring in plants is different from that produced in mammalian cells, and may result in a glycosylated product which is not suitable for human therapeutic use.

Polypeptide Production

Host cells comprising an ymkz5-receptor expression vector (i.e., transformed or transfected) may be cultured using standard media well known to the skilled artisan. The media will usually contain all nutrients to allow for the growth and survival of the cells. Suitable media for culturing E. coli cells are for example, Luria Broth (LB) and/or Terrific Broth (TB) Suitable media for culturing eukaryotic cells arc Rosewell Park Memorial Institute Medium 1640 (RPMI 1640), Minimal Essential Medium (MEM), Dulbecco's Modified Eagle Medium (DMEM), of which may be supplemented with serum and/or growth factors as required by the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of transfected or transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

The amount of ymkz5-receptor polypeptide produced by a host cell can be evaluated using standard methods known in the art. Such methods include, without limitation, Western blot analysis, SDS-polyacrylamide gel electrophoresis, non-denaturing gel electrophoresis, HPLC separation, immunoprecipitation, and/or activity assays.

If a ymkz5-receptor polypeptide has been designed to be secreted from the host cells, the majority of polypeptide may be found in the cell culture medium. If however, the ymkz5-receptor polypeptide is not secreted from the host cells, it will be present in the cytoplasm and/or nucleus (for eukaryotic host cells) or in the cytosol (for bacterial host cells).

For a ymkz5-receptor polypeptide situated in the host cell cytoplasm and/or nucleus (for eukaryotic host cells) or in the cytosol (for bacterial host cells), intracellular material (including inclusion bodies for gram-negative bacteria) can be extracted from the host cell using any standard technique known to the skilled artisan. For example, the host cells can be lysed to release the contents of the periplasm/cytoplasim by French press, homogenization, and/or sonication followed by centrifugation.

Purification of a ymkz5-receptor polypeptide from solution can be accomplished using a variety of techniques If the polypeptide has been synthesized such that it contains a tag such as Hexahistidine (ymkz5-receptor polypeptide/hexaHis) or other small peptide such as FLAG (Eastman Kodak Co., New Haven, Conn.) or myc (Invitrogen. Carlsbad, Calif.) or the IgG Fc fragment fused at either its carboxyl or amino terminus, it may essentially be purified in a one-step process by passing the solution through an affinity column where the column matrix has a high affinity for the tag or for the polypeptide directly (i.e , a monoclonal antibody specifically recognizing ymkz5-receptor polypeptide). For example, polyhistidine binds with great affinity and specificity to nickel, thus an affinity column of nickel (such as the Qiagen® nickel columns) can be used for purification of ymkz5-receptor polypeptide/polyHis. (See for example, Ausubel et al , eds., Current Protocols in Molecular Biology, Section 10.11.8, S. John Wiley & Sons, New York (1993))

Where a ymkz5-receptor polypeptide is prepared without a tag attached, and no antibodies are available, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity.

If a ymkz5-receptor polypeptide is produced intracellularly, the intracellular material (including inclusion bodies for gram-negative bacteria) can be extracted from the host cell using, any standard technique known to the skilled artisan. For example, the host cells can be lysed to release the contents of the periplasm/cytoplasm by French press, homogenization, and/or sonication followed by centrifugation.

If a ymkz5-receptor polypeptide has formed inclusion bodies in the cytosol, the inclusion bodies can often bind to the inner and/or outer cellular membranes and thus will be found primarily in the pellet material after centrifugation. The pellet material can then be treated at pH extremes or with chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The ymkz5-receptor polypeptide in its now soluble form can then be analyzed using gel electrophoresis, immunoprecipitation or the like If it is desired to isolate the ymkz5-receptor polypeptide, isolation may be accomplished using standard methods described herein and in Marston et al. (Meth. Enz., 182:264-275 (1990)).

In some cases, a ymkz5-receptor polypeptide may not be biologically, active upon isolation. Various methods for “refolding” or converting the polypeptide to its tertiary structure and generating disulfide linkages, can be used to restore biological activity Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same chaotropes as used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol(DTT)/dithiane DTT, 2-mercaptoethanol(βME)/dithio-β(ME). A cosolvent may be used to increase the efficiency of the refolding, and the more common reagents used for this purpose include glycerol, polyethylene glycol of various molecular weights, arginine and the like.

If inclusion bodies are not formed to a significant degree upon expression of a ymkz5-receptor polypeptide, then the polypeptide will be found primarily in the supernatant after centrifugation of the cell homogenate. The polypeptide may be further isolated from the supernatant using methods such as those described herein.

Additionally, the ymkz5-receptor polypeptide may be purified through the use of a monoclonal antibody which is capable of specifically recognizing and binding to the ymkz5-receptor polypeptide.

Suitable procedures for purification thus include, without limitation, affinity chromatography, immunoaffinity chromatography, ion exchange chromatography, molecular sieve chromatography, High Performance Liquid Chromatography (HPLC), electrophoresis (including native gel electrophoresis) followed by gel elution, and preparative isoelectric focusing (“Isoprime” machine/technique. Hoefer Scientific, San Francisco, Calif.). In some cases, two or more purification techniques may be combined to achieve increased purity.

Ymkz5-receptor polypeptides, fragments, and/or derivatives thereof may also be prepared by chemical synthesis methods (such as solid phase peptide synthesis) using techniques known in the art such as those set forth by Merrifield et al , (J Am Chem Soc., 85:2149 (1963)), Houghten et al. (Proc. Natl Acad. Sci. USA, S82:5132 (1985)), and Stewart and Young (Solid Phase Peptide Synthesis, Pierce Chemical Co., Rockford, Ill. (1984)). Such polypeptides may be synthesized with or without a methionine on the amino terminus. Chemically synthesized ymkz5-receptor polypeptides or fragments may be oxidized using methods set forth in these references to form disulfide bridges. Chemically synthesized ymkz5-receptor polypeptides, fragments or derivatives are expected to have comparable biological activity to the corresponding ymkz5-receptor polypeptides, fragments or derivatives produced recombinantly or purified from natural sources, and thus may be used interchangeably with recombinant or natural ymkz5-receptor polypeptide.

Another means of obtaining ymkz5-receptor polypeptide is via purification from biological samples such as source tissues and/or fluids in which the ymkz5-receptor polypeptide is naturally found. Such purification can be conducted using methods for protein purification as described herein. The presence of the ymkz5-receptor polypeptide during purification may be monitored using, for example, an antibody prepared against recombinantly produced ymkz5-receptor polypeptide or peptide fragments thereof.

Chemically synthesized ymkz5 like polypeptides may be oxidized using methods set forth in these references to form disulfide bridges. Chemically synthesized ymkz5-receptor polypeptides are expected to have comparable biological activity to the corresponding ymkz5-receptor polypeptides produced recombinantly or purified from natural sources, and thus may be used interchangeably with a recombinant or natural ymkz5-receptor polypeptide.

Another means of obtaining an ymkz/5-receptor polypeptide is via purification from biological samples such as source tissues and/or fluids in which the ymkz5-receptor polypeptide is naturally found. Such purification can be conducted using methods for protein purification as described herein. The presence of the ymkz5-receptor polypeptide during purification may be monitored using, for example, an antibody prepared against recombinantly produced ymkz5-receptor polypeptide or peptide fragments thereof.

A number of additional methods for producing nucleic acids and polypeptides are known in the art, and can be used to produce polypeptides having specificity for ymkz5-receptor. See for example, Roberts et al., Proc. Natl Acad. Sci U.S.A., 94:12297-12303 (1997), which describes the production of fusion proteins between an mRNA and its encoded peptide. See also Roberts, Curr. Opin Chem. Biol., 3:268-273 (1999). Additionally, U.S. Pat. No. 5,824,469 describes methods of obtaining oligonucleotides capable of carrying out a specific biological function. The procedure involves generating a heterogeneous pool oligonucleotides, each having a 5′ randomized sequence, a central preselected sequence, and a 3 randomized sequence. The resulting heterogeneous pool is introduced into a population of cells that do not exhibit the desired biological function. Subpopulations of the cells are then screened for those which exhibit a predetermined biological function. From that subpopulation, oligonucleotides capable of carrying out the desired biological function are isolated.

U.S. Pat. Nos. 5,763,192, 5,814,476, 5,723,323, and 5,817,483 describe processes for producing peptides or polypeptides. This is done by producing stochastic genes or fragments thereof, and then introducing these genes into host cells which produce one or more proteins encoded by the stochastic genes. The host cells are then screened to identify those clones producing peptides or polypeptides having the desired activity.

Another method for producing peptides or polypeptides is described in PCT/US98/20094 (WO99/15650) filed by Athersys, Inc. Known as “Random Activation of Gene Expression for Gene Discovery” (RAGE-GD), the process involves the activation of endogenous gene expression or over-expression of a gene by in situ recombination methods. For example, expression of an endogenous gene is activated or increased by integrating a regulatory sequence into the target cell which is capable of activating expression of the gene by non-homologous or illegitimate recombination. The target DNA is first subjected to radiation, and a genetic promoter inserted. The promoter eventually locates a break at the front of a gene, initiating transcription of the gene. This results in expression of the desired peptide or polypeptide.

It will be appreciated that these methods can also be used to create comprehensive IL-17 like protein expression libraries, which can subsequently be used for high throughput phenotype screening in a variety of assays, such as biochemical assays, cellular assays, and whole organism assays (e g., plant, mouse, etc.).

Proteins, Polypeptides, Fragments, Variants and Muteins of ymkz5:

Polypeptides of the invention include isolated ymkz5-receptor polypeptides and polypeptides related thereto including fragments, variants, fusion polypeptides, and derivatives as defined herein above.

Ymkz5-receptor fragments of the invention may result from truncations at the amino terminus (with or without a leader sequence), truncations at the carboxy terminus, and/or deletions internal to the polypeptide. Most deletions and insertions, and substitutions in particular, are not expected to produce radical changes in the characteristics of the ymkz5-receptor protein. However, when it is difficult to predict the exact effect of the substitution, deletion, or insertion in advance of doing so, one skilled in the art will appreciate that the effect will be evaluated by routine screening assays. For example, a variant typically is made by site-specific mutagenesis of the ymkz5-receptor polypeptide encoding nucleic acid, expression of the variant nucleic acid in recombinant cell culture, and, optionally, purification from the cell culture, for example, by immunoaffinity adsorption on a polyclonal anti ymkz5-receptor anitibody column (to absorb the variant by binding it to at least one remaining immune epitope). In preferred embodiments, truncations and/or deletions comprise about 10 amino acids, or about 20 amino acid, or about 50 amino acids, or about 75 amino acids, or about 100 amino acids, or more than about 100 amino acids. The polypeptide fragments so produced will comprise about 25 contiguous amino acids, or about 50 amino acids, or about 75 amino acids, or about 100 amino acids, or about 150 amino acids, or about 175 amino Such ymkz5-receptor polypeptides fragments may optionally comprise an amino terminal methionine residue.

Ymkz5-receptor polypeptide variants of the invention include one or more amino acid substitutions, additions and/or deletions as compared to SEQ ID NO: 8. In preferred embodiments, the variants have from 1 to 3, or from 1 to 5, or from 1 to 10, or from 1 to 15, or from 1 to 20, or from 1 to 25, or from 1 to 50, or from 1 to 75, or from 1 to 100, or more than 100 amino acid substitutions, insertions, additions and/or deletions, wherein the substitutions may be conservative, as defined above, or non-conservative or any combination thereof. More particularly ymkz5-receptor variants may comprise the amino acid sequence set out as SEQ ID NO:8, wherein one or more amino acids from the group conisisting of amino acids 2, 3, 4, 5, 6, 7, 9, 10, 11, 12, 13, 14, 15, 16, 17, 18, 19, 20, 21, 22, 23, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151,, 151, 152, 153, 154, 155, 156, 157, 158, 158, 160, 160, 161, 162, 163, 164, 165, 166, 167, 168, 169, 170, 171, 172, 173, 174, 175, and 176 is substituted with another amino acid. The variants may have additions of amino acid residues either at the carboxy terminus or at the amino terminus (with or without a leader sequence).

Preferred ymkz5-receptor polypeptide variants include glycosylation variants wherein the number and/or type of glycosylation sites has been altered compared to native ymkz5-receptor polypeptide. In one embodiment, ymkz5-receptor variants comprise a greater or a lesser number of N-linked glycosylation sites. An N-linked glycosylation site is characterized by the sequence: Asn-X-Ser or Thr, where the amino acid residue designated as X maybe any type of amino acid except proline. Substitution(s) of amino acid residues to create this sequence provides a potential new site for addition of an N-linked carbohydrate chain. Alteratively, substitutions to eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created.

One skilled in the art will be able to determine suitable variants of the native ymkz5-receptor polypeptide using well known techniques. For example, one may be able to predict suitable areas of the molecule that may be changed without destroying biological activity Also, one skilled in the art will realize that even areas that may be important for biological activity or for structure may be subject to conservative amino acid substitutions without destroying the biological activity or without adversely affecting the polypeptide structure.

For predicting suitable areas of the molecule that may be changed without destroying activity, one skilled in the art may target areas not believed to be important for activity. For example, when similar polypeptides with similar activities from the same species or from other species are known, one skilled in the art may compare the amino acid sequence of ymkz5-receptor polypeptide to such similar polypeptides. After making such a comparison, one skilled in the art would be able to determine residues and portions of the molecules that are conserved among similar polypeptides. One skilled in the art would know that changes in areas of the ymkz5-receptor molecule that are not conserved would be less likely to adversely affect biological activity and/or stricture. One skilled in the art would also know that, even in relatively conserved regions, one could have likely substituted chemically similar amino acids for the naturally occurring residues while retaining activity (e.g. conservative amino acid residue substitutions).

Also, one skilled in the art may review structure-function studies identifying residues in similar polypeptides that are important for activity or structure. In view of such a comparison, one skilled it the art can predict the importance of amino acid residues in ymkz5-receptor that correspond to amino acid residues that are important for activity or structure in similar polypeptides. One skilled in the art may opt for chemically similar amino acid substitutions for such predicted important amino acid residues of ymkz5-receptor.

If available, one skilled in the art can also analyze the crystal structure and amino acid sequence in relation to that structure in similar polypeptides. In view of that information, one skilled in the art may be able to predict the alignment of amino acid residues of ymkz5-receptor polypeptide with respect to its three dimensional structure. One skilled in the art may choose not to make radical changes to amino acid residues predicted to be on the surface of the protein, since such residues may be involved in important interactions with other molecules.

Moreover, one skilled in the all could generate test variants containing a single amino acid substitution at each amino acid residue. The variants could be screened using activity assays disclosed in this application. Such variants could be used to gather information about suitable variants. For example, if one discovered that a change to a particular amino acid residue resulted in destroyed activity, variants with such a change would be avoided. In other words, based on information gathered from such experiments, when attempting to find additional acceptable variants, one skilled in the art would have known the amino acids where further substitutions should be avoided either alone or in combination with other mutations.

Ymkz5-receptor fusion polypeptides of the invention comprise ymkz5-receptor polypeptides, fragments, variants, or derivatives fused to a heterologous peptide(s) or protein(s). Heterologous peptide(s) and protein(s) include, but are not limited to, an epitope to allow for detection and/or isolation of a ymkz5-receptor fusion polypeptide, a transmembrane receptor protein or a portion thereof, such as an extracellular domain, or a transmembrane, a ligand or a portion thereof which binds to a transmembrane receptor protein, an enzyme or portion thereof which is catalytically active, a protein or peptide which promotes oligomerization. Such as leucine zipper domain, and a protein or peptide which increase stability, such as an immunoglobulin constant region. A ymkz5-receptor polypeptide may be fused to itself or to a fragment, variant, or derivative thereof. Fusions may be made either at the amino terminus or at the carboxy terminus of a ymkz5-receptor polypeptide, and may be direct with no linker or adapter molecule or may be through a linker or adapter molecule, such as one or more amino acid residues up to about 20 amino acids residues, or up to about 50 amino acid residues. Alternatively, the ymkz5-receptor fusion protein may comprise one or two ymkz5-receptor polypeptides covalently linked to one or two TNF-receptor polypeptide(s), or a member of the TNF-receptor family or a cytokine receptor such as interleukin-1 R (IL-1 R) polypeptide. The receptors preferably are produced as fusion proteins using recombinant DNA technology. A linker or adapter molecule may also be designed with a cleavage site for a DNA restriction endonuclease or for proteolytic cleavage to allow for separation and subsequent folding of the fused moieties.

Also envisioned as a part of the invention are circularly permuted structural analogs of the ymkz5-receptor polypeptide.

The development of recombinant DNA methods has made it possible to study the effects of sequence transposition on protein folding, structure and function. The approach used in creating new sequences resembles that of naturally occurring pairs of proteins that are related by linear reorganization of their amino acid sequences (Cunningham, et al., Proc. Natl. Acad. Sci U.S.A. 76:3218-3222 (1979), Teahter & Erfle, J. Bacteriol. 172:3837-3841 (1990); Schimming et al., Eur. J Biochem 204.13-19 (1992); Yamiuchi and Minamikawa, FEBS Lett 260.127-130 (1991 ), MacGregor et al., FEBS Lett. 378:263-266, (1996)). The first in vitro application of this type of rearrangement to proteins was described by Goldenberg and Creighton (J Mol Biol 165:407-413, (1983)). A new N-terminus is selected at an internal site (breakpoint) of the original sequence, the new sequence having the same order of amino acids as the original from the breakpoint until it reaches an amino acid that is at or near the original C-terminus. At this point the new sequence is joined, either directly or through an additional portion of sequence (linker), to an amino acid that is at or near the original N-terminus, and the new sequence continues with the same sequence as the original until it reaches a point that is at or near the amino acid that was N-terminal to the breakpoint site of the original sequence, this residue forming the new C-terminus of the chain.

This approach has been applied to proteins which range in size from 58 to 462 amino acids (Goldenberg & Creighton, J. Mol. Biol. 165:407-413 (1983); Li & Coffino, Mol. Cell. Biol. 13:2377-2383 (1993)). The proteins examined have represented a broad range of structural classes, including proteins that contain predominantly a α-helix (interleukin-4; Kreitman et al., Cytokine 7:311-318 (1995)), β-sheet (interleukin-1; Horlick et al., Protein Eng. 5:427-431 (1992)), or mixtures of the two (yeast phosphoribosyl anthranilate isomerase; Luger et al , Science 243.206-210 (1989)).

In a preferred embodiment, a ymkz5-receptor polypeptide fragment, variant and/or derivative is fused to an Fc region of human IgG. In one example, a human IgG hinge, CH2 and CH3 region may be fused at either the N-terminus or C-terminus of the ymkz5-receptor polypeptides using methods known to the skilled artisan. In another example, a portion of a hinge regions and CH2 and CH3 regions may be fuse. The ymkz5-receptor Fc-fusion polypeptide so produce may be purified by use of a Protein A affinity Column (Pierce, Rockford, Ill.). In addition, peptide and proteins fused to an Fc region have been found to exhibit a substantially greater half-life in vivo than the unfused counterpart. Also, a fusion to an Fc region allows for dimerization/multimerization of the fusion polypeptide. The Fc region may be naturally occurring, Fc region, or may be altered to improve certain qualities such as therapeutic qualities, circulation time, reduce aggregation, etc.

Ymkz5-receptor polypeptide derivatives are also included in the scope of the present invention. Covalent modifications of the ymkz5-receptor proteins of the present invention are included within the scope of this invention. Variant ymkz5-receptor proteins may be conveniently prepared by in vitro synthesis. Such modifications may be introduced into the molecule by reacting targeted amino acid residues of the purified or crude protein with an organic derivatizing agent that is capable of reacting with selected side chains or terminal residues. The resulting covalent derivatives are useful in programs directed at identifying residues important for biological activity.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, orchloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or carboxylic acid anhydrides. Derivatization with these agents has the effect of reversing the charge of the lysinyl residues. Other suitable reagents for derivatizing α-amino-containing, residues include imidoesters such as methyl picolinimidate; pyridoxal phosphate; pyridoxal; chloroborohydride; trinitrobenzenesulfonic acid; O-methylissurea, 2,4 pentanedione; and transaminase catalyzed reaction with glyoxylate.

Arginyl residues are modified by reaction with one or several conventional reagents, among them phenylglyoxal, 2,3-butanedione, 1,2-cyclohexanedionic, and ninhydrin. Derivatization of arginine residues requires that the reaction be performed in alkaline conditions because of the high pKₐ of the guanidine functional group Furthermore, these reagents may react with the groups of lysine as well as the arginine Epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R¹) such as 1-cyclohexyl-3-(2-morpholinyl-(4-ethyl) carbodiimide or 1-ethyl-3 (4 azonia 4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking the “ymkz5-receptor proteins” to water-insoluble support matrixes or surfaces for use in the method for cleaving the ymkz5-receptor protein-fusion polypeptide to release and recover the cleaved polypeptide. Commonly used crosslinking agents include, e.g., 1,1-bis(diazoacetyl)-2-phenylethane, glutaraldehyde, N-hydroxysuccinimide esters, for example, esters with 4-azidosalicylic acid, homo-bifunctional imidoesters, including disuccinimidyl esters such as 3,3-dithiiobis(succinimidylpropioonate), and bifunctional maleimides such as bis-N-maleimido-1,8-octane. Derivatizing agents such as methyl-3-[p-azidophenyl) dithio]propioimidate yield photoactivatable intermediates that are capable of forming cross links in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440, incorporated herein by reference, are employed for protein immobilization.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Other modifications include hydroxylation of proline and lysine, phosphorylation of hydroxyl groups of seryl or theonyl residues, methylation of the α-amino groups of lysine, arginine, and histidine side chains (T. E. Creighton, Proteins Structure and Molecule Properties, W. H. Freeman & Co., San Francisco, pp. 79-86 (1983)), acetylation of the N-terminal amine, and, in some instances, amidation of the C-terminal carboxyl groups. Such derivatives are chemically modified ymkz5-receptor polypeptide compositions in which ymkz5-receptor polypeptide is linked to a polymer. The polymer selected is typically water soluble so that the protein to which it is attached does not precipitate in an aqueous environment, such as a physiological environment. The polymer selected is usually modified to have a single reactive group, such as an active ester for acylation or an aldehyde for alkylation, so that the degree of polymerization may be controlled as provided for in the present methods. The polymer may be of any molecular weight, and may be branched or unbranched. Included within the scope of the ymkz5-receptor polypeptide polymers is a mixture of polymers. Preferably, for therapeutic use of the end-product preparation, the polymer will be pharmaceutically acceptable.

The water soluble polymer or mixture thereof may be selected from the group consisting of, for example, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (e.g., glycerol) and polyvinyl alcohol.

For the acylation reactions, the polymer(s) selected should have a single reactive ester group. For reductive alkylation, the polymer(s) selected should have a single reactive aldehyde group. A preferred reactive aldehyde is polyethylene glycol propionaldehyde, which is water stable, or mono C1-C10 alkoxy or aryloxy derivatives thereof (see U.S. Pat. No. 5,252,714).

Pegylation of ymkz5-receptor polypeptides may be carried out by any of the pegylation reactions known in the art, as described for example in the following references Focus on Growth Factors 3: 4-10 (1992 ); EP 0 154 316; and EP 0 401 384 incorporated herein by reference. Preferably, the pegylation is carried out via an acylation reaction or an alkylation reaction with a reactive polyethylene glycol molecule (or an analogous reactive water-soluble polymer) as described below.

A particularly preferred water-soluble polymer for use herein is polyethylene glycol, abbreviated PEG. As used herein, polyethylene glycol is meant to encompass any of the forms of PEG that have been used to derivatize other proteins, such as mono-(C1-C10) alkoxy- or aryloxy-polyethylene glycol. PEG is a linear or branched neutral polyether, available in a broad range of molecular weights, and is soluble in water and most organic solvents. PEG is effective at excluding other polymers or peptides when present in water, primarily through its high dynamic chain mobility and hydrophibic nature, thus creating a water shell or hydration sphere when attached to other proteins or polymer surfaces. PEG is nontoxic, non-immunogenic, and approved by the Food and Drug Administration for internal consumption.

Proteins or enzymes when conjugated to PEG have demonstrated bioactivity, non-antigenic properties, and decreased clearance rates when administered in animals Veronese et al., Preparation and Properties of Monomethoxypoly(ethylene glyco.)—modified Enzymes for Therapeutic Applications, in J. M. Harris ed., Poly(Ethylene Clycol) Chemistry—Biotechnical and Biomedical Applications 127-36 (1992), incorporated herein by reference. This is due to the exclusion properties of PEG in preventing recognition by the immune system. In addition, PEG has been widely used in surface modification procedures to decrease protein adsorption and improve blood compatibility. Kim et al., Ann. N.Y. Acad. Sci. 516: 116-30) (1987); Jacobs et al., Artif Organs 12:500-01 (1988); Park et al., J Poly. Sci, Part A 29:1725-31 (1991), incorporated herein by reference. Hydrophobic polymer surfaces, such as polyurethanes and polystyrene were modified by the grafting, of PEG (MW 3,400) and employed as nonthrombogenic surfaces. In these studies, surface properties (contact angle) were more consistent with hydrophilic surfaces, due to the hydrating effect of PEG. More importantly, protein (albumin and other plasma proteins) adsorption was greatly reduced, resulting from the high chain motility, hydration sphere, and protein exclusion properties of PEG.

PEG (MW 3,4000) was determined as an optimal size in surface immobilization studies, K. D. Park et al., J. Biomed. Mat. Res. 26:739-45 (1992), while PEG (MW 5,000) was most beneficial in decreasing protein antigenicity. (F. M. Veronese et al., In Harris et al., Poly(Ethylene Glycol) Chemistry—Biotechnical and Biomedical Applications 127-36, supra., incorporated herein by reference)

In general, chemical derivatization may be performed under any suitable conditions used to react a biologically active substance with an activated polymer molecule. Methods for preparing pegylated ymkz5-receptor polypeptides will generally comprise the steps of (a) reacting the polypeptide with polyethylene glycol (such as a reactive ester or aldehyde derivative of PEG) under conditions whereby ymkz5-receptor polypeptide becomes attached to one or more PEG groups, and (b) obtaining the reaction product(s). In general, the optimal reaction conditions for the acylation reactions will be determined based on known parameters and the desired result. For example, the larger the ratio of PEG: protein, the greater the percentage of poly-pegylated product.

In a preferred embodiment, the ymkz5-receptor polypeptide derivative will have a single PEG moiety at the N terminus. See U.S. Pat. No. 8,234,784, herein incorporated by reference.

In another embodiment, ymkz5 like polypeptides may be chemically coupled to biotin, and the biotin/ymkz5-receptor polypeptide molecules which are conjugated are then allowed to bind to avidin, resulting in tetravalent avidin/biotin/ymkz5 like polypeptide molecules. Ymkz5-receptor polypeptides may also be covalently coupled to dinitrophenol (DNP) or trinitrophenol (TNP) and the resulting conjugates precipitated with anti-DNP or anti-TNP-IgM to form decameric conjugates with a valency of 10.

Generally, conditions which may be alleviated or modulated by administration of the present ymkz5-receptor polypeptide derivative include those described herein for ymkz5-receptor polypeptides. However, the ymkz5-receptor polypeptide derivative disclosed herein may have additional activities, enhanced or reduced biological activity, or other characteristics, such as increased or decreased half-life, as compared to the non-derivatized molecules.

Selective Binding Agents

As used herein, the term “selective binding agent” refers to a molecule which has specificity for one or more ymkz5-receptor polypeptides. Suitable selective binding agents include, but are not limited to, antibodies and derivatives thereof, polypeptides, anitsense oligonucleotides, and small molecules. Suitable selective binding agents may be prepared using methods known in the art. An exemplary ymkz5-receptor polypeptide selective binding agent of the present invention is capable of binding a certain portion of the ymkz5-receptor polypeptide thereby inhibiting the binding of the polypeptide to the ymkz5-receptor polypeptide receptor(s).

Selective binding agents such as antibodies and antibody fragments that bind ymkz5-receptor polypeptides are within the scope of the present invention. The antibodies may be polyclonal including monospecific polyclonal, monoclonal (MAbs), recombinant, chimeric, humanized such as CDR-grafted, human, single chain, and/or bispecific, as well as fragments, variants or derivatives thereof. Antibody fragments include those portions of the antibody which bind to an epitope on the ymkz5-receptor polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward a ymkz5-receptor polypeptide generally are produced in animals (e.g., rabbits or mice) by means of multiple subcutaneous or intraperitoneal injections of ymkz5-receptor and an adjuvant. It may be useful to conjugate a ymkz5-receptor polypeptide, or a variant, fragment or derivative thereof to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for anti-“ymkz5-receptor antibody” titer.

Monoclonal antibodies directed toward ymkz5-receptor polypeptides are produced using any method which provides for the production of anitibody molecules by continuous cell lines in culture. Examples of suitable methods for preparing monoclonal antibodies include the hybridoma methods of Kohler, et al., Nature 256: 495-497 (1975), and the human B-cell hybridoma method, Kozbor, J. Immunol., 133: 3001 (1984); Brodeur, et al., Monoclonal Antibody Production Techniques and Applications, pp. 51-63 (Marcel Dekker, Inc.. New York. 1987).

Also provided by the invention are hybridoma cell lines which produce monoclonal antibodies reactive with ymkz5-receptor polypeptides.

Monoclonal antibodies of the invention may be modified for use as therapeutics. One embodiment is a “chimeric” antibody in which a portion of the heavy and/or light chain is identical with or homologous to corresponding sequence in antibodies derived from a particular species or belonging to a particular antibody class or subclass, while the remainder of the chain(s) is identical with or homologous to corresponding sequence in antibodies derived from another species or belonging to another antibody class or subclass. As included are as fragments of such antibodies, so long, as they exhibit the desired biological activity (See U.S. Pat. No. 4,816,567; Morrison, et al., Proc. Natl. Acad. Sci. U.S.A.. 81, 6851-6855 (1985).

In another embodiment, a monoclonal antibody of the invention is a “humanized” antibody. Methods for humanizing non-human antibodies are well known in the art. Generally, a humanized antibody has one or more amino acid residues introduced into it from a source which is non-human. Humanization can be performed following methods known in the art (Jones et al., Nature 321: 522-525 (1986); Riechmann, et al., Nature 332: 323-327 (1988); Verhoeyen et al., Science 239: 1534-1536 (1988)), by substituting rodent complementarity-determining regions (CDRs) for the corresponding regions of a human anitibody.

Also encompassed by the invention are fully human antibodies which bind ymkz5-receptor polypeptides, fragments, variants and/or derivatives. Using transgenic animals (e.g., mice) that are capable of producing a repertoire of human antibodies in the absence of endogenous immunoglobulin production such antibodies are produced by immunization with a ymkz5-receptor antigen (i.e., having at least 6 contiguous amino acids), optionally conjugated to a carrier. See, for example, Jakobovits et al , Proc. Natl. Acad. Sci. U.S.A., 90: 2551-2555( 1993); Jakobovits et al., Nature 362: 255-258 (1993); Bruggermann et al., Year in Immuno. 7:33 (1993). In one method, such transgenic animals are produced by incapacitating the endogenous loci encoding the heavy and light immunoglobulin chains therein, and inserting loci encoding human heavy and light chain proteins into the genome thereof. Partially modified animals, that is those having less than the full complement of modifications, are then cross-bred to obtain an animal having all of the desired immune system modifications When administered an immunogen, these transgenic animals produce antibodies with human (rather than e.g., murine) amino acid sequences, including variable regions which are immunospecific for these antigens. See PCT Application Nos. PCT/US96/05928 and PCT/US93/06926. Additional methods are described in U.S. Pat. No. 5,545,807, PCT Application Nos. PCT/US91/245, PCT/GB89/01207, and in EP 546073B1 and EP 546073A1. Human antibodies may also be produced by the expression of recombinant DNA in host cells or by expression in hybridoma cells as described herein.

In an alternative embodiment, human antibodies can be produced from phage-display libraries (Hoogenboom et al., J. Mol. Biol. 227:381 (1991); Marks et al., J. Mol. Biol. 222:581 (1991). These processes mimic immune selection through the display of antibody repertoires on the surface of filamentous bacteriophage, and subsequent selection of phage by their binding to an antigen of choice. One such technique is described in PCT Application no. PCT/US98/17364, which describes the isolation of high affinity and functional agonistic antibodies for MPL- and msk-receptors using such an approach.

Chimeric, CDR grafted, and humanized antibodies are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herin.

The anti-ymkz5-receptor antibodies of the invention may be employed in any known assay method, such as competitive binding assays, direct and indirect sandwich assays, and immunoprecipitation assays (Sola, Monoclonal Antibodies: A Manual of Techniques, pp. 147-158 (CRC Press, Inc., 1987)) for the detection and quantitation of ymkz5-receptor polypeptides. The antibodies will bind ymkz5-receptor polypeptides with an affinity which is appropriate for the assay method being employed.

For diagnostic applications, anti-ymkz5-receptor antibodies typically will be labeled with a detectable moiety. The detectable moiety can be any one which is capable of producing, either directly or indirectly, a detectable signal For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, β-galactosidase or horseradish peroxidase. Bayer et al., Meth. Enz. 184: 138-163 (1990)).

The anti-ymkz5-receptor antibodies of the invention may be employed in any known assay method, such as competitive binding assays, direct and indirect sandwich assays, and immunoprecipitation assays (Sola, Monoclonal Antibodies: A Manual of Techniques, pp. 147-158 (CRC Press, Inc., 1987)) for the detection and quantitation of ymkz5-receptor polypeptides. The antibodies will bind ymkz5-receptor polypeptides with an affinity which is appropriate for the assay method being employed.

The activity of the cell lysate or purified ymkz5-receptor protein variant is then screened in a suitable screening assay for the desired characteristic. For example, a change in the binding affinity for a ligand or immunological character of the ymkz5 receptor protein, such as affinity for a given antibody, is measured by a competitive type immunoassay. Changes in immunomodulation activity are measured by the appropriate assay. Modifications of such protein properties as redox or thermal stability hydrophobicity, susceptibility to proteolytic degradation or the tendency to aggregate with carriers or into multimers are assayed by methods well known to the ordinarily skilled artisan. Competitive bindings assays rely on the ability of a labeled standard (e.g., a ymkz5-receptor polypeptide, or an immunologically reactive portion thereof) to compete with the test sample analyte (a ymkz5-receptor polypeptide) for binding with a limited amount of antibody. The amount of a ymkz5-receptor polypeptide in the test sample is inversely proportional to the amount of standard that becomes bound to the antibodies. To facilitate determining the amount of standard that becomes bound, the antibodies typically are insolubilized before or after the competition, so that the standard and analyte that are bound to the antibodies may conveniently be separated from the standard and analyte which remain unbound.

Sandwich immuno-assays typically involve the use of two antibodies, each capable of binding to a different immunogenic portion, or epitope, of the protein to be detected and/or quantitated. In a sandwich assay, the test sample analyte is typically bound by a first antibody which is immobilized on a solid support, and thereafter a second antibody binds to the analyte, thus forming an insoluble three part complex. See e g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assays). For example, one type of sandwich assay is an enzyme-linked immunosorbant assay (ELISA), in which case the detectable moiety is an enzyme.

The anti-ymkz5-receptor antibodies of the invention also are useful for in vivo imaging. An antibody labeled with a detectable moiety is administered to an animal, preferably into the bloodstream, and the presence and location of the labeled antibody in the host is assayed. The antibody may be labeled with any moiety that is detectable in an animal, whether by nuclear magnetic resonance, radiology, or other detection means known in the art.

Selective binding agent of the invention including anti-ymkz5 receptor antibodies may be used as therapeutics. Therapeutic antibodies are generally agonists or antagonists, in that they either enhance or reduce, respectively, at least one of the biological activities of a ymkz5-receptor polypeptide. In one embodiment, antagonist antibodies of the invention are antibodies or binding fragments thereof which are capable of specifically binding to a ymkz5-receptor polypeptide, fragment, variant and/or derivative, and which are capable of inhibiting or eliminating the functional activity of a ymkz5-receptor polypeptide in vivo or in vitro. In preferred embodiments, an antagonist anitibody will inhibit the functional activity of a ymkz5-receptor polypeptide at least about 50%, preferably at least about 80%, more preferably 90%, and most preferably 100%. Agonist and antagonist anti-ymkz5-receptor antibodies are identified by screening assays described herein.

Ymkz5-receptor polypeptides can be used to clone ymkz5-receptor ligand(s) using an “expression cloning” strategy. Radiolabeled (125-Iodine) ymkz5-receptor polypeptide or “affinity/activity-tagged” ymkz5-receptor polypeptide such as an Fc fusion or an alkaline phosphatase fusion) can be used in binding assays to identify a cell type or a cell line or tissue that expresses ymkz5-receptor ligand(s). RNA isolated from such cells or tissues can then be converted to cDNA, cloned into a mammalian expression vector, and transfected into mammalian cells (for example, COS, or 293) to create an expression library. Radiolabeled or tagged ymkz5-receptor polypeptide can then be used as all affinity reagent to identify and isolate the subset of cells in this library expressing ymkz5-receptor ligand(s). DNA is then isolated from these cells and transfected into mammalian cells to create a secondary expression library in which the fraction of cells expressing ymkz5-receptor ligand(s) would be many-fold higher than in the original library. This enrichment process can be repeated iteratively until a single recombinant clone containing, an ymkz5-receptor ligand is isolated. Isolation of ymkz5-receptor ligand(s) is useful for identifying or developing novel agonists and antagonists of the ymkz5-receptor signaling, pathway. Such agonists and antagonists include ymkz5-receptor ligand(s), anti-ymkz5-receptor ligand antibodies, small molecules or anitsense oligonucleotides.

Diagnostic Kits and Reagents

This invention also contemplates use of ymkz5-receptor proteins, fragments thereof, peptides, binding compositions, and their fusion products in a variety of diagnostic kits and methods for detecting the presence of receptors and/or antibodies, or ligands. Typically the kit will have a compartment containing a ymkz5 receptor peptide or gene segment or a reagent which recognizes one or the other, e.g., binding reagents.

A kit for determining the binding affinity of a ligand or test compound to the ymkz5-receptor would typically comprise a test compound; a labeled compound, for example an antibody having known binding affinity for the protein; or a source of ligand (naturally occuring or recombinant), and a means for separating bound from free labeled compound, such as a solid phase for immobilizing the ligand or receptor Once compounds are screened, those having suitable binding affinity to the ligand or receptor can be evaluated in suitable biological assays, as are well known in the art, to determine whether they act as agonists or antagonists to the receptor. The availability of recombinant chemokine or receptor polypeptides also provide well defined standards for calibrating such assays or as positive control samples.

A preferred kit for determining the concentration of, for example, ymkz5-receptor or ligand in a sample would typically comprise a labeled compound, e.g , antibody, having known binding affinity for the target, a source of ligand or receptor (naturally occurring or recombinant), and a means for separating, the bound from free labeled compound, for example, a solid phase for immobilizing the ligand or receptor. Compartments containing reagents, and Instructions for use or disposal, will normally be provided.

Antibodies, including antigen binding fragments, specific for the ligand or receptor, or fragments are useful in diagnostic applications to detect the presence of elevated levels of ligand, receptor, and/or its fragments. Such diagnostic assays can employ lysates, live cells, fixed cells, immunofluorescence, cell cultures, body fluids, and further can involve the detection of antigens related to the ligand or receptor in serum, or the like. Diagnostic assays may be homogeneous (without a separation step between free reagent and antigen complex) or heterogeneous (with a separation step). Various commercial assays exist, such as radioimmunoassay (RIA), enzyme-linked immunosorbant assay (ELISA), enzyme immunoassay (EIA), enzyme-multiplied immunoassay technique (EMIT), substrate-labeled fluorescent immunoassay (SLFIA), and the like. For example. unlabeled antibodies can be employed by using, a second antibody which is labeled and which recognizes the primary antibody to a ligand or receptor or to a particular fragment thereof. Similar assays have also been extensively discussed in the literature. (See, e.g., Harlow and Lane Antibodies: A Laboratory Manual, CSH (1988).)

Anti-idiotypic antibodies may have similar uses to diagnose presence of antibodies against a chemokine or receptor, as such may be diagnostic of various abnormal states. For example, overproduction of a chemokine or receptor may result in production of various immunological reactions which may be diagnostic of abnormal physiological states, particularly in various inflammatory or asthma conditions.

Frequently, the reagents for diagnostic assays are supplied in kits, so as to optimize the sensitivity of the assay. For the subject invention, depending upon the nature of the assay, the protocol, and the label, either labeled or unlabeled antibody or labeled chemokine or receptor is provided. This Is usually in conjunction with other additives, such as buffers, stabilizers, materials necessary for signal production such as substrates for enzymes, and the like. Preferably, the kit will also contain instructions for proper use and disposal of the contents after use. Typically the kit has compartments or containers for each useful reagent. Desirably, the reagents are provided as a dry lyophilized powder, where the reagents may be reconstituted in an aqueous medium providing appropriate concentrations of reagents for performing the assay.

The aforementioned constituents of the drub screening and the diagnostic assays may be used without modification or may ,be modified in a variety of ways. For example, labeling may be achieved by covalently or non-covalently joining a moiety which directly or indirectly provides a detectable signal. In any of these assays, the ligand, test compound, receptor, or antibodies thereto can be labeled either directly or indirectly. Possibilities for direct labeling include label groups: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring=, the chance in fluorescence intensity, wavelength shift, or fluorescence polarization. Possibilities for indirect labeling include biotinylation of one constituent followed by binding to avidin coupled to one of the above label groups.

There are also numerous methods of separating bound from the free ligand, or alternatively bound from free test compound. The chemokine or receptor can be immobilized on various matrixes, perhaps with detergents or associated lipids, followed by washing. Suitable matrixes include plastic such as an ELISA plate, filters, and beads. Methods of immobilizing, the chemokine or receptor to a matrix include, without limitation, direct adhesion to plastic, use of a capture antibody, chemical coupling, and biotin-avidin. The last step in this approach may involve the precipitation of anitgen/anitibody complex by any of several methods including those utilizing, e.g., an organic solvent such as polyethylene glycol or a salt such as ammonium sulfate. Other suitable separation techniques include, without limitation, the fluorescein antibody magnetizable particle method described in Rattle et al. (Clin. Chem .30:1457-1461(1984)), and the double antibody magnetic particle separation as described in U.S. Pat. No. 4,659,6178, incorporated herein by reference.

Methods for linking proteins or their fragments to the various labels have been extensively reported in the literature and do not require detailed discussion here. Many of the techniques involve the use of activated carboxyl groups either through the use of carbodiimide or active esters to form peptide bonds, the formation of thioethers by reaction of a mercapto group with an activated halogen such as chloroacetyl, or an activated olefin such as maleimide, for linkage, or the like. Fusion proteins will also find use in these applications.

Nucleic acid molecules of the invention may be used to map the locations of the ymkz5-receptor gene and related genes on chromosomes. Mapping may be done by techniques known in the art, such as PCR amplification. I situ hybridization, and FISH.

This invention is also related to the use of the ymkz5-receptor gene as part of a diagnostic assay for detecting diseases or susceptibility to diseases related to the presence of mutated ymkz5-receptor gene. Such diseases arc related to an abnormal expression of ymkz5-receptor, for example, abnormal cellular proliferation such as tumors and cancers.

Individuals carrying mutations in the human ymkz5-receptor gene may be detected at the DNA level by a variety of techniques. Nucleic acids for diagnosis may be obtained from a patient's cells, such as from blood, urine saliva tissue biopsy and autopsy material. The genomic DNA may be used directly for detection or may be amplified enzymatically by using PCR (Saiki et al., Nature 324:163-106 (198)S6)) prior to analysis. RNA or cDNA may also be used for the same purpose. As an example. PCR primers complementary to the nucleic acid encoding ymkz5-receptor polypeptide can be used to identify and analyze ymkz5 receptor mutations. For example, deletions and insertions can detected by a change in size of the amplified product in comparison to the normal zenotype. Point mutations can be identified by hybridizing amplified DNA to radiolabeled ymkz5 receptor RNA or alternatively radiolabeled ymkz5 receptor anitsense DNA sequences. Perfectly matched sequences can be distinguished from mismatched duplexes by RNase A digestion or by differences in melting temperatures

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing, formamide gradient gels in which the nobilities of different DNA fragments are retarded in the gel at different positions according to their specific melting or partial melting temperatures (see, e.g., Myers et al., Science, 230 1242(1985))

Sequence changes at specific locations may also be revealed by nuclease protection assays, such as RNase and S1 protection or the chemical cleavage method (e g , Cotton et al., PNAS, USA, 85:4397-4401 (1985)).

Thus, the detection of a specific DNA sequence may be achieved by methods such as hybridization, RNase protection, chemical cleavage, direct DNA sequencing or the use of restriction enzymes, (e.g., Restriction Fragment Length Polymorphisms (RFLP)) and Southern blotting of genomic DNA.

In addition to more conventional gel-electrophoresis and DA sequencing, mutations can also be detected by in situ analysis.

The present invention also relates to a diagnostic assay for detecting altered levels of ymkz5-receptor protein in various tissues since an over-expression of the proteins compared to normal control tissue samples may detect the presence of a disease or susceptibility to a disease, for example, tumors, cerebral malaria and hereditary periodic fever syndromes. Assays used to detect levels of ymkz5-receptor protein in a sample derived from a host are well-known to those of skill in the art and include radioimmunoassays, competitive-binding assays, Western Blot analysis, ELISA assays and “sandwich ” assay. An ELISA assay (Coligan, et al., Current Protocols in Immunology, 1(2), Chapter 6, (1991 )) partially comprises preparing an antibody specific to the ymkz5 receptor antigen, preferably a monoclonal antibody. In addition a reporter antibody is prepared against the monoclonal antibody. To the reporter antibody is attached a detectable reagent such as radioactivity, fluorescence or in this example a horseradish peroxidase enzyme. A sample is now removed from a host and incubated on a solid support, e g., a polystyrene dish, that binds the proteins in the sample. Any free protein binding sites on the dish are then covered by incubating with a non-specific protein like BSA. Next, the monoclonal antibody is incubated in the dish during which time the monoclonal antibodies attach to any ymkz5-receptor polypeptides attached to the polystyrene dish. All unbound monoclonal antibody is washed out with buffer. The reporter antibody linked to horseradish peroxidase is now placed in the dish resulting in binding of the reporter antibody to any monoclonal antibody bound to ymkz5-receptor. Unattached reporter antibody is then washed out. Peroxidase substrates are then added to the dish and the amount of color de eloped in a given time period is a measurement of the amount of ymkz5-receptor protein present in a given volume of patient sample when compared against a standard curve.

A competition assay may be employed wherein antibodies specific to ymkz5 receptor are attached to a solid support and labeled ymkz5-receptor and a sample derived from the host are passed over the solid support and the amount of label detected, for example, by liquid scintillation chromatography, can be correlated to a quantity, of ymkz5 receptor in the sample. In addition, a sandwich “immuno-assay as described above may also be carried out to quantify the amount of ymkz5-receptor polypeptide in a biological sample.

The sequences of the present invention are also valuable for chromosone identification and mapping. The sequence can be specifically targeted to and can hybridize with a particular location on an individual human chromosome. Moreover, there is a current need for identifying particular sites on the chromosome wherein a gene can be localized. Few chromosome marking reagents based on actual sequence data (repeat polymorphisms) are presently available for marking, chromosomal location. The mapping of DNAs to chromosomes according to the present invention is an important first step in correlating those sequences within genes associated with disease.

Briefly, sequences can be mapped to chromosomes by preparing PCR primers (preferably 15-25 bp) from the cDNA. Computer analysis of the 3′-untranslated region of the sequence is used to rapidly select primers that do not span more than one exon in the genomic DNA, thus complicating the amplification process. These primers then used for PCR screening of somatic cell hybrids containing individual human chromosomes. Only those hybrids containing the human gene corresponding, to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map ymkz5-receptor to its chromosome include in situ hybridization, prescreening with labeled flow-sorted chromosomes and preselection by hybridization to construct chromosome specific-cDNA libraries.

Fluorescence in situ hybridization (FISH) of cDNA clone to a metaphase chromosomal spread can be used to provide a precise chromosomal location in one step. This technique can be used with cDNA as short as 500 or 600 bases; however, clones larger than 2,000 bp have a higher likelihood of binding to a unique chromosomal location with sufficient signal intensity for simple detection. FISH requires use of genomic clones or clones from which the express sequence tag (EST) was derived, and the longer the better. For example, 2,000 bp is good. 4,000 is better, and more than 4,000 is probably not necessary to get good results a reasonable percentage of the time. For a review of this technique see Verma et al., Human Chromosomes: A Manual of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be on of between 50 and 500 potential causative genes. (This assumes 1 megabase mapping resolution and one gene per 20 kb).

The nucleic acid molecule(s) of the present invention are also used as anti-sense inhibitors of ymkz5-receptor expression. Such inhibition may be effected by nucleic acid molecules which are complementary to and hybridize to expression control sequences (triple helix formation) or to ymkz5-receptor mRNA. Anti-sense probes may be designed by available techniques using the sequence of ymkz5-receptor disclosed herein. Anti-sense inhibitors provide information relating to the decrease or absence of a ymkz5-receptor polypeptide in a cell or organism. The nucleic acid molecules of the invention may be used for gene therapy. Nucleic acid molecules which express ymkz5-receptor in vivo provide information relating to the effects of the polypeptide in cells or organisms. Ymkz5 receptor nucleic acid molecules, fragments and/or derivatives that do not themselves encode biologically active polypeptides may be useful as hybridization probes in diagnostic assays to test, either qualitatively or quantitatively, for the presence of ymkz5-receptor DNA or corresponding RNA in mammalian tissue or bodily fluid samples.

Ymkz5-receptor polypeptide fragments, variants, and/or derivatives, whether biologically active or not, are useful for preparing antibodies that bind to an ymkz5 receptor polypeptide. The antibodies may be used for in vivo and in vitro diagnostic purposes, such as in labeled form to detect the presence of ymkz5-receptor polypeptide in a body fluid or cell sample. The antibodies may bind to an ymkz5-receptor polypeptide so as to diminish or block at least one activity characteristic of an ymkz5-receptor polypeptide, or may bind to a polypeptide to increase an activity.

Genetically Engineered Non-Human Mammals

The present invention further includes non-human mammals such as mice, rats, rabbits, goats, or sheep in which the gene (or genes) encoding, ymkz5-receptor polypeptides in which either the native form of the gene(s) for that mammal or a heterologous ymkz5 -receptor polypeptide gene(s) is (are) over expressed by the mammal, thereby creating a “transgenic” mammal. Such transgenic mammals may be prepared using well known methods such as those described in U.S. Pat. No. 5,489,743 and PCT Publication No. WO94/28122, incorporated herein by reference.

Additionally included within the scope of the present invention are non-human mammals such as mice, rats, rabbits, goats, or sheep in which the gene (or genes) encoding a native ymkz5-receptor polypeptide has (have) been disrupted (“knocked out”) such that the level of expression of this gene or genes is (are) significantly decreased or completely abolished. Such mammals may be prepared using techniques and methods such as those described in U.S. Pat. No. 5,557,032, incorporated herein by reference

The present invention further includes non-human mammals in which the promoter for one or more of the ymkz5-receptor polypeptides of the present invention is either activated or inactivated (using homologous recombination methods as described below) to alter the level of expression of one or more of the native ymkz5-receptor polypeptides.

These non-human mammals may be used for drug candidate screening. The impact of a drug candidate on the mammal may be measured. For example, drug candidates may decrease or increase expression of the ymkz5-receptor polypeptide gene. In certain embodiments, the amount of ymkz5-receptor polypeptide or a fragment(s) that is produced may be measured after exposure of the mammal to the drug candidate. Additionally, certain embodiments, one may detect the actual impact of the drug candidate on the animal. For example, over expression of a particular gene may result, in, or be associated with, a disease or pathological condition. In such cases, one may test a drug candidate's ability to decrease expression of the gene or its ability to prevent or inhibit a pathological condition. In other examples production of a particular metabolic product such as a fragment of a polypeptide, may result in, or be associated with, a disease or pathological condition. In such cases, one may test a drug candidate's ability to decrease production of such a metabolic product or its ability to present or inhibit a pathological condition.

Microarray

It will be appreciated that DNA microarray technology can be utilized in accordance with the present invention. DNA microarrays are miniature, high density arrays of nucleic acids positioned on a solid support, such as glass. Each cell or element within the array has numerous copies of a single species of DNA which acts as a target for hybridization for its cognate mRNA. In expression profiling using DNA microarray technology, mRNA is first extracted from a cell or tissue sample and then converted enzymatically to fluorescently labeled cDNA. This maternal is hybridized to the microarray and unbound cDNA is removed by washing. The expression of discrete genes represented on the array is then visualized by quantitating the amount of labeled cDNA which is specifically bound to each target DNA. In this way, the expression of thousands of genes can be quantitated in a high throughput, parallel manner from a single sample of biological material.

This high throughput expression profiling has a broad range of applications with respect to the ymkz5 -receptor molecules of the invention, including, but not limited to: the identification and validation of ymkz5-receptor disease-related genes as targets for therapeutics; molecular toxicology of ymkz5-receptor molecules and inhibitors thereof; stratification of populations and generation of surrogate markers for clinical trials; and enhancing ymkz5-receptor-related small molecule drug discovered by aiding in the identification of selective compounds in high throughput screens (HTS).

Assays for Other Modulators of ymkz5-Receptor Polypeptide Activity:

In some situations, it may be desirable to identify molecules that are modulators, i.e., agonists or antagonists, of the activity of ymkz5 receptor polpeptide. Natural or synthetic molecules that modulate ymkz5 receptor can be identified using one or more of the screening assays described below. Such molecules may be administered either in an ex vivo manner, or in an in vivo manner by local or iv injection, or by oral delivery, implantation device, or the like.

The following definition is used herein for describing the assays:

“Test molecule(s)” refers to the molecule(s) that is/are under evaluation for the ability to modulate (i.e., increase or decrease) the activity of an ymkz5-receptor polypeptide. Most commonly, a test molecule will interact directly with an ymkz5-receptor polypeptide. However, it is also contemplated that a test molecule may also modulate ymkz5-receptor polypeptide activity indirectly, such as by affecting ymkz5-receptor gene expression, or by binding to an ymkz5-receptor ligand. In one embodiment, a test molecule will bind to a ymkz5-receptor polypeptide with an affinity constant of at least about 10⁻⁶M, preferably about 10⁻⁸M, more preferably about 10⁻⁹ M, and even more preferably about 10⁻¹⁰M.

Methods for identifying compounds which interact with ymkz5-receptor polypeptides are encompassed by the invention. In certain embodiments, a ymkz5-receptor polypeptide is incubated with a test molecule under conditions which permit the interaction of the test molecule to ymkz5-receptor polypeptide, and the extent of the interaction can be measured. The test molecules can be screened in a substantially purified form or in a crude mixture.

Test molecules may be nucleic acid molecules, proteins, peptides, carbohydrates, lipids or small molecular weight organic or inorganic molecule which interacts with ymkz5-receptor polypeptide to regulate its activity. Molecules which regulate ymkz5-receptor polypeptide expression include nucleic acids which are complementary to nucleic acids encoding an ymkz5-receptor polypeptide, or are complementary to nucleic acids sequences which direct or control the expression of ymkz5-receptor polypeptide, and which act as anti-sense regulators of expression.

Once a set of test molecules has been identified as interacting with ymkz5-receptor polypeptide, the molecules may be further evaluated for their ability to increase or decrease ymkz5-receptor activity. The measurement of the interaction of test molecules with ymkz5-receptor polypeptides may be carried out in several formats, including cell-based binding assays, membrane binding assays, solution-phase assays and immunoassays. In general, test molecules are incubated with a ymkz5-receptor polypeptide for a specified period of time and the extent of binding to a ymkz5-receptor polypeptide is determined by filtration, electrochemiluminescent (ECL, ORIGEN system by IGEN), cell-based or immunoassays.

Homogeneous assay technologies for radioactivity (SPA; Amersham) and time resolved fluorescence (HTRF, Packard) can also be implemented. Binding, can be detected by labeling with radioactive isotopes (¹²⁵I, ³⁵S, ³H), fluorescent dyes (fluorescein), lanthanides such as Europium (Eu³) chelates or cryptates, orbipyridyl-ruthenium (Ru²) complexes. It is understood that the choice of a labeled probe will depend upon the detection system used. Alternatively, a ymkz5-receptor polypeptide may be modified with an unlabeled epitope tag (e.g., biotin, peptides, His6, myc, Fc) and bound to proteins such as streptavidin, anti-peptide or anti-protein antibodies which have a detectable label as described above.

The interaction of test molecules to ymkz5-receptor polypeptides may also be assayed directly using polyclonal or monoclonal antibodies in an immunoassay. Alternatively, modified forms of ymkz5-receptor polypeptides containing eiptope tags as described herein may be used in solution and immunoassays.

In one embodiment, a ymkz5-receptor agonist or antagonist may be a protein, peptide, carbohydrate, lipid or small molecular weight molecule which interacts with ymkz5-receptor to regulate its activity. Potential protein antagonists of ymkz5-receptor include antibodies which bind to active regions of the polypeptide and inhibit or eliminate at least once activity of ymkz5-receptor. Molecules which regulate ymkz5-receptor polypeptide expression may include nucleic acids which are complementary to nucleic acids encoding a ymkz5-receptor polypeptide, or are complementary to nucleic acids sequences which direct or control expression of polypeptide, and which act as anti-sense regulators of expression.

In the event that ymkz5-receptor polypeptides display biological activity through an interaction with a ligand, a variety of in vitro assays may be used to measure binding of a ymkz5-receptor polypeptide to the corresponding binding partner (such as a selective binding agent or ligand). These assays may be used to screen test molecules for their ability to increase or decrease the rate and/or the extent of binding of a ymkz5-receptor polypeptide to its binding partner. In one assay, a ymkz5-receptor polypeptide is immobilized in the bottom of the wells of a microtiter plate. Radiolabeled ymkz5-receptor binding partner (for example, iodinated ymkz5-receptor binding partner) and the test molecule(s) can then be added either one at a time (in either order) or simultaneously to the wells. After incubation, the wells can be washed and counted, using, a scintillation counter for, radioactivity to determine the extent to which the binding partner bound to ymkz5-receptor polypeptide. Typically, the molecules will be tested over a range of concentrations, and a series of control wells lacking one or more elements of the test assays can be used for accuracy in the evaluation of the results. An alternative to this method involves reversing the “positions” of the proteins, i e., immobilizing ymkz5-receptor binding partner to the microtiter plate wells, incubating with the test molecule and radiolabeled ymkz5-receptor and determining the extent of ymkz5-receptor binding (see, for example, Chapter 18 of Current Protocols in Molecular Biology, Ausubel et al., eds., John Wiley &, Sons, New York, N.Y. ( 1995)).

As an alternative to radiolabelling, an ymkz5-receptor polypeptide or its binding partner may be conjugated to biotin and the presence of biotinylated protein can then be detected using streptavidin linked to an enzyme, such as horseradish peroxidase (HRP) or alkaline phosphatase (AP), that can be detected colormetrically, or by fluorescent tagging of streptavidin. An antibody directed to an ymkz5-receptor-polypeptide or to an ymkz5-receptor binding partner and is conjugated to biotin may also be used and can be detected after incubation with enzyme-linked streptavidin linked to AP or HRP.

A ymkz5-receptor polypeptide and a ymkz5-receptor binding partner can also be immobilized by attachment to agarose beads, acrylic beads or other types of such solid phase substrates. The substrate-protein complex can be placed in a solution containing the complementary protein and the test compound; after incubation, the beads can be precipitated by centrifugation, and the amount of binding between an ymkz5-receptor polypeptide and its binding partner can be assessed using the methods described herein. Alternatively, the substrate-protein complex can be immobilized in a column and the test molecule and complementary protein are passed through the column. The formation of a complex between an ymkz5-receptor polypeptide and its binding partner can then be assessed using any of the techniques set forth above, i.e., radiolabelling, antibody binding, or the like.

Another in vitro assay that is useful for identifying a test molecule which increase or decrease the formation of a complex between a ymkz5-receptor binding protein and a ymkz5-receptor binding partner is a surface plasmon resonance detector system such as the BIAcore assay system (Pharmacia, Piscataway, N.J.). The BIAcore system may be carried out using the manufacturer's protocol. This assay essentially involves the covalent binding of either ymkz5-receptor or a ymkz5-receptor binding partner to a dextran-coated sensor chip which is located in a detector. The test compound and the other complementary protein can then be injected, either simultaneously or sequentially, into the chamber containing the sensor chip either simultaneously or sequentially and the amount of complementary protein that binds can be assessed based on the change in molecular mass which is physically associated with the dextran-coated side of the sensor chip, the change in molecular mass can be measured by the detector system.

In some cases, it may be desirable to evaluate two or more test compounds together for their ability to increase or decrease the formation of a complex between a ymkz5-receptor polypeptide and a ymkz5-receptor binding partner complex In these cases, the assays set forth herein can be readily modified by adding such additional test compound(s) either simultaneous with, or subsequent to, the first test compound. The remainder of steps in the assay are as set forth herein.

In vitro assays such as those described above may be used ad advantageously to screen rapidly large numbers of compounds for effects on complex formation by ymkz5-receptor and ymkz5-receptor binding partner. The assays may be automated to screen compounds generated in phage display, synthetic peptide and chemical synthesis libraries.

Compounds which increase or decrease the formation of a complex between a ymkz5-receptor polypeptide and a ymkz5-receptor binding partner may also be screened in cell culture using cells and cell lines expressing either ymkz5-receptor or ymkz5-receptor binding partner. Cells and cell lines may be obtained from any mammal, but preferably will be from human or other primate, canine, or rodent sources. The binding of an ymkz5-receptor polypeptide to cells expressing ymkz5-receptor binding partner at the surface is evaluated in the presence or absence of test molecules and the extent of binding may be determined by, for example, flow cytometry using a biotinylated antibody to an ymkz5-receptor binding partner. Cell culture assays may be used advantageously to further evaluate compounds that score positive in protein binding assays described herein.

Cell cultures can also be used to screen the impact of a drug candidate. For example, drug candidates may decrease or increase the expression of the ymkz5-receptor gene. In certain embodiments, the amount of ymkz5-receptor polypeptide that is produced may be measured after exposure of the cell culture to the drug candidate. In certain embodiments, one may detect the actual impact of the drug candidate on the cell culture. For example, the overexpression of a particular gene may have a particular impact on the cell culture. In such cases, one may test a drug candidate's ability to increase or decrease the expression of the gene or its ability to prevent or inhibit a particular impact on the cell culture. In other examples, the production of a particular metabolic product such as a fragment of a polypeptide, may result in, or be associated with, a disease or pathological condition. In such cases, one may test a drug canididate's ability to decrease the production of such a metabolic product in a cell culture.

A yeast two hybrid system (Chien et al., Proc. Natl. Acad Sci USA. 88:9578-9583 (1991)) can be used to identify novel polypeptides that bind to, or interact with, ymkz5-receptor polypeptides As an example, hybrid constructs comprising DNA encoding a cytoplasmic domain of an ymkz5-receptor polypeptide fused to a yeast GAL4-DNA binding domain may be used as a two-hybrid bait plasmid. Positive clones emerging from the screening may be characterized further to identify interacting proteins.

P38 Inhibitors

A new approach to intervention between the extracellular stimulus and the secretion of IL-1 and TNFα from the cell involves blocking signal transduction through inhibition of a kinase which lies on the signal pathway. One example is through inhibition of P-38 (also called “RK” or “SAPK-2”. Lee et al., Nature, 372:739 (1994)), a known ser/thr kinase (clone reported in Han et al., Biochimica Biophysica Acta, 1265:224-227 (1995)). A linear relationship has been shown for effectiveness in a competitive binding assay to P-38, and the same inhibitor diminishing the levels of IL-1 secretion from monocytes following LPS stimulation. Following LPS stimulation of monocytes, the levels of messenger RNA for TNF-α have been shown to increase 100 fold, but the protein levels of TNF-α are increased 10,000 fold. Thus, a considerable amplification of the TNF signaling occurs at the translational level. Following LPS stimulation of monocytes in the presence of a P-38 inhibitor, the levels of mRNA are not affected, but the levels of final TNF protein are dramatically reduced (up to 80-90% depending on the effectiveness of the P-38 inhibitor). Thus, the above experiments lend strong support to the conclusion that inhibition of P-38S leads to diminished translational efficiency. Further evidence that TNFα is under translational control is found in the deletion experiments of Beutler et al. and Lee, wherein segments of 3′ untranslated mRNA (3′ UTR) are removed resulting in high translational efficiency for TNFα. More importantly, the P-38 inhibitors did not have an effect on the level of TNFα (i.e., translational efficiency) when the appropriate segments of TNFAα mRNA are deleted. Thus, the correlative data between the level of binding of inhibitors to P-38S and the diminished IL-1 and TNFα levels following, LPS stimulation with the same inhibitors, plus the above biochemical evidence regarding the effect of P-38 inhibitors on translational efficiency of both TNFα and IL-1 make a strong cause and effect relationship. The role of P-38 in the cell is still being delineated; so therefore, other beneficial effects regarding inflammatory diseases or other disease states obtained from its inhibition maybe forthcoming.

Elevated levels of TNFα and/or IL-1 may contribute to the onset, etiology, or exacerbate a number of disease states, including, but not limited to: rheumatoid arthritis; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; antiviral therapy including those viruses sensitive to TNFα inhibition—HIV-1, HIV-2, HIV-3, cytomegalovirus (CMV), influenza, adenovirus, and the herpes viruses including HSV-1, HSV-2, and herpes zoster; muscle degeneration; cachexia; Reiter's syndrome; type II diabetes; bone resorption diseases; graft vs. host reaction; ischemia reperfusion injury; atherosclerosis; brain trauma; Alzheimer's disease; multiple sclerosis; cerebral malaria; sepsis; septic shock; toxic shock syndrome; fever and mylagias due to infection.

Substituted imidazole, pyrrole, pyridine, pyrimidine and the like compounds have been described for use in the treatment of cytokine mediated diseases by inhibition of proinflammatory cytokines, such as IL-1, IL-6, IL-8 and TNF. Substituted imidazoles for use in the treatment of cytokine mediated diseases have been described in U.S. Pat. No. 5,593,992; WO 93/14081; WO 97/18626; WO 96/21452; WO 96/21654; WO 96/40143; WO 97/05878; WO 97/05878; (each of which is incorporated herein by reference in its entirety). Substituted imidazoles for use in the treatment of inflammation has been described in U.S. Pat. No.3,929,807 (which is incorporated herein by reference in its entirety). Substituted pyrrole compounds for use in the treatment of cytokine mediated diseases have been described in WO 97/05877; WO 97/05878; WO 97/16426; WO 97/16441; and WO 97/16442 (each of which is incorporated herein by reference in its entirety). Substituted aryl and heteroaryl fused pyrrole compounds for use in the treatment of cytokine mediated diseases have been described in WO 98/22457 (which is incorporated herein by reference in its entirety). Substituted pyridine, pyrimidine, pyrimidinone and pyridazine compounds for use in the treatment of cytokine mediated diseases have been described in WO 98/24780: WO 98/24782:WO 99/24404, and WO 99/3448 (each of which is incorporated herein by reference in its entirety).

Internalizing Proteins

The TAT protein sequence (from HIV) can be used to internalize proteins into a cell by targeting the lipid bi-layer component of the cell membrane. See e.g., Falwell et al., Proc. Natl Acad. Sci., 91: 664-668 (1994). For example, an 11 amino acid sequence (YGRKKRRQRRR; SEQ ID NO: 15) of the HIV TAT protein (termed the “protein transduction domain”, or TAT PDT) has been shown to mediate delivery of large bioactive proteins such as β-galactosidase and p27Kip across the cytoplasmic membrane and the nuclear membrane of a cell. See Schwarze et al., Science, 28: 1569-1572 (1999); and Nagahara et al., Nature Medicine, 4: 1449-1452 (1998). Schwartze et al. Science 285: 1569-72 (1999) demonstrated that cultured cells acquired β-gal activity when exposed to a fusion of the TAT PDT and β-galactosidase. Injection of mice with the TAT-β-gal fusion proteins resulted in β-gal expression in a number of tissues, including liver, kidney, lung, heart, and brain tissue.

It will thus be appreciated that the TAT protein sequence may be used to internalize a desired protein or polypeptide into a cell. In the context of the present invention, the TAT protein sequence can be fused to another molecule such as ymkz5-receptor antagonist (i e.: anti-ymkz5-receptor selective binding agent or small molecule) and administered intracellularly to inhibit the activity of the ymkz5-receptor molecule. Where desired, the ymkz5-receptor polypeptide itself, or a peptide fragment or modified form of ymkz5-receptor, may be fused to such a protein transducer for administrating to cells using the procedures, described herein.

ymkz5-Polypeptide Compositions and Administration

Members of the TNF ligand family have been implicated in mediation of a number of diseases. The pleiotropic nature of the TNF and related ligand family prevents generalization about whether it is beneficial or injurious. It is clear that in some instances, the local effects of TNF and other members of the TNF-ligand family cytokines improve host defense mechanisms by mobilizing substrate, increasing immune cell function, stimulating inflammation and in killing cancer cells. However, in other cases the toxicity of TNF and related cytokines may cause disease by mediating shock, tissue injury, or catabolic injury. There are many diseases wherein members of the TNF ligand family mediated injury may be treated or ameliorated by the administration of , soluble forms of the receptor or other ligand binding molecules . These diseases include acquired-immunodeficiency syndrome (AIDS), anemia, autoimmune diseases, cachexia, cancer, cerebral malaria, diabetes mellitus, disseminated intravascular coagulopathy, erythroid sick syndrome, hemorrhage shock, hepatitis, insulin resistance, leprosy, leukemia, lymphoma, meningitis, multiple sclerosis, myocardial ischaemia, obesity, rejection of transplanted organs, rheumatoid arthritis, septic shock syndrome, stroke, adult respiratory distress syndrome (ARDS), tuberculosis, and a number of viral diseases.

Pharmaceutical compositions of ymkz5-receptor polypeptides are within the scope of the present invention for prophylactic and therapeutic treatment of humans and animals for indications resulting from abnormal expression of ymkz5-receptor or where it is determined that administration of ymkz5-receptor polypeptide will result in the amelioration or cure of the indications. Such ymkz5-receptor pharmaceutical compositions may comprise a therapeutically effective amount of a ymkz5-receptor polypeptide and/or its binding partner, or therapeutically active fragments(s), variant(s), or derivative(s) thereof in admixture with a pharmaceutically or physically acceptable additives and/or carriers. Suitable formulation materials or pharmaceutically acceptable agents include, but are not limited to, antioxidants, preservatives, colors, flavoring, and diluting agents, emulsifying agents, suspending agents, solvents, fillers, bulking agents, buffers, delivery vehicles, diluents, excipients, and/or pharmaceutical adjuvants. Typically, a therapeutic compound containing ymkz5-receptor polypeptide(s) will be administered in the form of a composition comprising purified polypeptide, fragment(s), variant(s), or derivative(s) in conjunction with one or more physiologically acceptable carriers, excipients, or diluents. For example, a suitable vehicle may be water for injection, physiological solution, or artificial cerebrospinal fluid possibly supplemented with other materials common in compositions for parenteral delivery.

Neutral buffered saline or saline mixed with serum albumin are exemplary appropriate carriers. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5. or acetate buffer of about pH 4.0-5.5. which may further include sorbitol or a suitable substitute therefor. The pH of the solution should also be selected based on the relative solubility of ymkz5-receptor at various pHs.

The primary solvent in a composition may be either aqueous or non-aqueous in nature. In addition, the vehicle may contain other formulation materials for modifying or maintaining the pH, osmolarity, viscosity, clarity, color, isotonically, sterility, stability, rate of dissolution, or odor of the formulation. Similarly, the composition may contain additional formulation materials for modifying or maintaining the rate of release of ymkz5-receptor protein, or for promoting the absorption or penetration of ymkz5-receptor protein.

Compositions comprising the ymkz5-receptor polypeptide compositions can be administered parentally. Alternatively, the compositions may be administered intravenously or subcutaneously. When systemically administered, the therapeutic compositions for use in this invention may be in the form of a pyrogen-free, parentally acceptable aqueous solution. The preparation of such pharmaceutically acceptable protein solutions, with due regard to pH, isotonicity, stability and the like, is within the skill of the art.

Therapeutic formulations of ymkz5-receptor polypeptide compositions useful for practicing the present invention may be prepared for storage by mixing the selected composition having the desired degree of purity with optional physiologically acceptable carriers, excipients, or stabilizers (Remington's Pharmaceutical Sciences, 18th Edition, A. R. Gennaro, ed., Publishing Company (1990)) in the form of a lyophilized cake or an aqueous solution.

Acceptable carriers, excipients or stabilizers are nontoxic to recipients and are preferably inert at the dosages and concentrations employed, and include buffers (such as borqates, bicarbonate, tris-HCl, phosphates, citrates, or other organic acids); antioxidants (such as ascorbic acid, sodium sulfate or hydosulfite); low molecular weight polypeptides; proteins (such as serum albumin, gelatin, or immunoglobulins); hydrophilic polymers (such as polyvinylpyrrolidone); amino acids (such as glycine, glutamine, asparagine, arginine or lysine); fillers; monosaccharides, disaccharides, and other carbohydrates (such as glucose, mannose, or dextrins), chelating agents (such as EDTA), sugar alcohols (such as mannitol or sorbitol); salt-forming counterions (such as sodium), and/or nonionic surfactants (such as Tween, pluronics or polyethylene glycol (PEG)).

An effective amount of the ymkz5-receptor polypeptide(s) composition to be employed therapeutically will depend, for example, upon the therapeutic objectives such as the indication for which the composition is being used, the route of administration (e g., whether it is administered locally or systemically), and the condition of the patient (e.g., patient's general health, anaureuesis, age, weight, sex). It is essential, when determining the therapeutically effective dose, to take into account the quantity of ymkz5-receptor or other members of the TNF family of ligand secreted which are responsible for the disease as well as the quantity of endogenous ymkz5-receptor. Basically, it can be assumed that for effective treatment of a disease triggered by the secretion of the cytokine(s), at least the same molar amount of the ymkz5-receptor polypeptide(s) is required as quantity of ligand secreted, and possibly a multiple excess might be needed, although less may be needed depending on the nature of the specific ligand involved and the nature of its interaction with ymkz5-receptor. Accordingly, it will be necessary for the therapist to titer the dosage and/or in vivo modify the route of administration as required to obtain the optimal therapeutic effect. A typical daily dosage may range from about 0.mg/kg to up to 100 mg/kg or more, depending on the factors mentioned above. Typically, a clinician will administer the composition until a dosage is reached that achieves the desired effect. The composition may therefore be administered as a singe dose, or as two or more doses (which may or may not contain the same amount of ymkz5-receptor polypeptide) over time, or as a continuous infusion via implantation device or catheter.

An effective amount of an ymkz5-receptor pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the ymkz5-receptor molecule is being used, the route of administration, and the size (body weight, body surface or organ size) and condition (the age and general health) of the patient. Accordingly, the clinician may titer the dosage and modify the route of administration to obtain the optimal therapeutic effect.

The ymkz5-receptor polypeptide composition to be used for in vivo administration must be sterile. This is readily accomplished by filtration through sterile filtration membranes. Where the composition is lyophilized sterilization using these methods may be conducted either prior to or following lyophilization and reconstitution. The composition for parenteral administration ordinarily will be stored in lyophilized form or in solution.

Therapeutic compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsions solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

Effective administration forms, such as (1) slow-release formulations, (2) inhalant mists, or (3) orally active formulations are also envisioned. Pharmaceutical composition comprising therapeutically effective dose of the ymkz5-receptor polypeptide also may be formulated for parenteral administration. Such parenterally administered therapeutic compositions are typically in the form of a pyrogen-free, parenterally acceptable aqueous solution comprising ymkz5-receptor in a pharmaceutically acceptable vehicle. The ymkz5-receptor pharmaceutical compositions also may include particulate preparations of polymeric compounds such as polylactic acid, polyglycolic acid, etc. or the introduction of ymkz5-receptor into liposomes. Hyaluronic acid may also be used, and this may have the effect of promoting sustained duration in the circulation.

When parenteral administration is contemplated, the therapeutic compositions for use in this invention may be in the form of a pyrogen-free, parenterally acceptable aqueous solution comprising the desired ymkz5-receptor molecule in a pharmaceutically acceptable vehicle. A particularly suitable vehicle for parenteral injection is sterile distilled water in which an ymkz5-receptor molecule is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), or beads, or liposomes, that provides for the controlled or sustained release of the product which may then be delivered as a depot injection. Hyaluronic acid may also be used, and this may have the effect of promoting sustained duration in the circulation. Other suitable means for the introduction of the desired molecule include implantable drug delivery devices.

The preparations of the present invention may include other components, for example parenterally acceptable preservatives, tonicity agents, cosolvents, wetting agents, complexing agents, buffering agents, antimicrobials, antioxidants and surfactants, as are well known in the art. For example, suitable tonicity enhancing agents include alkali metal halides (preferably sodium or potassium chloride), mannitol, sorbitol and the like. Suitable preservatives include, but are not limited to, benzalkonium chloride, thimerosal, phenethyl alcohol, methylparaben, propylparaben, chlorhexidine, sorbic acid and the like. Hydrogen peroxide may also be used as preservative. Suitable cosolvents are for example glycerin, propylene glycol and polyethylene glycol. Suitable complexing, agents are for example caffeine, polyvinylpyrrolidone, beta-cyclodextrin or hydroxypropyl-beta-cyclodextrin. Suitable surfactants or wetting agents include sorbitan esters, polysorbates such as polysorbate 80, tromethamine, lecithin, cholesterol, tyloxapal and the like. The buffers call be conventional buffers such as borate, citrate, phosphate, bicarbonate, or Tris-HCl.

The formulation components are present in concentration that are acceptable to the site of administration. For example, buffers are used to maintain the composition at physiological pH or at slightly lower pH, typically within a pH range of from about 5 to about 8.

In one embodiment, a pharmaceutical composition may be formulated for inhalation. For example, ymkz5-receptor may be formulated as a dry powder for inhalation. Ymkz-receptor polypeptide or ymkz5-receptor polynucleotide inhalation solutions may also be formulated with a propellant for aerosol delivery. In yet another embodiment, solutions may be nebulized. Pulmonary administration is further described in PCT application No. US/US94/001875 which describes pulmonary delivery of chemically modified proteins.

It is also contemplated that certain formulations containing ymkz5-receptor may be administered orally. The ymkz5-receptor which is administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of the receptor polypeptide. Diluents, flavorings, low melting point waxes, vegetable oils, lubricants, suspending agents, tablet disintegrating agents, and binders may also be employed.

Another pharmaceutical composition may involve an effective quantity of ymkz5-receptor in a mixture with non-toxic excipients which are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional ymkz5-receptor pharmaceutical composition will be evident to those skilled in the art, including formulations involving ymkz5-receptor in sustained- or controlled-release delivery formulation. Techniques for formulating a variety of other sustained- or controlled-delivery means, such as liposome carriers, bio-erodible microparticles or porous beads and depot injections, are also known to those skilled in the art. See, for example. PCT/US93/00829 which describes controlled of release porous polymeric microparticles for the delivery of pharmaceutical compositions. additional examples of sustained release preparations include semipermeable polymer materials in the form of shaped articles e.g., film or microcapsules.

Regardless of the manner of administration, the specific dose may be calculated according to body weight, body surface area or organ size. Further refinement of the calculations necessary to determine the appropriate dosage for treatment involving each of the above mentioned formulations is routinely made by those of ordinary skill in the art and is within the gambit of tasks routinely performed by them. Appropriate dosages may be ascertained through use of appropriate dose-response data. A typical dosage may range from about 0.1 mg/kg to up to about 100 mg/kg or more, depending, on the factors mentioned above. In other embodiments, the dosage may range from 0.1 mg/kg up to about 100 mg/kg; or 1 mg/kg up to about 100 mg/kg; or 5 mg/kg up to about 100 mg/kg.

The frequency of dosing will depend upon the pharmacokinetic parameters of the ymkz5-receptor molecule in the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the composition is in accord with known methods, e.g. oral, injection or infusion by intravenous, intraperitoneal, intracerebral (intraparenchymal), intraventricular, intramuscular, intraocular, intraarterial, or intralesional routes, or by sustained release systems or implantation device which may optionally involve the use of a catheter. Where desired, the compositions may be administered continuously by infusion, bolus injection or by implantation device. Alternatively or additionally, the composition may be administered locally via implantation into the affected area of a membrane, sponge, or other appropriate material on to which ymkz5-receptor polypeptide has been absorbed.

One may further administer the present pharmaceutical compositions by pulmonary administration, see, e.g., International Publication No: WO 94/20069, which discloses pulmonary delivery of chemically modified proteins, herein incorporated by reference. For pulmonary delivery, the particle size should be suitable for delivery to the distal lung. For example, the particle size may be from 1 mm to 5 mm, however, larger particles may be used, for example, if each particle is fairly porous. Alternatively or additionally, the composition may be administered locally via implantation into the affected area of a membrane, sponge, or other appropriate material on to which receptor polypeptide has been absorbed or encapsulated. Where an implantation de ice is used, the device may be implanted into any suitable tissue or organ, and delivery may be directly through the device via bolus, or via continuous administration, or via catheter using continuous infusion.

Ymkz5-receptor polypeptide and/or its binding partner may also be administered in a sustained release formulation or preparation. Suitable polymer compositions preferably have intrinsic and controllable biodegradability so that they persist for about a week to about six months; are non-toxic containing no significant toxic monomers and degrading into non-toxic components: are biocompatible, are chemically compatible with substances to be delivered, and tend not to denature the active substance; are sufficiently porous to allow the incorporation of biologically active molecules and their subsequent liberation from the polymer by diffusion, erosion or a combination thereof; are able to remain at the site of the application by adherence or by geometric factions, such as being formed in place or softened and subsequently molded or formed into microparticles which are trapped at a desired location; are capable of being delivered by techniques of minimum invasivity such as by catheter, laparoscope or endoscope. Sustained release matrices include polyesters, hydrogels, polylactides (U.S. Pat. No. 3,773,919. EP 58,481), copolymers of L-glutamic acid and gamma ethyl-L-glutamate (Sidman et al. Biopolymers, 22: 547-556 (1983,)), poly (2-hydroxyethyl-methacrylate) (Langer et al., J. Biomed. Mater. Res., 15: 167-277 (1981) and Langer, Chem. Tech., 12: 98-105 (1982)), ethylene vinyl acetate (Langer et al., supra ) or poly-D(-)-3-hydroxybutyric acid (EP 133,988). Sustained-release compositions also may include liposomes, which can be prepared by any of several methods known in the art (e g., Eppstein et al , Proc. Natl. Acad. Sci. USA, 82: 3688-3692 (1985); EP 36,676; EP 88,046; EP 143,949, incorporated herein by reference).

The ymkz5-receptor polypeptides, variants, derivatives or fragments thereof, may be employed alone, together, or in combination with other pharmaceutical compositions. The ymkz5-receptor polypeptides, fragments, variants, and derivatives may be used in combination with cytokines, cytokine inhibitors, growth factors, antibiotics, anti-inflammatories, and/or chemotherapeutic agents as is appropriate for the indication being treated

In some cases, it may be desirable to use ymkz 5-receptor polpeptide compositions in an ex vivo manner. Here, cells, tissues, or organs that have been removed from the patient are exposed to ymkz5-receptor polpeptide compositions after which the cells, tissues and/or organs are subsequently implanted back into the patient.

In other cases, a ymkz5-receptor polypeptide can be delivered by implanting into patients certain cells that have been genetically engineered, using methods such as those described herein, to express and secrete the polypeptides, fragments, variants, or derivatives. Such cells may be animal or human cells, and may be autologous, heterologous, or xenogeneic. Optionally, the cells may be immortalized. In order to decrease the chance of an immunological response, it is preferred that the cells may be encapsulated to avoid infiltration of surrounding tissues. The encapsulation materials are typically biocompatible, semi-permeable polymeric enclosures or membranes that allow release of the protein product(s) but prevent the destruction of the cells by the patient's immune system or by other detrimental factors from the surrounding tissues.

Methods used for membrane encapsulation of cells are familiar to the skilled artisan, and preparation of encapsulated cells and their implantation in patients may be accomplished without undue experimentation. See, e.g., U.S. Pat. Nos. 4,892,538; 5,011,477; and 5,106,627, incorporated herein by reference. A system for encapsulating living cells is described in International Publication No: WO 91/10425 (Aebischer et al.). Techniques for formulating a variety of other sustained or controlled delivery means, such as liposome carriers, bio-erodible particles or beads, are also known to those in the art, and are described, for example, in U.S. Pat. No. 5,653,375, incorporated herein by reference. The cells, with or without encapsulation, may be implanted into suitable body tissues or organs of the patient.

As discussed above, it may be desirable to treat isolated cell populations such as stem cells, lymphocytes, red blood cells, chondrocytes, neurons, and the like; add as appropriate with one or more ymkz5-receptor polypeptides, variants, derivatives and/or fragments. This can be accomplished by exposing the isolated cells to the polypeptide, variant, derivative, or fragment directly, where it is in a form that is permeable to the cell membrane.

The present invention relates to improved methods for both the in vitro production of therapeutic proteins and for the production and delivery of therapeutic proteins by gene therapy.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally silent ymkz5-receptor gene, or an under expressed gene, and thereby produce a cell which expresses therapeutically efficacious amounts of ymkz5-receptor polypeptides.

Homologous Recombination

It is further envisioned that ymkz5-receptor protein may be produced by homologous recombination, or with recombinant production methods utilizing control elements introduced into cells already containing DNA encoding ymkz5-receptor. For example, homologous recombination methods may be used to modify a cell that contains a normally transcriptionally silent ymkz5-receptor gene, or an under expressed gene, and thereby produce a cell which expresses therapeutically efficacious amounts of ymkz5-receptor. Homologous recombination is a technique originally developed for targeting genes to induce or correct mutations in transcriptionally active genes (Kucherlapati, R. Prog. in Nucl. Acid Res. and Mol Biol., 36:301, (1989)). The basic technique was developed as a method for introducing specific mutations into specific regions of the mammalian genome (Thomas et al., Cell, 44:419-428 (1986); Thomas and Capecchi, Cell, 51:503-512 (1987); Doetschman et al., Proc. Natl. Acad. Sci. 85:8583-8587 (1988)) or to correct specific mutations within defective genes (Doetschman et al., Nature, 330:576-578 (1987)). Exemplary homologous recombination techniques are described in U.S. Pat. No. 5,272,071, EP Publication No: 91 93 051, EP Publication No. 505 500; PCT/US90/07642, International Publication No: WO 91/09955, incorporated herein by reference.

Through homologous recombination, the DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process. It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome.

Attached to these pieces of targeting DNA are regions of DNA which may interact with or control the expression of a ymkz5-receptor polypeptide, e.g., flanking sequences. For example, a promoter/enhancer element, a suppresser, or an exogenous transcription modulatory element is inserted in the genome of the intended host cell in proximity and orientation sufficient to influence the transcription of DNA encoding the desired ymkz5-receptor polypeptide. The control element controls a portion of the DNA present in the host cell genome. Thus, the expression of ymkz5-receptor protein may be achieved not by transfection of DNA that encodes the ymkz5-receptor gene itself, but rather by the use of targeting DNA (containing regions of homology with the endogenous gene of interest) coupled with DNA regulatory segments that provide the endogenous gene sequence with recognizable signals for transcription of a ymkz5-receptor protein.

In an exemplary method, expression of a desired targeted gene in a cell (i.e., a desired endogenous cellular gene) is altered via homologous recombination into the cellular genome at a preselected site, by the introduction of DNA which includes at least a regulatory sequence, an exon and a splice donor site. These components are introduced into the chromosomal (genomic) DNA in such a manner that this, in effect, results in the production of a new transcription unit (in which the regulatory sequence, the exon and the splice donor site present in the DNA construct are operatively linked to the endogenous gene). As a result of the introduction of these components into the chromosomal DNA, the expression of the desired endogenous gene is altered.

Altered gene expression, described herein, encompasses activating (or causing to be expressed) a gene which is normally silent (unexpressed) in the cell as obtained, as well as increasing expression of a gene which is not expressed at physiologically significant levels in the cell as obtaine. The embodiments further encompass changing the pattern of regulation or induction such that it is different from the pattern of regulation or induction that occurs in the cell as obtained, and reducing (including eliminating) expression of a gene which is expressed in the cell as obtained.

One method by which homologous recombination can be used to increase, or cause, ymkz5-receptor polypeptide production from a cell's endogenous ymkz5-receptor like gene involves first using homologous recombination to place a recombination sequence from a site-specific recombination system (e.g., Cre/loxP, FLP/FRT) (Sauer, Current Opinion In Biotechnology, 5:521-527, (1994); Sauer, Methods in Enzymology, 225:890-900, (1993)) upstream (that is, 5′ to) of the cell's endogenous genomic ymkz5-receptor like polypeptide coding region. A plasmid containing a recombination site homologous to the site that was placed just upstream of the genomic ymkz5-receptor polypeptide coding region is introduced into the modified cell line along with the appropriate recombinase enzyme. This recombinase causes the plasmid to integrate, via the plasmid's recombination site, into the recombination site located just upstream of the genomic ymkz5-receptor polypeptide coding region in the cell line (Baubonis and Sauer, Nucleic Acids Res., 21:2025-2029, (1993); O'Gorman et al., Science, 251:1351-1355, (1991)). Any flanking sequences known to increase transcription (e.g., enhancer/promoter, intron, translational enhancer), if properly positioned in this plasmid, would integrate in such a manner as to create a new or modified transcriptional unit resulting in de novo or increased ymkz5-receptor polypeptide production from the cell's endogenous ymkz5-receptor gene.

A further method to use the cell line in which the site specific recombination sequence had been placed just upstream of the cell's endogenous genomic ymkz5-receptorpolypeptide coding region is to use homologous recombination to introduce a second recombination site elsewhere in the cell line's genome. The appropriate recombinase enzyme is then introduced into the two-recombination-site line, causing a recombination event (deletion, inversion, translocation ) (Sauer, Current Opinion In Biotechnology, supra, (1994); Sauer, Methods in Enzymology, supra, (1993)) that would create a new or modified transcriptional unit resulting in de novo or increased ymkz5-receptor polypeptide production from the cell's endogenous ymkz5-receptor gene.

An additional approach for increasing, or causing, the expression of ymkz5-receptor polypeptide from a cell's endogenous ymkz5-receptor gene involves increasing, or causing, the expression of a gene or genes (e.g., transcription factors) and/or decreasing the expression of a gene or genes (e.g., transcriptional repressors) in a manner which results in de novo or increased ymkz5-receptor polypeptide production from the cell's endogenous ymkz5-receptor gene. This method includes the introduction of a non-naturally occurring polypeptide (e.g., a polypeptide comprising a site specific DNA binding domain fused to a transcriptional factor domain) into the cell such that de novo or increased ymkz5-receptor polypeptide production from the cell's endogenous ymkz5-receptor gene results.

The present invention further relates to DNA constructs useful in the method of altering expression of a target gene. In certain embodiments, the exemplary DNA constructs comprise: (a) one or more targeting sequences, (b) a regulatory sequence; (c) an exon; and (d) an unpaired splice-donor site. The targeting, sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site In the cellular chromosomal DNA with which homologous recombination is to occur. In the construct, the exon is generally 3′ of the regulatory sequence and the splice-donor site is 3′ of the exon. If the sequence of a particular gene is known, such as the nucleic acid sequence of ymkz5-receptor presented herein, a piece of DNA that is complementary to a selected region of the gene can be synthesized or otherwise obtained, such as by appropriate restriction of the native DNA at specific recognition sites bounding the region of interest This piece serves as a targeting, sequence upon insertion into the cell and will hybridize to its homologous region within the genome. If this hybridization occurs during DNA replication, this piece of DNA, and any additional sequence attached thereto, will act as an Oka/aki fragment and will be backstitched into the newly synthesized daughter strand of DNA The present invention, therefore, includes nucleotides encoding a ymkz5-receptor molecule, which nucleotides may be used as targeting sequences.

Ymkz5-receptor Cell Therapy and Gene Therapy

Ymkz5-receptor cell therapy, e.g., the implantation of cells producing ymkz5-receptor, is also contemplated. This embodiment would involves implanting cells capable of synthesizing and secreting a biologically active form of the soluble ymkz5-receptor. Such soluble ymkz5-receptor-producing cells can be cells that are natural producers of ymkz5-receptor polypeptides or may be recombinant cells whose ability to produce ymkz5-receptor has been augmented by transformation with a gene encoding the desired ymkz5-receptor molecule or with a gene augmenting the expression of ymkz5-receptor. Such a modification may be accomplished by means of a vector suitable for delivering the gene as well as promoting its expression and secretion. In order to minimize a potential immunological reaction in patients being administered a ymkz5-receptor polypeptide, as may occur with the administration of a or polypeptide of a foreign species, it is preferred that the natural cells producing ymkz5-receptor be of human origin and produce human ymkz5-receptor polypeptide. Likewise, it is preferred that the recombinant cells producing ymkz5-receptor polypeptides be transformed with an expression vector containing a gene encoding a human ymkz5-receptor polypeptide.

Implanted cells may be encapsulated to avoid infiltration of surrounding tissue. Human or non-human animal cells may be implanted in patients in biocompatible, semipermeable polymeric enclosures or membranes that allow release of ymkz5-receptor but that prevent destruction of the cells by the patient's immune system or by other detrimental factors from the surrounding tissue. Alternatively, the patient's own cells, transformed to produce ymkz5-receptor ex vivo, could be implanted directly into the patient without such encapsulation.

Techniques for the encapsulation of living cells are known the art, and the preparation of the encapsulated cells and their implantation in patients may be routinely accomplished without undue experimentation. For example, Baetge et al describes membrane capsules containing genetically engineered cells for the effective delivery of biologically active molecules. The capsules are biocompatible and are easily retrievable The capsules encapsulate cells transfected with recombinant DNA molecules comprising DNA sequences coding for biologically active molecules operatively linked to promoters that are not subject to down regulation in vivo upon implantation. The devices provide for the delivery of the molecules from living cells to specific sites within a recipient. See U.S. Pat. Nos. 4,892,538, 5,011,472, and 5,106,627, incorporated herein by reference. A system for encapsulating living cells is described in Aebischer et al. (WO 91/10425, WO 91/10470); Winn et al., Exper. Neurol., 113:322-329, (1991), Aebischer et al., Exper. Neurol., 111:269-275, (1991); and Tresco et al., ASAIO, 38:17-23, (1992).

In vivo and in vitro gene therapy delivery of ymkz5-receptor is also encompassed by the present invention. In vivo gene therapy may be accomplished by introducing the gene encoding ymkz5-receptor into cells via local injection of a polynucleotide molecule or other appropriate delivery vectors. (Hefti, J. Neurology, 25:1418-1435, (1994)). For example, a polynucleotide molecule encoding ymkz5-receptor may be contained in an adeno-associated virus vector for delivery to the targeted cells (e.g., Johnson, International Publication No. WO 95/34670; International Application No. PCT/US95/07178). The recombinant adeno-associated virus (AAV) genome typically contains AAV inverted terminal repeats flanking a DNA sequence encoding ymkz5-receptor operably linked to functional promoter and polyadenylation sequences.

Alternative viral vectors include, but are not limited to, retrovirus, adenovirus, herpes simplex virus and papilloma virus vectors. U.S. Pat. No. 5,672,344 (issued Sept. 30, 1997, Kelley, et al., University of Michigan) describes an in vivo viral-mediated gene transfer system involving a recombinant neurotrophic HSV-1vector. U.S. Pat. No. 5,399,346 (issued Mar. 21, 1995, Anderson et al., Department of Health and Human Services) provides examples of a process for providing a patient with a therapeutic protein by the delivery of human cells which have been treated in vitro to insert a DNA segment encoding therapeutic protein. Additional methods and materials for the practice of gene therapy techniques are described in U.S. Pat. No. 5,631,236 (issued May 20, 1997, Woo et al., Baylor College of Medicine) involving adenoviral sectors: U.S. Pat. No. 5,672,510 (issued Sept. 30, 1997, Eglitis et al., Genetic Therapy, Inc.) involving retroviral vectors; and U.S. Pat. No. 5,635,399 (issued Jun. 3, 1997, Kriegler et al., Chiron Corporation) involving retroviral vectors expressing cytokines.

Nonviral delivery methods include liposome-mediated transfer, naked DNA delivery (direct injection), receptor-mediated transfer (ligand-DNA complex), electroporation, calcium phosphate precipitation and microparticle bombardment (e.g., gene gun). Gene therapy materials and methods may also include inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, transcription factors to enhance expression by a vector as well as methods of vector manufacture. Such additional methods and materials for the practice of gene therapy techniques are described in U.S. Pat. No.4,970,154 (issued Nov. 13, 1990, D. C. Chang, Baylor College of Medicine) electroporation techniques; International Application No. WO 9640958 (published 961219, Smith et al., Baylor College of Medicine) nuclear ligands; U.S. Pat. No. 5,679,559 (issued Oct. 21, 1997, Kim et al., University of Utah Research Foundation) concerning a lipoprotein-containing system for gene delivery; U.S. Pat. No. 5,676,954 (issued Oct. 14, 1997, K. L. Brigham, Vanderbilt University involving liposome carriers; U.S. Pat. No.5,593,875 (issued Jan. 14, 1997, Wurm et al., Genentech, Inc) concerning methods for calcium phosphate transfection; and U.S. Pat. No. 4,945,050 (issued Jul. 31, 1990, Sanford et al., Cornell Research Foundation) wherein biologically active particles are propelled at cells at a speed whereby the particles penetrate the surface of the cells and become incorporated into the interior of the cells.

In yet other embodiments, regulatory elements can be included for the controlled expression of the ymkz5-receptor gene in the target cell. Such elements are turned on in response to an appropriate effector. In this way, a therapeutic polypeptide can be expressed when desired. One conventional control means involves the use of small molecule dimerizers or rapalogs (as described in WO9641865 (PCT/US96/09946). WO9731898 (PCT/US97/03137) and WO9731899 (PCT/US95/03157 )) used to dimerize chimeric proteins which contain a small molecule-binding domain and a domain capable of initiating biological process, such as a DNA-binding protein or transcriptional activation protein. The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain which results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell. See, Science 287:816-817, and 826-830 (2000).

Other suitable control means or gene switches include, but are not limited to, the following systems. Mifepristone (RU486) is used as a progesterone antagonist. The binding of a modified progesterone receptor ligand-binding domain to the progesterone antagonist activates transcription by forming a dimer of two transcription factors which then pass into the nucleus to bind DNA. The ligand binding domain is modified to eliminate the ability of the receptor to bind to the natural ligand. The modified steroid hormone receptor system is further described in U.S. Pat. No. 5,364,791; WO9640911, and WO9710337.

Yet another control system uses ecdysone (a fruit fly steroid hormone) which binds to and activates an ecdysone receptor (cytoplasmic receptor). The receptor then translocates to the nucleus to bind a specific DNA response element (promoter from ecdysone-responsive gene). The ecdysone receptor includes a transactivation domain/DNA-binding domain/ligand-binding domain to initiate transcription. The ecdysone system is further described in U.S. Pat. No. 5,514,578; WO9738117; WO9637609; and WO9303162.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide which activates transcription. Such systems are described in U.S. Pat. Nos. 5,464,758; 5,650,298 and 5,654,168.

Additional expression control systems and nucleic acid constructs are described in U.S. Pat. Nos. 5,741,679 and 5,834,186, to Innovir Laboratories Inc.

It is also contemplated that ymkz5-receptor gene therapy or cell therapy can further include the delivery of a second protein. For example, the host cell may be modified to express and release soluble forms of both ymkz5-receptor and α, or ymkz5-receptor and IL-1R. Alternatively, the ymkz5-receptor and α, or ymkz5-receptor and IL-1R, may be expressed in and released from separate cells. Such cells may be separately introduced into the patient or the cells may be contained in a single implantable device, such as the encapsulating membrane described above.

One manner in which gene therapy can be applied is to use the ymkz5-receptor gene (either genomic DNA, cDNA, and/or synthetic DNA encoding a ymkz5-receptor polypeptide, or a fragment, variant, or derivative thereof) which may be operably linked to a constitutive or inducible promoter to form a “gene therapy DNA construct”. The promoter may be homologous or heterologous to the endogenous ymkz5-receptor gene, provided that it is active in the cell or tissue type into which the construct will be inserted. Other components of the gene therapy DNA construct may optionally include, as required, DNA molecules designed for site-specific integration (e.g., endogenous flanking sequences useful for homologous recombination), tissue-specific promoter, enhancer(s) or silencer(s), DNA molecules capable of providing a selective advantage over the parent cell, DNA molecules useful as labels to identify transformed cells, negative selection systems, cell specific binding agents (as, for example, for cell targeting) cell-specific internalization factors, and transcription factors to enhance expression by a vector as well as factors to enable vector manufacture.

This gene therapy DNA construct can then be introduced into the patient's cells (either ex vivo or in vivo). One means for introducing the gene therapy DNA construct is via viral vectors. Suitable viral vectors typically used in gene therapy for delivery of gene therapy DNA constructs include, without limitation, adenovirus, adeno-associated virus, herpes simplex virus, lentivirus, papilloma virus, and retrovirus vectors. Some of these vectors, such as retroviral vectors, will deliver the gene therapy DNA construct to the chromosomal DNA of the patient's cells, and the gene therapy DNA construct can integrate into the chromosomal DNA; other vectors will function as episomes and the gene therapy DNA construct will remain in the cytoplasm. The use of gene therapy vectors is described, for example, in U.S. Pat. Nos. 5,672,344; 5,399,346; 5,631,236; and 5,635,399, incorporated herein by reference.

Alternative means to deliver gene therapy DNA constructs to a patient's cells without the use of viral vectors include, without limitation, liposome-mediated transfer, direct injection of naked DNA, receptor-mediated transfer (ligand-DNA complex), electroporation, calcium phosphate precipitation, and microparticle bombardment (e.g., “gene gun”). See U.S. Pat. No. 4,970,154; International Application No. WO 96/40958; U.S. Pat. No. 5,679,559; U.S. Pat. No. 5,676,954; and U.S. Pat. No. 5,593,875, incorporated herein by reference.

Another means to increase endogenous ymkz5-receptor polypeptide expression in a cell via gene therapy is to insert one or more enhancer elements into the ymkz5-receptor polypeptide promoter, where the enhancer element(s) can serve to increase transcriptional activity of the ymkz5-receptor polypeptides gene. The enhancer element(s) used will be selected based on the tissue in which one desires to activate the gene(s); enhancer elements known to confer promoter activation in that tissue will be selected. For example, if a ymkz5-receptor polypeptide is to be “turned on” in T-cells, the lck promoter enhancer element may be used. Here, the functional portion of the transcriptional element to be added may be inserted into a fragment of DNA containing the ymkz5-receptor polypeptide promoter (and optionally vector, 5′ and/or 3′ flanking sequence, etc.) using standard cloning techniques. This construct, known as a “homologous recombination construct”, can then be introduced into the desired cells either ex vivo or in vivo.

Gene therapy also can be used to decrease ymkz5-receptor polypeptide expression by modifying the nucleotide sequence of the endogenous promoter(s). Such modification is typically accomplished via homologous recombination methods. For example, a DNA molecule containing all or a portion of the promoter of the ymkz5-receptor gene(s) selected for inactivation can be engineered to remove and/or replace pieces of the promoter that regulate transcription. For example, the TATA box and/or the binding site of a transcriptional activator of the promoter may be deleted using standard molecular biology techniques; such deletion can inhibit promoter activity thereby repressing transcription of the corresponding ymkz5-receptor gene. The deletion of the TATA box or transcription activator binding site in the promoter may be accomplished by generating a DNA construct comprising all or the relevant portion of the ymkz5-receptor polypeptide promoter(s) (from the same or a related species as the ymkz5-receptor gene(s) to be regulated) in which one or more of the TATA box and/or transcriptional activator binding site nucleotides are mutated via substitution, deletion and/or insertion of one or more nucleotide. As a result, the TATA box and/or activator binding site has decreased activity or is rendered completely inactive. This construct, which also will typically contain at least about 500 bases of DNA that correspond to the native (endogenous) 5′ and 3′ DNA sequences adjacent to the promoter segment that has been modified. The construct may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

Other gene therapy methods may also be employed where it is desirable to inhibit the activity of one or more ymkz5-receptor polypeptides. For example, antisense DNA or RNA molecules, which have a sequence that is complementary to at least a portion of the selected ymkz5-receptor polypeptide gene(s) can be introduced into the cell. Typically, each such antisense molecule will be complementary to the start site (5′ end) of each selected ymkz5-receptor gene. When the antisense molecule then hybridizes to the corresponding ymkz5-receptor mRNA, translation of this mRNA is prevented or reduced. Antisense inhibitors provide information relating to the decrease or absence of ymkz5-receptor polypeptides in a cell or organism.

Alternatively, gene therapy may be employed to create a dominant-negative inhibitor of one or more ymkz5-receptor polypeptides. In this situation, the DNA encoding a mutant full length or truncated polypeptide of each selected ymkz5-receptor polypeptide can be prepared and introduced into the cells of a patient using either viral or non-viral methods as described herein. Each such mutant is typically designed to compete with endogenous polypeptide in its biological role.

In addition, an ymkz5-receptor polypeptide, whether biologically active or not, may be used as an immunogen, that is, the polypeptide contains at least one epitope to which antibodies may be raised. Selective binding agents that bind to an ymkz5-receptor polypeptide (as described herein) may be used for in vivo and in vitro diagnostic purposes, including, but not limited to, use in labeled form to detect the presence of ymkz5-receptor polypeptide in a body fluid or cell sample. The antibodies may also be used to prevent, treat, or diagnose a number of diseases and disorders, including those recited herein. The antibodies may bind to an ymkz5-receptor polypeptide so as to diminish or block at least one activity characteristic of an ymkz5-receptor polypeptide, or may bind to a polypeptide to increase at least one activity characteristic of an ymkz5-receptor polypeptide (including by increasing the pharmacokinetics of the ymkz5-receptor polypeptide).

Additional Uses of ymkz5 like Nucleic Acids and Polypeptides

Nucleic acid molecules of the present invention (including those that do not themselves encode biologically active polypeptides) may be used to map the locations of the ymkz5-receptor gene and related genes on chromosomes. Mapping may be done by techniques known in the art, such as PCR amplification and in situ hybridization.

Ymkz5-receptor nucleic acid molecules (including those that do not themselves encode biologically active polypeptides), may be useful as hybridization probes in diagnostic assays to test, either qualitatively or quantitatively, for the presence of an ymkz5-receptor DNA or corresponding RNA in mammalian tissue or bodily fluid samples. The ymkz5-receptor polypeptides may be used (simultaneously or sequentially) in combination with one or more cytokines, growth factors, antibiotics, anti-inflammatories, and/or chemotherapeutic agents as is appropriate for the indication being treated.

Other methods may also be employed where it is desirable to inhibit the activity of one or more ymkz5-receptor polypeptides. Such inhibition may be effected by nucleic acid molecules which are complementary to and hybridize to expression control sequences (triple helix formation) or to ymkz5-receptor mRNA. For example, antisense DNA or RNA molecules which have a sequence that is complementary to at least a portion of the selected ymkz5-receptor gene(s) can be introduced into the cell. Anti-sense probes may be designed by available techniques using the sequence of ymkz5-receptor polypeptide disclosed herein. Typically, each such antisense molecule will be complementary to the start site (5′ end) of each selected ymkz5-receptor gene. When the antisense molecule then hybridizes to the corresponding ymkz5-receptor mRNA, translation of this mRNA is prevented or reduced. Anti-sense inhibitors provide information relating to the decrease or absence of an ymkz5-receptor polypeptide in a cell or organism.

The following examples are intended for illustration purposes only, and should not be construed as limiting the scope of the invention in any way

### EXAMPLE 1

### Isolation of Murine ymkz5-Receptor Gene Using Δ kFGF-Signal Trap Method

Secretion signal trap method is a novel way to clone 5′ ends of cDNAs encoding secreted proteins from a random cDNA library. Generally, signal trapping relies on the secretion of a reporter polypeptide by signal sequences present in a cDNA library. The secreted reporter polypeptide may be detected by a variety of assays based upon growth selection, enzymatic activity or immune reactivity. (See U.S. Pat. No. 5,536,637; Klein et al., Proc. Natl. Acad. Sci. USA, 93:7108-7113 (1996); Imai et al., J. Biol. Chem., 271:21514-21521 (1996)). Published PCT application No. WO 96/409904 describes signal trap cloning by selection for growth-factor dependent cell lines.

In the instant case, a novel method for trapping signal sequence DNA from cDNA libraries was utilized to isolate and identify novel secreted proteins, including ymkz5-receptor polypeptide. In the instant case a modified kFGF7 signal trap vector, containing DNA encoding a reporter polypeptide lacking functional 5′-signal sequences and designated ΔkFGF7, was utilized in generating a cDNA library from a desired cell source. Secretion of the reporter polypeptide is indicative of the presence of functional signal sequence and may be detected by a variety of methods including growth under certain conditions, enzyme activity or immune reactivity. Significantly, the molecule of the present invention, ymkz5-receptor polypeptide, was identified utilizing the method described below for selecting signal sequences in mammalian cells (NIH 3T3 cells) using a reporter polypeptide (kFGF) which stimulated the growth of host cells.

Murine ymkz5 cDNA was isolated from a E11 mouse placental cDNA library that was made using the KFGF signal trap vector, ΔkFGF4 described above. Briefly, poly A+RNA was prepared from mouse placenta using a commercially available RNA extraction kit and mRNA purification kit (Pharmacia Biotech). The cDNA library was made following the protocol of SuperScript™ Plasmid System for cDNA synthesis and Plasmid Cloning (GIBCO/BRL, Cat. No. 18248-013) with some modification. To make cDNA with random 3′ ends followed by a Not I site, the oligonucleotide 1360-38: GGA AGG AAA AAA GCG GCC GCA ACA NNN NNN NNN (SEQ ID NO: 1) was made and used as the primer for first strand DNA synthesis. Five μg of poly A RNA and 0.5 μg of the primer was used in the first strand reaction. After second strand synthesis using published procedures, Sal I adapter ligation, and Not I digestion, cDNA was purified using a mini Q column and FPLC (Pharmacia). The cDNA was adjusted to buffer A, 0.6M NaCl, 20mM Tris pH 8.0, and loaded on the column. The column was washed with 3 ml of buffer A at 0.1 ml/minute. The bound cDNA was eluted with 0.6 ml of buffer B, 20 mM Tris pH8.0, 7.5 M NaCl. The solution was divided into two 1.5 ml tubes. To each tube was added 2.5 μg yeast tRNA, 150 μl of 7.5 M NHOAc, and 900 μl ethanol. The cDNA was precipitated, pelleted by centrifugation at 1400 rpm for 20 minutes, and washed with 0.5 ml of 70% ethanol.

The cDNA prepared in this manner was ligated into the Sal I and Not I digested vector, ΔkFGF4 wherein the cDNA fragments were ligated to a kFGF gene lacking the 5′-signal peptide sequence. The ligation was carried in 20 μl containing 75 ng of vector DNA, 20 ng of cDNA, 1×ligase buffer, and 1 μl of T4 ligase at 16° C. for 20 hours. The ligated DNA was precipitated, and introduced into E. coli by electroporation as described in the protocol. The transformed bacteria cells were grown in 5 ml SOC at 37° C. for 1 hour, and then frozen at −80° C. with 10% glycerol.

The isolation and identification of novel cDNAs using kFGF signal trapping method was based on the observation that NIH/3T3 cells transfected with kFGF signal trap vectors containing test DNA fragments and signal sequences continued to grow and form colonies in selection medium while NIH/3T3 cells transfected with employ vectors or untransfected NIH/3T3 cells did not grow in the selection medium.

Plasmid DNA from the cDNA library was prepared in pools of 50,000 colony forming units (cfu) each. E. coli transformed with a cDNA library in the ΔkFGF4 signal trap vector were plated on 150 mm LB agar plates with 100 μg/ml ampicillin and incubated at 37° C. overnight. About 50,000 colony forming units (cfu) from agar plates were pooled into 50 ml LB in a 250 ml flask. The bacteria were grown for 3 hours with agitation, and pelleted by centrifugation at 4000 rpm for 10 minutes in 50 ml conical tubes. Ten pools were prepared. Plasmid DNA was isolated from the pools using QIAGEN maxi prep.

Plasmid DNA was introduced into NIH 3T3 cells by the standard calcium phosphate transfection as previously described (Sambrook et al, supra). Briefly, 100 ng of each cDNA library pool was used to transfect about 200,000 cells in one 35 mm plate. After 24 hours, the cells from one 35 mm plate were split into five 100 mm plates and grown in normal medium for one day followed by low serum medium for 13 days. About 2000 colonies grew from transfected cells after the two week incubation in the selection medium. These colonies were then analyzed for novel genes that encoded secreted polypeptides as described below.

To each 100 mm tissue culture plate was added 2 ml of trypsin-EDTA followed by incubation at 37° for 5 minutes. The cells in the colonies were released from the surface of the plate by gentle swirling. Cells were transferred to 50 ml conical tubes with 2 ml of FCS to stop the trypsin activity. Tubes were centrifuged at 1000 rpm for 5 minutes to pellet the cells. The supernatant was discarded.

Cells equal or less than 1 gram were lysed with 20 ml of TRIzol reagent (BRL), homogenized for 30 seconds, and extracted with 4 ml of chloroform. The tubes were centrifuged at 4000 rpm for 30 minutes and the aqueous phase was transferred to a new tube. RNA was precipitated by adding 10 ml isopropanol, mixing, and centrifuging for 30 minutes at 4200 rpm. The RNA pellet was washed with 10 ml of 70% ethanol, dried briefly, and resuspended in 0.5 ml TE buffer. Poly A⁺ RNA was prepared from 600 μg of total RNA by using a commercially available mRNA purification kit (Pharmacia). After elution of poly A⁺ RNA from the column in 750 μl of TE buffer, the sample was then ethanol precipitated in two 1.5 ml tubes by adding 40 μl sample buffer and 1 ml ethanol at −70° C. overnight.

The cDNA inserts of the positive clones were rescued by RT-PCR. A SuperScript™ preamplification system (BRL) was used to synthesize first strand cDNA. For each reaction, 1 μg polyA⁺ RNA, 1 μl (2 μM) vector specific primer 1605-21: 5′ AATCCGATGCCCACGTTGCAGTA 3′(SEQ ID NO: 2), and water were combined in a total volume of 15 μl. The mixture was incubated at 70° C. for 10 minutes and transferred to 50° C. The premixture containing 2.5 μl 10×buffer, 2.5 μl of 25 mM MgCl2, 1.3 μl 10 mM dNTPs, and 2.5 μl 0.1 M dithiotheritol was added. The reaction was started by addition of 1.2 μl reverse transcriptase and incubated at 50° C. for 1 hour. The reaction was stopped by incubation at 70° C. for 15 minutes. The RNA was digested with 1 μl Rnase H at 37° C. for 20 minutes.

PCR was performed with Pfu polymerase (Perkin Elmer). In a total volume of 100 μl 2 μl first strand reaction, 1×Pfu buffer, 0.5 μM each of primers 1239-08: 5′ AAAATCTTAGACCGACGACTGTGTTT 3′(SEQ ID NO: 3 ), and 1605-22 : 5′ GAGTCTCCGCAGCCTTTTGAGG (SEQ ID NO: 4), 0.2 mM dNTPs, 5% DMS, and 2.5 μl Pfu polymerase were added. The sample was heated at 95° C. for 1 minute, and amplified for 30 cycles. Each cycle includes: 95° C. for 30 seconds, 66° C. for 45 seconds, 72° C. for 2 minutes. The reaction was incubated at 70° C. for 10 minutes at the end.

PCR DNA fragments were extracted once with phenol/chloroform (50/50) and ethanol precipitated. The DNA was then digested with NotI and SalI and small fragments and PCR primers were removed by using mini-Q column on FPLC as described above. A signal trap library was constructed by ligating the DNA fragments into Sal I and Not I digested vector, ΔkFGF7L. Each ligation included 10 ng PCR fragments, 50 ng vector, 1×ligase buffer, and 0.5 μl T4 DNA ligase in a total volume of 10 μl. The ligation was carried at 16° C. overnight. The ligated DNA was precipitated by adding 5 μl tRNA, 10 μl water, 12.5 μl 7.5 M NH₄OAC, and 70 μl ethanol (−20° C.), and centrifuged for 20 minutes. The pellet was washed with 0.5 ml 70% ethanol (−20° C.), and resuspended in 5 μl water. One μl was used to transform 20 μl of E. coli DH10B cells by electroporation. More than 1 million cfu were obtained.

Plasmid DNA was prepared, and analyzed by DNA sequencing. The sequences were analyzed by computer to identify novel genes containing signal sequences and transmembrane domains. Clones having either signal sequences or transmembrane domains together comprised about 25% of the clones sequenced. It is estimated that about 1-5% of the total clones in the normal cDNA library contain signal sequence. A clone, ymkz5-00013-g11 (SEQ ID NO.:5), was isolated using the protocol described above and analyzed by DNA sequencing. The clone, ymkz5-00013-g11 was shown to contain an insert of 357 nucleotides (SEQ ID NO.: 5) that was predicted to encode 117 amino acids (SEQ ID NO.: 6).

A full length ymkz5 cDNA was subsequently isolated by the signal trap method from an E11 Mouse placental cDNA library using the 357 bp ymkz5-00013-g11 cDNA clone as a probe.

### EXAMPLE 2

### DNA Encoding Full Length Mouse ymkz5-Receptor

A cDNA library was prepared from mouse E11 placenta RNA by the method described in Example 1. The cDNA was fractionated using a 1% agarose gel, fragments were recovered using an NA45 ion exchange membrane (Schleicher & Schuell) according to the manufacturer's suggested protocol, and cDNA greater than 1.6 kb was ligated into vector pSPORT previously digested with SalI and NotI. The 357 bp ymkz5-00013-g11 fragment described in Example 1 was labeled with ³²P using a RadPrime kit (Gibco-BRL) according to the manufacturer's suggested protocol, and one million colonies were screened using standard protocols (Molecular Cloning, Maniatis et al.).

A cDNA clone containing the entire coding region of ymkz5 was isolated and is set out in SEQ ID NO: 7. The sequence includes an open reading frame of 531 nucleotides that encode a protein of 177 amino acids (SEQ ID NO: 8), in addition to 3 bp in the 5′ untranslated region and 431 bp in the 3′ untranslated region.

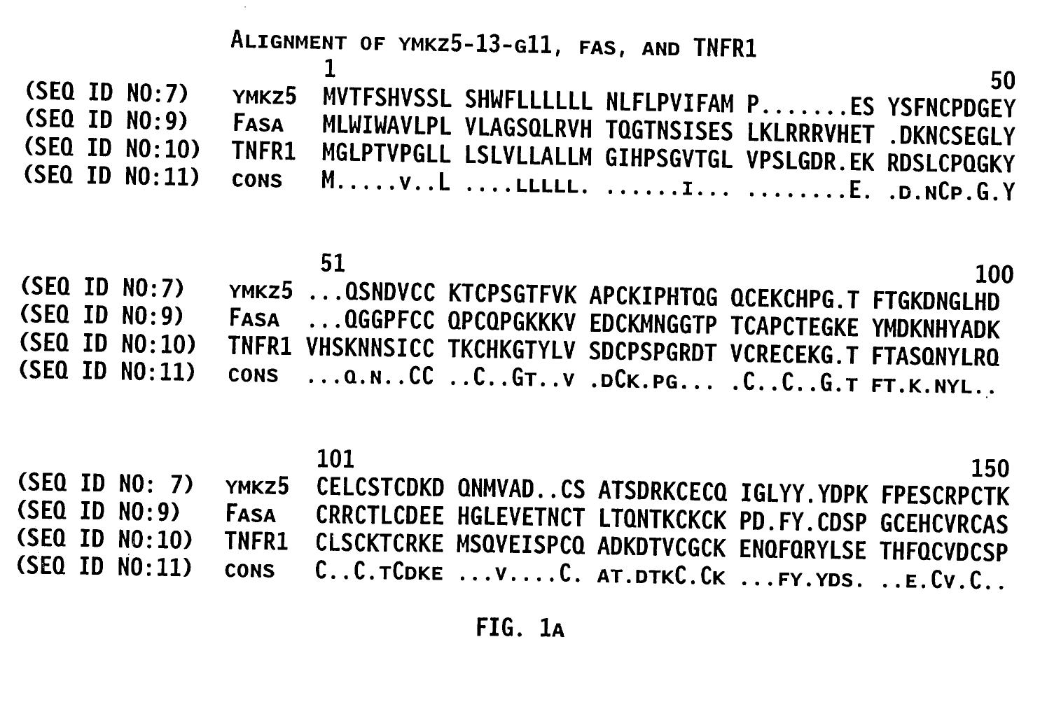

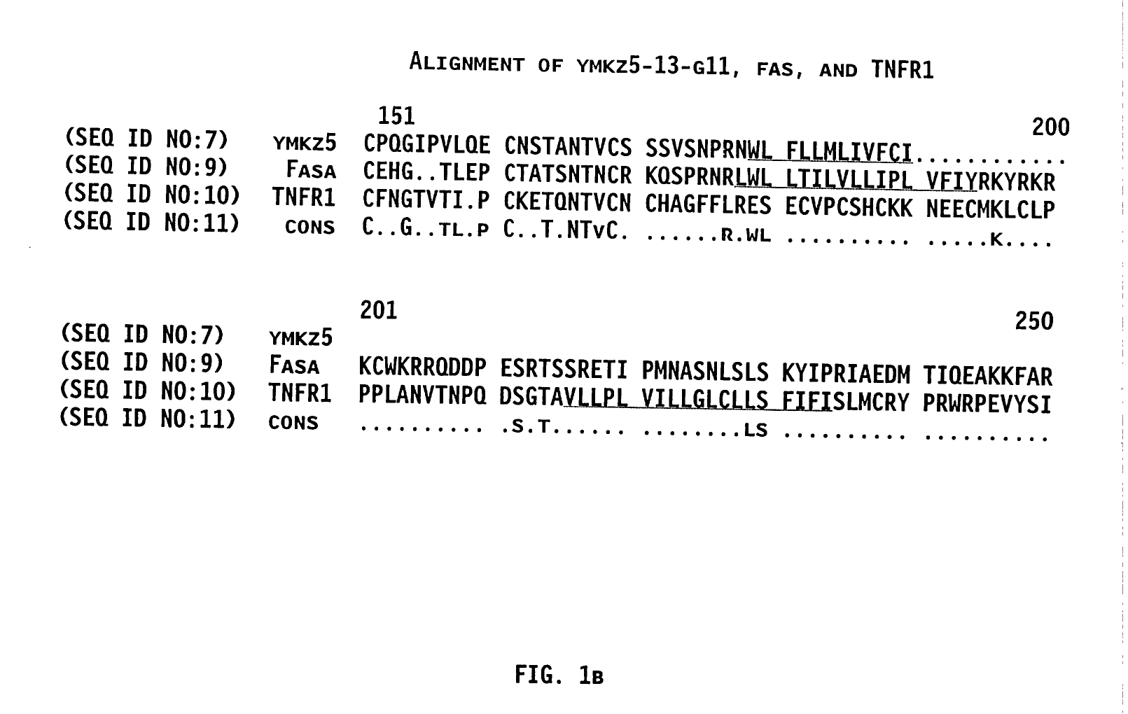

Computer analysis of the deduced ymkz5 amino acid sequence indicated that the protein is a novel member of the TNF receptor (TNFR) gene family. It is most closely related to Fas and the TNFR1. Alignment of the predicted amino acid sequence of ymkz5 with Fas and TNFR1 is shown in FIG. 1. The ymkz5 proteins includes an amino terminal signal peptide and a potential membrane anchor domain of 11 hydrophobic amino acid residues. The sequence of ymkz5 is approximately 27% identical with the extracellular domains of Fas and NFR1. All cysteine residues in Fas and TNFR1 are conserved in ymkz5. Unlike Fas and TNFR1, however, the structure of ymkz5 does not include an intracellular domain. The sequence of ymkz5 also shares a high degree of homology with another novel gene, tmst2 that was isolated from a mouse stromal cell line using the same method. Alignment of ymkz5 and tmst2 indicates that the two proteins share almost 80% identity at the amino acid level.

Comparison of the deduced amino acid structure of ymkz5 (SEQ ID NO: 8) with members of the TNF-receptor gene family reveals that it is most closely related to Fas (SEQ ID NO: 9) and TNFR1 (SEQ ID NO: 10). Comparison of the three sequences gives rise to a consensus sequence of conserved amino acid residues as set out in SEQ ID) NO: 11.

### EXAMPLE 3

### Tissue Specific Expression of ymkz5-Receptor

Tissue specific expression patterns of ymkz5-receptor gene was investigated by Northern blot analysis and in situ hybridization using a ³²P-labeled probe to detect the presence of ymkz5-receptor transcript in various tissues.

Cytoplasmic and poly-A⁺ RNA were isolated from placenta, developing embryos, and various adult tissues using standard techniques [Sambrook, J. et al, Molecular Cloning, Cold Spring Harbor Laboratory Press, New York (1989)]. Cells/tissues were lysed with 20 ml of TRlzol reagent (BRL), homogenized for 30 seconds, and extracted with 4 ml of chloroform. The tubes were centrifuged at 4000 rpm for 30 minutes and the aqueous phase was transferred to a new tube. RNA was precipitated by adding 10 ml isopropanol, mixing, and centrifuging for 30 minutes at 4200 rpm. The RNA pellet was washed with 10 ml of 70% ethanol, dried briefly, and resuspended in 0.5 ml TE buffer. Poly A⁺ RNA was prepared using a commercially available mRNA purification kit (Pharmacia). After elution of poly A⁺ RNA from the column in 750 μl of TE buffer, the sample was then ethanol precipitated by adding 40 μl sample buffer and 1 ml ethanol at −70° C. overnight. Poly A⁺ RNA was then fractionated using formaldehyde/agarose gel electrophoresis system as previously described and transferred. Following electrophoresis, the gel was processed and the RNA transferred to a nylon membrane. See Sambrook et al. Supra. Northern blots were then prehybridized in 20 ml of prehybridization solution containing 5×SSPE, 50% formamide, 5×Denhardt's solution, 0.5% SDS and 100 mg/ml denatured salmon sperm DNA for 2-4 hours at 42°0 C. The blots were then hybridized in 20 ml of hybridization solution containing 6×SSPE, 50% formamide, 5×Denhardt's solution, 0.5% SDS, 100 ug/ml denatured salmon sperm DNA. Approximately 5 ng/ml of random primed, ³²P-labeled (RadPrime Kit, GIBCO) ymkz5-00013-g11 cDNA was used as a probe. The blots were hybridized for 18-24 hours at 42° C. The blots were then washed in 0.1×SSC, 0.1% SDS at 55° C. The blots were then exposed to x-ray films for three days at 80° C.

Northern blot analysis revealed that ymkz5 gene is expressed at high levels in placenta and fetal kidney from 11-day embryos. In addition, moderate to low levels of ymkz5 transcripts were also detected in 7-day embryos, and in adult heart, lungs, small intestines and kidneys.

In situ hybridization analysis was also carried out to detect the presence and distribution of mRNA in different tissues. In situ hybridization was carried out as previously described. See Sambrook et al. Supra. Briefly, a panel of normal embryonic (E8.5 through E15.5) and adult mouse tissues were fixed in zinc-formalin fixative, embedded in paraffin, and sectioned to generate 5 μm thin sections. Following sectioning and prior to in situ hybridization, tissue sections were permeabilized with 0.2 M HCL, followed by digestion with Proteinase K. The sections were acetylated with triethanolamine and acetic anhydride. Sections were hybridized overnight at 55° C. with a ³²P-labeled riboprobe corresponding to the full length mouse cDNA that was generated using a Riboprobe kit (Promega) according to standard protocols. Excess probe was removed by RNase digestion followed by a series of washes in buffer with decreasing salt concentrations followed by a high stringent wash in 0.1×SSC at 55° C. The sections were then processed for autoradiography. The sections were dipped in Kodak NTB2 photographic emulsion, and kept a 4° C. for approximately 2-3 weeks. The sections were then developed and counterstained with hematoxilyn and eosin. Sections were examined using darkfield and transmitted light microscopy for tissue morphology and hybridization signals.

In situ hybridization showed high level of ymkz5 expression in trophoblasts and decidual cells of the placenta, with the signal observed at an 8.5 days of embryonic development stronger than at 14.5 days. The signal in all embryonic tissues was slightly above background. In adult tissues, moderately high levels of ymkz5 expression were detected in the ovary, uterus, and testis, while low levels of ymkz5 expression seen in the spleen, thymus, and lymph nodes.

### EXAMPLE 4

### Production of ymkz5-Receptor Polypeptides

A. Expression of ymkz5-Receptor Polypeptide in Bacteria

PCR may be used to amplify template DNA sequences encoding an ymkz5-receptor polypeptide using primers corresponding to the 5′ and 3′ ends of the sequence. The amplified DNA products may be modified to contain restriction enzyme sites to allow for insertion into expression vectors. PCR products are gel purified and inserted into expression vectors using standard recombinant DNA methodology. An exemplary vector, such as pAMG21I containing the lux promoter and a gene encoding kanamycin resistance is digested with BamHI and NdeI for directional cloning of inserted DNA. The ligated mixture is transformed into E. coli host strain 393 by electroporation and transformants selected for kanamycin resistance. Plasmid DNA from selected colonies is isolated and subjected to DNA sequencing to confirm the presence of the insert.

Transformed host cells are incubated in 2XYT medium containing 30 μg/ml kanamycin at 30° C. prior to induction. Gene expression can then be induced by addition of N-(3-oxohexanoyl)-d1-homoserine lactone to a final concentration of 30 ng/ml followed by incubation at either 30° C. or 37° C. for six hours. Expression of ymkz5-receptor polypeptide is evaluated by centrifugation of the culture, resuspension and lysis of the bacterial pellets, and analysis of host cell proteins by SDS-polyacrylamide gel electrophoresis.

Inclusion bodies containing ymkz5-receptor polypeptide are purified as follows: Bacterial cells are pelleted by centrifugation and resuspended in water. The cell suspension is lysed by sonication and pelleted by centrifugation at 195,000×g for 5 to 10 minutes. The supernatant is discarded and the pellet washed and transferred to a homogenizer. The pellet is homogenized in 5 ml. of a Percoll solution (75% liquid Percoll. 0.15 M NaCl) until uniformly suspended and then diluted and centrifuged at 21,600×g for 30 minutes. Gradient fractions containing the inclusion bodies are recovered and pooled. The isolated inclusion bodies are analyzed by SDS-PAGE.

B. Expression of ymkz5-Receptor Polypeptide in Mammalian Cells

A cDNA fragment encoding the first 161 amino acids of ymkz5 was prepared using PCR. Briefly, the ymkz5 cDNA region was PCR amplified using two primers having the SEQ IN NOS: 13 and 14, as set out below, and corresponding to the 5′ and 3′-ends of the cDNA sequence. Primer1 2026-70: ggtaagcttcaccatggttaccttcagccacgtc (SEQ ID NO: 12) Primer2 2026-69: gaattagcggccgcatttgaaacagatgaactgcacacac ( SEQ ID NO:13) The resulting fragment was digested with NotI and HindIII, and ligated into a pCept4 plasmid vector containing a DNA insert encoding the human Fc region. The resulting fusion gene was confirmed by DNA sequencing. The deduced amino acid sequence of the ymkz5-Fc fusion protein is set out in SEQ ID NO: 14. The construct was then transfected into 293-EBNA-1 cells (Invitrogen) by calcium phosphate method as previously described (Ausubel et al., Curr. Prot. Mol. Biol. 1, 9.11-9.13, (1994)). The transfected cells were then selected in 100 μg/ml hygromycin and the resulting drug-resistant cultures were grown to confluence. The cells were then washed in phosphate buffered saline (PBS) once and then cultured in serum-free media for 72 hours, the conditioned media removed, and ymkz5-Fc fusion protein was purified by protein-A column chromatography (Pierce) using the manufacturer's recommended procedures. Small amount of recombinant ymkz5-Fc fusion protein was produced by transient expression in 293-EBNA-1 cells.

The plasmid DNA was transfected into 293-EBNA-1 cells with Effectene reagent (QIAGEN) according to the manufacturer's suggested protocol. The cells were changed with 4 ml/ 100 mm plate serum-free medium. The conditioned medium was harvested after 2 days incubation. The recombinant protein in the conditioned medium was detected by Western blot using an anti-human Fc antibody. A single peptide about 50 kDa was detected by the Western blot.

If desired, ymkz5-receptor polypeptides may be excised from an SDS-polyacrylamide gel, or ymkz5-receptor fusion proteins may be purified by affinity chromatography and subjected to and subjected to amino acid sequence analysis as previously described..

### EXAMPLE 5

### Production of Anti-ymkz5-receptor Antibodies

Antibodies to ymkz5-receptor polypeptides may be obtained by immunization with purified protein or with ymkz5-receptor peptides produced by biological or chemical synthesis. Substantially pure ymkz5 protein or polypeptide may be isolated from transfected cells as described in Example 4. Concentration of protein in the final preparation may be adjusted, for example, by concentration on an Amicon filter device, to the level of a few micrograms/ml. Monoclonal or polyclonal antibodies to the protein can then be prepared by any of the procedures known in the art for generating antibodies, such as those described in Hudson and Bay, “Practical Immunology, Second Edition”, Blackwell Scientific Publications.

### A. Anti-ymkz5 Monoclonal Antibody Production

A monoclonal antibody to an epitope of any of the peptides identified and isolated as described can be prepared from murine hybridomas according to the classical method of Kohler, G. and Milstein, C., Nature 256:495 (1975) or derivative methods thereof. Briefly, a mouse is repetitively innoculated with a few micrograms of the selected protein over a period of a few weeks. The mouse is then sacrificed, and the antibody producing cells of the spleen isolated. The spleen cells are fused by means of polyethylene glycol with mouse myeloma cells such as NS-1 cells, and the excess unfused cells destroyed by growth of the system on selective media comprising hypoxanthine; aminopterin; thymidine (HAT media). The successfully fused cells are diluted and aliquots of the dilution placed in wells of a microtiter plate where growth of the culture is continued. After selection, tissue culture supernatants are taken from each fusion well and tested for ymkz5-receptor antibody production by EIA. Selected positive clones can be expanded and their monoclonal antibody product harvested for use. Detailed procedures for monoclonal antibody production are described in Davis, L. et al. Basic Methods in Molecular Biology, Section 21-2, Elsevier, New York. N.Y.

B. Polyclonal Anti-ymkz5 Receptor Antibody Production

Polyclonal antiserum containing antibodies to heterogenous epitopes of a single protein can be prepared by immunizing suitable animals with the expressed protein described above, which can be unmodified or modified to enhance immunogenicity. Effective polyclonal antibody production is affected by many factors related both to the antigen and the host species. For example, small molecules tend to be less immunogenic than large molecules and may require the use of carriers or adjuvants. Also, host animals vary in response to site of inoculations and dose, with both inadequate or excessive doses of antigen resulting in low titer antisera. Small doses (ng levels) of antigen administered at multiple intradermal sites appear to be most reliable. An effective immunization protocol for rabbits can be found in Vaitukaitis, J. et al. J Clin. Endocrinol. Metab. 33: 988-991 (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology ed. D. Weir, Blackwell (1973). Plateau concentration of antibody is usually in the range of 0.1 to 0.2 mg/ml of serum (about 12 um). Affinity of the antisera for the antigen is determined by preparing competitive binding curves, as described, for example, by Fisher, D., Chapt. 42 in; Manual of Clinical Immunology, 2d Ed. (Rose and Friedman, eds.) Amer. Soc. For Microbiol., Washington, D.C. (1980).

Alternative procedures for obtaining anti-ymkz5-receptor antibodies may also be employed, such as immunization of transgenic mice harboring human Ig loci for production of fully human antibodies, and screening of synthetic antibody libraries, such as those generated by mutagenesis of an antibody variable domain.

### EXAMPLE 6

### Biological Activity of ymkz5-Receptor Polypeptide

Full length ymkz5-Fc receptor fusion was expressed as described above in 293 cells and tested in an in vitro binding assay for the ability to bind members of the TNF related ligand family. In preliminary studies, the ymkz5-receptor was shown to bind TNF-related apoptosis inducing ligand (TRAIL). The binding assay was carried out as follows.

Conditioned medium containing the recombinant ymkz5-Fc protein was concentrated 10 times with a Contriplus concentrator (AMICON). The 10x conditioned medium was incubated with COS-7 cells transfected with mTRAIL/pCept4 plasmid DNA the day before for 1 hour at 4° C. The cells were washed 3 times with PBS, and incubated with an alkaline phosphatase (AP) conjugated anti-human Fc antibody (PIECE) for 30 minutes. The cells were then washed with 1×TBS 3 times. The binding of the fusion protein was visualized with adding Fast Red reagent (PIERCE).

The results showed that recombinant Fc fusion protein was bound only to cells transfected to TRAIL expressed on COS-7 cell. The binding was specific since the protein did not bind to the cells transfected with 3 other members of the TNF family. This observation suggest that the ymkz5-receptor fusion protein produced has biological activity, and may be involved in regulating the effects of TNF-related ligand, TRAIL.

### EXAMPLE 7

### Functional Analysis of the Role of ymkz5-Receptor

To determine the functional role of ymkz5 in vivo, the ymkz5 gene is either over expressed in the germ line of animals or inactivated in the germ line of mammals by homologous recombination. Animals in which the gene is over expressed, under the regulatory control of exogenous or endogenous promoter elements, are known as transgenic animals. Animals in which an endogenous gene has been inactivated by homologous recombination are also known as “knockout” animals. Exemplary mammals include rabbits and rodent species such as mice.

Transgenic animals allow for the determination of the effect(s) of over expression or inappropriate expression of the ymkz5-receptor on development and disease processes. ymkz5-receptor transgenic animals can also serve as a model system to test compounds that can modulate receptor activity.

The “knockout” animals allow for the determination of the role of ymkz5 in embryonic development, and in immune and proliferative responses. The role of ymkz5 in development, and in immune and proliferative responses, is determined by analysis the effect(s) of gene knockout on the development of the embryo as well as on the development and differentiation of various organs and tissues such as the immune system in these animals (as determined by FACS analysis of cell populations at different stages of development).

### EXAMPLE 8

### Binding Analysis of TNF Ligand Family Members with ymkz5-Receptor

Binding studies were performed to determine if various TNF ligand family members are ligands for the ymkz5-receptor. The only TNF ligand which bound to the ymkz5-receptor was murine TRAIL. The binding studies, based on surface plasmon resonance, were carried out with the automated, high throughput Biacore 2000 system at 25° C. according to the manufacturer's instructions (Biacore, Uppsula, Sweden) as follows:

The receptor was immobilized on a CM5 research grade amine coupling chip (Biacore) by placing 40 μg/ml of recombinant ymkz5-receptor diluted in HEPES buffered saline (HBS-P; Biacore) at pH 4.5 on the chip. The ligands were diluted in HSP-B buffer containing 10 μg/ml BSA and 4 mg/ml dextran to block nonspecific binding sites. The ligand samples were injected over the receptors at concentrations ranging from 2 nM to 100 nM. The chips were regenerated between ligand injections by washing 2 times for 3 minutes in 25 mM CAPS, 1 M NaCl pH 10.5.

The TNF ligands tested were: human flag TRAIL (amino acids 95-281, Genbank accession no. AAC50332), murine flag TRAIL (amino acids 99-291, Genbank accession no. NP 033451), murine Fc TRAIL, human OPGL ligand (amino acids 159-318), murine OPGL ligand (amino acids 159-316), human TNFα (amino acids 82-233, Genbank accession no. CAA26669), and murine TNFα (amino acids 82-233,, Genbank accession no. CAA68530). The extracellular and transmembrane portions of the ligands were recombinantly expressed in E.coli. Specifically, the denoted amino acids for each ligand indicate the portion of the ligand expressed recombinantly. Additionally, commercially available human Fas ligand (Alexis Biochemicals, San Diego, Calif.) was also tested.. Human DR5Fc, a known TRAIL receptor, was used as a control. Results of the assay were determined by detecting the change in mass on the chip as measured by changes in light absorption on the chip as indicated as resonance units. The ymkz5-receptor only bound to active murine TRAIL which was bioactive in cell culture assays, and the KD was 20.8 nM. The species specific binding suggests that ymkz5 may function as a TRAIL decoy receptor.

Similar species specific binding to TRAIL was demonstrated for tmst2, a novel transmembrane TNF receptor, cloned by Amgen, which is closely linked to ymkz5 in the murine genome. Primary sequences homology comparisons indicate that both ymkz5 and tmst2 are most closely related to FAS and TNFR-1 which are not functionally similar. Therefore, the characterization of the murine genes, ymkz5 and tmst2, may aid in the discovery of human TRAIL decoy receptors based on functionality and not solely based on primary sequence homology.

While the present invention has been described in terms of the preferred embodiments, it is understood that variations and modifications will occur to those skilled in the art. Therefore, it is intended that the appended claims cover all such equivalent variations which come within the scope of the invention as claimed.

What is claimed:

1. An isolated nucleic acid molecule comprising a nucleotide sequence selected from the group consisting of:
(a) the nucleotide sequence as set forth in SEQ ID NO: 7;
(b) a nucleotide sequence encoding the polypeptide as set forth in SEQ ID NO: 8;
(c) a nucleotide sequence which hybridizes under moderately or highly stringent conditions to the complement of (a) or (b), wherein the encoded polypeptide has an activity of the polypeptide as set forth in SEQ ID NO: 8; and
(d) a nucleotide sequence complementary to any of (a)-(c).

2. An isolated nucleic acid molecule comprising a nucleotide sequence selected from the group consisting of:
(a) a nucleotide sequence encoding a polypeptide that is at least about 70, percent identical to the polypeptide as set forth in SEQ ID NO: 8, wherein the polypeptide has an activity of the polypeptide as set forth in SEQ ID NO: 8;
(b) a nucleotide sequence encoding an allelic variant or splice variant of the nucleotide sequence as set forth in SEQ ID NO: 7, wherein the encoded polypeptide has an activity of the polypeptide as set forth in SEQ ID NO: 8;
(c) a nucleotide sequence of SEQ ID NO: 7; (a); or (b) encoding a polypeptide fragment of at least about 25 amino acid residues, wherein the polypeptide has an activity of the polypeptide as set forth in SEQ ID NO: 8;
(d) a nucleotide sequence of SEQ ID NO: 7, or (a)-(c) comprising a fragment of at least about 16 nucleotides;
(e) a nucleotide sequence which hybridizes under moderately or highly stringent conditions to the complement of any of (a)-(d), wherein the polypeptide has an activity of the polypeptide as set forth in SEQ ID NO: 8; and
(f) a nucleotide sequence complementary to any of (a)-(c).

3. An isolated nucleic acid molecule comprising a nucleotide sequence selected from the group consisting of:
(a) a nucleotide sequence encoding a polypeptide as set forth in SEQ ID NO: 8 with at least one conservative amino acid substitution, wherein the polypeptide has an activity of the polypeptide as set forth in SEQ ID NO: 8;
(b) a nucleotide sequence encoding a polypeptide as set forth in SEQ ID NO: 8 with at least one amino acid insertion, wherein the polypeptide has an activity of the polypeptide as set forth in SEQ ID NO: 8;
(c) a nucleotide sequence encoding a polypeptide as set forth in SEQ ID NO: 8 with at least one amino acid deletion, wherein the polypeptide has an activity of the polypeptide as set forth in SEQ ID NO: 8;
(d) a nucleotide sequence encoding a polypeptide as set forth in SEQ ID NO: 8 which has a C- and/or N-terminal truncation, wherein the polypeptide has an activity of the polypeptide as set forth in SEQ ID NO: 8;
(e) a nucleotide sequence encoding a polypeptide as set forth in SEQ ID NO: 8 with at least one modification selected from the group consisting of amino acid substitutions, amino acid insertions, amino acid deletions, C-terminal truncation, and N-terminal truncation, wherein the polypeptide has an activity of the polypeptide as set forth in SEQ ID NO: 8;
(f) a nucleotide sequence of (a)-(e) comprising a fragment of at least about 16 nucleotides;
(g) a nucleotide sequence which hybridizes under moderately or highly stringent conditions to the complement of any of (a)-(f), wherein the polypeptide has an activity of the polypeptide as set forth in SEQ ID NO: 8; and
(h) a nucleotide sequence complementary to any of (a)-(e).

4. A vector comprising the nucleic acid molecule of claims 1, 2, or 3.

5. A host cell comprising the vector of claim 4.

6. The host cell of claim 5 that is a eukaryotic cell.

7. The host cell of claim 5 that is a prokaryotic cell.

8. A process of producing a ymkz5-receptor polypeptide comprising culturing the host cell of claim 5 under suitable conditions to express the polypeptide, and optionally isolating the polypeptide from the culture.

9. A polypeptide produced by the process of claim 8.

10. The process of claim 8, wherein the nucleic acid molecule comprises promoter DNA other than the promoter DNA for the native ymkz5-receptor polypeptide operatively linked to the DNA encoding the ymkz5-receptor polypeptide.

11. The isolated nucleic acid molecule according to claim 2 wherein the percent identity is determined using a computer program selected from the group consisting of GAP, BLASTP, BLASTN, FASTA, BLASTA, BLASTX, BestFit, and the Smith-Waterman algorithm.

12. A process for identifying candidate inhibitors of ymkz5-receptor polypeptide activity or production comprising exposing a cell according to claims 5, 6, or 7 to the candidate inhibitors, and measuring ymkz5-receptor polypeptide activity or production in said cell, comparing activity or production in the presence and absence of the candidate.

13. An isolated polypeptide comprising the amino acid sequence set forth in SEQ ID NO: 8.

14. An isolated polypeptide comprising the amino acid sequence selected from the group consisting of:
(a) the mature amino acid sequence as set forth in SEQ ID NO: 8. comprising a mature amino terminus at residue 1, optionally further comprising an amino-terminal methionine;
(b) an amino acid sequence for an ortholog of SEQ ID NO: 8, wherein the encoded polypeptide has an activity of the polypeptide as set forth in SEQ ID NO: 8;
(c) an amino acid sequence that is at least about 70 percent identical to the amino acid sequence of SEQ ID NO: 8, wherein the polypeptide has an activity of the polypeptide as set forth in SEQ ID NO: 8;
(d) a fragment of the amino acid sequence set forth in SEQ ID NO: 8 comprising at least about 25 amino acid residues, wherein the polypeptide has an activity of the polypeptide as set forth in SEQ ID NO: 8;
(e) an amino acid sequence for an allelic variant or splice variant of either the amino acid sequence as set forth in SEQ ID NO: 8, or at least one of (a)-(c) wherein the polypeptide has an activity of the polypeptide as set forth in SEQ ID NO: 8.

15. An isolated polypeptide comprising the amino acid sequence selected from the group consisting of:
(a) the amino acid sequence as set forth in SEQ ID NO: 8 with at least one conservative amino acid substitution, wherein the polypeptide has an activity of the polypeptide as set forth in SEQ ID NO: 8;
(b) the amino acid sequence as set forth in SEQ ID NO: 8 with at least one amino acid insertion, wherein the polypeptide has an activity of the polypeptide as set forth in SEQ ID NO: 8;
(c) the amino acid sequence as set forth in SEQ ID NO: 8 with at least one amino acid deletion, wherein the polypeptide has an activity of the polypeptide as set forth in SEQ ID NO: 8;
(d) the amino acid sequence as set forth in SEQ ID NO: 8 which has a C- and/or N-terminal truncation, wherein the polypeptide has an activity of the polypeptide as set forth in SEQ ID NO: 8; and
(e) the amino acid sequence as set forth in SEQ ID NO: 8, with at least one modification selected from the group consisting of amino acid substitutions, amino acid insertions, amino acid deletions, C-terminal truncation, and N-terminal truncation wherein the polypeptide has an activity of the polypeptide as set forth in SEQ ID NO: 8.

16. An isolated polypeptide encoded by the nucleic acid molecule of claims 1, 2, or 3.

17. The isolated polypeptide according to claim 14 wherein the percent identity is determined using a computer program selected from the group consisting of GAP, BLASTP, BLASTN, FASTA, BLASTA, BLASTX, BestFit, and the Smith-Waterman algorithm.

18. An antibody produced by immunizing an animal with a peptide comprising an amino acid sequence of SEQ ID NO: 8.

19. An antibody or fragment thereof that specifically binds the polypeptide of claims 13, 14, or 15.

20. The antibody of claim 19 that is a monoclonal antibody.

21. A hybridoma that produces a monoclonal antibody that binds to a peptide comprising an amino acid sequence of SEQ ID NO: 8.

22. A method of detecting or quantitating the amount of ymkz5-receptor polypeptide using the anti-ymkz5-receptor antibody or fragment of claims 18, 19, or 20.

23. A selective binding agent or fragment thereof that specifically binds at least one polypeptide wherein said polypeptide comprises the amino acid sequence selected from the group consisting of:
(a) the amino acid sequence as set forth in SEQ ID NO: 8;
(b) a fragment of the amino acid sequence set forth in at least one of SEQ ID NO: 8; and
(c) a naturally occurring variant of (a) or (b).

24. The selective binding agent of claim 23 that is an antibody or fragment thereof.

25. The selective binding agent of claim 23 that is a humanized antibody.

26. The selective binding agent of claim 23 that is a human antibody or fragment thereof.

27. The selective binding agent of claim 23 that is a polyclonal antibody or fragment thereof.

28. The selective binding agent claim 23 that is a monoclonal antibody or fragment thereof.

29. The selective binding agent of claim 23 that is a chimeric antibody or fragment thereof.

30. The selective binding agent of claim 23 that is a CDR-grafted antibody or fragment thereof.

31. The selective binding agent of claim 23 that is an antiidiotypic antibody or fragment thereof.

32. The selective binding agent of claim 23 which is a variable region fragment.

33. The variable region fragment of claim 32 which is a Fab or a Fab′ fragment.

34. A selective binding agent or fragment thereof comprising at least one complementarity determining region with specificity for a polypeptide having the amino acid sequence of SEQ ID NO: 8.

35. The selective binding agent of claim 23 which is bound to a detectable label.

36. The selective binding agent of claim 23 which antagonizes ymkz5-receptor polypeptide biological activity.

37. A method for treating, preventing, or ameliorating a disease, condition, or disorder comprising administering to a patient an effective amount of a selective binding agent according to claim 23.

38. A selective binding agent produced by immunizing an animal with a polypeptide comprising an amino acid sequence of SEQ ID NO: 8.

39. A hybridoma that produces a selective binding agent capable of binding a polypeptide according to claims 1, 2, or 3.

40. A composition comprising the polypeptide of claims 13, 14, or 15 and a pharmaceutically acceptable formulation agent.

41. The composition of claim 40 wherein the pharmaceutically acceptable formulation agent is a carrier, adjuvant, solubilizer, stabilizer, or anti-oxidant.

42. The composition of claim 40 wherein the polypeptide comprises the mature amino acid sequence as set forth in SEQ ID NO: 8.

43. A polypeptide comprising a derivative of the polypeptide of claims 13, 14, or 15.

44. The polypeptide of claim 43 which is covalently modified with a water-soluble polymer.

45. The polypeptide of claim 44 wherein the water-soluble polymer is selected from the group consisting of polyethylene glycol, monomethoxy-polyethylene glycol, dextran, cellulose, poly-(N-vinyl pyrrolidone) polyethylene glycol, propylene glycol homopolymers, polypropylene oxide/ethylene oxide co-polymers, polyoxyethylated polyols, and polyvinyl alcohol.

46. A composition comprising a nucleic acid molecule of claims 1, 2, or 3 and a pharmaceutically acceptable formulation agent.

47. A composition of claim 46 wherein said nucleic acid molecule is contained in a viral vector.

48. A viral vector comprising a nucleic acid molecule of claims 1, 2, or 3.

49. A fusion polypeptide comprising the polypeptide of claims 13, 14, or 15 fused to a heterologous amino acid sequence.

50. The fusion polypeptide of claim 49 wherein the heterologous amino acid sequence is an IgG constant domain or fragment thereof.

51. A method for treating, preventing or ameliorating a medical condition in a mammal resulting from decreased levels of ymkz5-receptor polypeptide comprising administering to a patient the polypeptide of claims 13, 14, or 15 or the polypeptide encoded by the nucleic acid of claims 1, 2, or 3 to said mammal.

52. A method of diagnosing a pathological condition or a susceptibility to a pathological condition in a subject caused by or resulting from abnormal levels of ymkz5-receptor polypeptide comprising:
(a) determining the presence or amount of expression of the polypeptide of claims 13, 14, or 15 or the polypeptide encoded by the nucleic acid molecule of claims 1, 2, or 3 in a sample; and
(b) comparing the level of ymkz5-receptor polypeptide in a biological, tissue or cellular sample from normal subjects or the subject at an earlier time, wherein susceptibility to a pathological condition is based on the presence or amount of expression of the polypeptide.

53. A device, comprising:
(a) a membrane suitable for implantation; and
(b) cells encapsulated within said membrane, wherein said cells secrete a protein of claims 13, 14, or 15, and wherein said membrane is permeable to said protein and impermeable to materials detrimental to said cells.

54. A device, comprising:
(a) a membrane suitable for implantation; and
(b) the ymkz5-receptor polypeptide encapsulated within said membrane, wherein said membrane is permeable to the polypepetide.

55. A method of identifying a compound which binds to a polypeptide comprising:
(a) contacting the polypeptide of claims 13, 14, or 15 with a compound; and
(b) determining the extent of binding of the polypeptide to the compound.

56. A method of modulating levels of a polypeptide in an animal comprising administering to the animal the nucleic acid molecule of claims 1, 2, or 3.

57. A transgenic non-human mammal comprising the nucleic acid molecule of claims 1, 2, or 3.

58. A diagnostic reagent comprising a detectably labeled polynucleotide encoding the amino acid sequence set out in SEQ ID NO: 8; or a fragment, variant or homolog thereof including allelic variants and spliced variants thereof.

59. The diagnostic reagent of claim 58, wherein said labeled polynucleotide is a first-strand cDNA.

60. A method for determine the presence of ymkz5-receptor nucleic acids in a biological sample comprising the steps of:
(a) providing a biological sample suspected of containing ymkz5-receptornucleic acids;
(b) contacting the biological sample with a diagnostic reagent according to claim 59 under conditions wherein the diagnostic reagent will hybridize with ymkz5-receptornucleic acids contained in said biological sample;
(c) detecting hybridization between ymkz5-receptornucleic acid in the biological sample and the diagnostic reagent; and
(d) comparing the level of hybridization between the biological sample and diagnostic reagent with the level of hybridization between a known concentration of ymkz5-receptor nucleic acid and the diagnostic reagent.

61. A method for detecting the presence of ymkz5-receptor nucleic acids in a tissue or cellular sample comprising the steps of:
(a) providing a tissue or cellular sample suspected of containing ymkz5-receptor nucleic acids;
(b) contacting the tissue or cellular sample with a diagnostic reagent according to claim 59 under conditions wherein the diagnostic reagent will hybridize with ymkz5-receptor nucleic acids;
(c) detecting hybridization between ymkz5-receptor nucleic acid in the tissue or cellular sample and the diagnostic reagent; and
(d) comparing the level of hybridization between the tissue or cellular sample and diagnostic reagent with the level of hybridization between a known concentration of ymkz5-receptor nucleic acid and the diagnostic reagent.

62. The method of claim 59 wherein said polynucleotide molecule is DNA.

63. The method of claim 59 wherein said polynucleotide molecule is RNA.
